(19) United States
(12) Patent Application Publication
(10) Pub. No.: US 2008/0255202 A1
(43) Pub. Date: Oct. 16, 2008

(54) Phenylthioacetic Acid Derivatives and Use Thereof

(75) Inventors: Hilmar Bischoff, Wuppertal (DE); Elke Dittrich-Wengenroth, Wuppertal (DE); Nils Griebenow, Dormagen (DE); Axel Kretschmer, Wuppertal (DE); Joachim Kruger, Dusseldorf (DE); Elisabeth Woltering, Hilden (DE); Lars Barfacker, Oberhausen (DE); Claudia Hirth-Dietrich, Wuppertal (DE); Peter Ellinghaus, Wuppertal (DE); Martin Raabe, Ulm (DE); Stephen Bartel, Kurten (DE); Christian Pilger, Ludwigshafen (DE); Ulrich Rosentreter, Wuppertal (DE); Klemens Lustig, Wuppertal (DE); Armin Kern, Wuppertal (DE); Dieter Lang, Velbert (DE)

Correspondence Address: Bayer Health Care LLC, 400 Morgan Lane, West Haven, CT, 06516, US

(73) Assignee: Bayer HealthCare AG, Leverkusen (DE)

(21) Appl. No.: 11/547,974

(22) PCT Filed: Mar. 26, 2005
(86) PCT No.: PCT/EP05/03226

§ 371 Date: Oct. 3, 2007

(30) Foreign Application Priority Data
Apr. 7, 2004 (DE) 102004016845.8

(51) Int. Cl. A61K 31/4427; C07D 271/06; A61K 31/4245; C07D 261/06; A61K 31/42; C07D 271/10; C07D 231/10; C07D 213/02; A61P 9/00; A61K 31/4155; C07D 277/20; A61K 31/427; C07D 249/08; A61K 31/4196; C07D 233/54

(52) U.S. Cl. 514/340; 548/131; 514/364; 548/247; 514/378; 548/143; 548/365.7; 514/406; 548/204; 514/365; 548/266.2; 514/383; 548/315.4; 514/397; 546/269.4

(57) ABSTRACT

The present application relates to novel phenylthioacetic acid derivatives, to processes for their preparation, to their use for the treatment and/or prophylaxis of diseases and to their use for preparing medicaments for the treatment and/or prophylaxis of diseases, preferably for the treatment and/or prevention of cardiovascular disorders, in particular dyslipidaemias and arteriosclerosis.

The present invention relates to novel phenylthioacetic acid derivatives, to processes for their preparation, to their use for the treatment and/or prophylaxis of diseases and to their use for preparing medicaments for the treatment and/or prophylaxis of diseases, preferably for the treatment and/or prevention of cardiovascular diseases, in particular dyslipidaemias and arteriosclerosis.

In spite of many successful therapies, cardiovascular disorders remain a serious public health problem. Treatment with statins, which inhibit HMG-CoA reductase, very successfully lowers both LDL cholesterol (LDL-C) plasma concentrations and the mortality of patients at risk; however, convincing treatment strategies for the therapy of patients having an unfavourable HDL-C/LDL-C ratio and/or hypertriglyceridaemia are still not available to date.

Currently, in addition to niacin, fibrates are the only therapy option for patients of these risk groups. They lower elevated triglyceride levels by 20-50%, reduce LDL-C by 10-15%, change the LDL particle size of atherogenic LDL of low density to less atherogenic LDL of normal density and increase the HDL concentration by 10-15%.

Fibrates act as weak agonists of the peroxysome-proliferator-activated receptor (PPAR)-alpha (Nature 1990, 347, 645-50). PPAR-alpha is a nuclear receptor which regulates the expression of target genes by binding to DNA sequences in the promoter range of these genes [also referred to as PPAR response elements (PPRE)]. PPREs have been identified in a number of genes coding for proteins which regulate lipid metabolism. PPAR-alpha is highly expressed in the liver, and its activation leads inter alia to lower VLDL production/secretion and reduced apolipoprotein CIII (ApoCIII) synthesis. In contrast, the synthesis of apolipoprotein A1 (ApoA1) is increased.

A disadvantage of fibrates which have hitherto been approved is that their interaction with the receptor is only weak (EC₅₀ in the μM range), which in turn is responsible for the relatively small pharmacological effects described above.

It was an object of the present invention to provide novel compounds suitable for use as PPAR-alpha modulators for the treatment and/or prevention of in particular cardiovascular disorders.

PPAR modulators having a thiazole partial structure are described in WO 01/40207, WO 02/46176, WO 02/096894, WO 02/096895, WO 03/072100, WO 03/072102, WO 2004/000785 and WO 2004/020420.

The present invention provides compounds of the general formula (I)

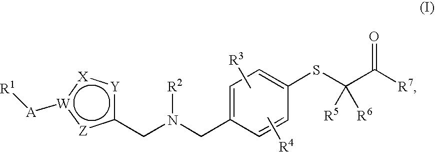

in which
W, X, Y and Z together with the carbon atom to which Y and Z are attached form a 5-membered heteroaryl ring which may optionally be mono- or disubstituted by identical or different substituents from the group consisting of (C₁-C₆)-alkyl and trifluoromethyl and in which
   W represents C or N
   and
   X, Y and Z each represent C, N, O or S,
   where at least one of the ring members W, X, Y and Z represents a heteroatom from the group consisting of N, O and S,
A, in the case that W represents C, represents a bond or represents CH₂, C(CH₃)₂, C(═O), O, S or NR⁸, in which
   R⁸ represents hydrogen or (C₁-C₆)-alkyl,
   and,
   in the case that W represents N, represents a bond or represents CH₂ or C(═O),
R¹ represents (C₆-C₁₀)-aryl or 5- to 10-membered heteroaryl which may each be substituted up to four times by identical or different substituents selected from the group consisting of halogen, nitro, cyano, (C₁-C₆)-alkyl, (C₃-C₈)-cycloalkyl, phenyl, pyridyl, hydroxyl, (C₁-C₆)-alkoxy, trifluoromethyl, trifluoromethoxy, amino, mono- and di-(C₁-C₆)-alkylamino, R⁹—C(O)—NH—, R¹⁰—C(O)—, R¹¹R¹²N—C(O)—NH— and R¹³R¹⁴N—C(O)—, in which
   R⁹ represents hydrogen, (C₁-C₆)-alkyl, (C₃-C₈)-cycloalkyl, phenyl or (C₁-C₆)-alkoxy,
   R¹⁰ represents hydrogen, (C₁-C₆)-alkyl, (C₃-C₈)-cycloalkyl, phenyl, hydroxyl or (C₁-C₆)-alkoxy
   and
   R¹¹, R¹², R¹³ and R¹⁴ are identical or different and independently of one another represent hydrogen, (C₁-C₆)-alkyl, (C₃-C₈)-cycloalkyl or phenyl,
R² represents hydrogen, (C₆-C₁₀)-aryl, (C₁-C₆)-alkyl, (C₂-C₆)-alkenyl or (C₂-C₆)-alkynyl, in which alkyl, alkenyl and alkynyl may each be substituted by trifluoromethyl, fluorine, cyano, (C₁-C₆)-alkoxy, trifluoromethoxy, (C₆-C₁₀)-aryl or 5- or 6-membered heteroaryl, where all aryl and heteroaryl groups mentioned for their part may in each case be substituted up to three times by identical or different substituents selected from the group consisting of halogen, nitro, cyano, (C₁-C₆)-alkyl, hydroxyl, (C₁-C₆)-alkoxy, trifluoromethyl and trifluoromethoxy,
R³ and R⁴ are identical or different and independently of one another represent hydrogen, (C₁-C₆)-alkyl, (C₂-C₆)-alkenyl, (C₁-C₆)-alkoxy, trifluoromethyl, trifluoromethoxy or halogen,
R⁵ and R⁶ are identical or different and independently of one another represent hydrogen, (C₁-C₆)-alkyl, (C₁-C₆)-alkoxy or phenoxy or together with the carbon atom to which they are attached form a (C₃-C₈)-cycloalkyl ring,
and
R⁷ represents a group of the formula —NHR¹⁵ or —OR¹⁶, in which
   R¹⁵ represents hydrogen, (C₁-C₆)-alkyl or (C₁-C₆)-alkylsulphonyl
   and
   R¹⁶ represents hydrogen or represents a hydrolyzable group which can be converted into the corresponding carboxylic acid,
      and their salts, solvates and solvates of the salts.

In the context of the invention, in the definition of R¹⁶, a hydrolyzable group denotes a group where, in particular in the body, the —C(O)OR¹⁶ grouping is converted into the corresponding carboxylic acid (R¹⁶=hydrogen). By way of example and by way of preference, such groups are benzyl, (C₁-C₆)-alkyl or (C₃-C₈)-cycloalkyl which are in each case optionally mono- or polysubstituted by identical or different substituents from the group consisting of halogen, hydroxyl, amino, (C₁-C₆)-alkoxy, carboxyl, (C₁-C₆)-alkoxycarbonyl, (C₁-C₆)-alkoxycarbonylamino and (C₁-C₆)-alkanoyloxy or, in particular, (C₁-C₄)-alkyl which is optionally mono- or polysubstituted by identical or different substituents from the group consisting of halogen, hydroxyl, amino, (C₁-C₄)-alkoxy, carboxyl, (C₁-C₄)-alkoxycarbonyl, (C₁-C₄)-alkoxycarbonylamino and (C₁-C₄)-alkanoyloxy.

Compounds according to the invention are the compounds of the formula (I) and their salts, solvates and solvates of the salts, the compounds, comprised by formula (I), of the formulae mentioned below and their salts, solvates and solvates of the salts and the compounds comprised by the formula (I), mentioned below as embodiments and their salts, solvates and solvates of the salts if the compounds, comprised by the formula (I), mentioned below are not already salts, solvates and solvates of the salts.

Depending on their structure, the compounds according to the invention can exist in stereoisomeric forms (enantiomers, diastereomers). Accordingly, the invention comprises the enantiomers or diastereomers and their respective mixtures. From such mixtures of enantiomers and/or diastereomers, it is possible to isolate the stereoisomerically uniform constituents in a known manner.

If the compounds according to the invention can be present in tautomeric forms, the present invention comprises all tautomeric forms.

In the context of the present invention, preferred salts are physiologically acceptable salts of the compounds according to the invention. The invention also comprises salts which for their part are not suitable for pharmaceutical applications, but which can be used, for example, for isolating or purifying the compounds according to the invention.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalene disulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of customary bases, such as, by way of example and by way of preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

In the context of the invention, solvates are those forms of the compounds according to the invention which, in solid or liquid state, form a complex by coordination with solvent molecules. Hydrates are a specific form of the solvates where the coordination is with water. In the context of the present invention, preferred solvates are hydrates.

Moreover, the present invention also comprises prodrugs of the compounds according to the invention. The term “prodrugs” includes compounds which for their part may be biologically active or inactive but which, during the time they spend in the body, are converted into compounds according to the invention (for example metabolically or hydrolytically).

In the context of the present invention, unless specified otherwise, the substituents have the following meanings:

In the context of the invention, (C₁-C₆)-alkyl and (C₁₋₄)-alkyl represent a straight-chain or branched alkyl radical having 1 to 6 and 1 to 4 carbon atoms, respectively. Preference is given to a straight-chain or branched alkyl radical having 1 to 4 carbon atoms. The following radicals may be mentioned by way of example and by way of preference: methyl, ethyl, n-propyl, isopropyl, n-butyl, iso-butyl, sec-butyl, tert-butyl, 1-ethylpropyl, n-pentyl and n-hexyl.

In the context of the invention, (C₂-C₆)-alkenyl and (C₂-C₄)-alkenyl represent a straight-chain or branched alkenyl radical having 2 to 6 and 2 to 4 carbon atoms, respectively. Preference is given to a straight-chain or branched alkenyl radical having 2 to 4 carbon atoms. The following radicals may be mentioned by way of example and by way of preference: vinyl, allyl, isopropenyl, n-but-2-en-1-yl and 2-methyl-2-propen-1-yl.

In the context of the invention, (C₂-C₆)-alkynyl and (C₁-C₄)-alkynyl represent a straight-chain or branched alkynyl radical having 2 to 6 and 2 to 4 carbon atoms, respectively. Preference is given to a straight-chain or branched alkynyl radical having 2 to 4 carbon atoms. The following radicals may be mentioned by way of example and by way of preference: ethynyl, n-prop-2-yn-1-yl, n-but-2-yn-1-yl and n-but-3-yn-1-yl.

In the context of the invention, (C₃-C₈)-cycloalkyl and (C₃-C₆)-cycloalkyl represent a monocyclic cycloalkyl group having 3 to 8 and 3 to 6 carbon atoms, respectively. Preference is given to a cycloalkyl radical having 3 to 6 carbon atoms. The following radicals may be mentioned by way of example and by way of preference: cyclopropyl, cyclobutyl, cyclopentyl, cyclohexyl and cycloheptyl.

In the context of the invention, (C₆-C₁₀)-aryl represents an aromatic radical having preferably 6 to 10 carbon atoms. Preferred aryl radicals are phenyl and naphthyl.

In the context of the invention, (C₁-C₆)-alkoxy and (C₁-C₄)-alkoxy represent a straight-chain or branched alkoxy radical having 1 to 6 and 1 to 4 carbon atoms, respectively. Preference is given to a straight-chain or branched alkoxy radical having 1 to 4 carbon atoms. The following radicals may be mentioned by way of example and by way of preference: methoxy, ethoxy, n-propoxy, isopropoxy and tert-butoxy.

In the context of the invention, (C₁-C₆-alkoxy carbonyl and (C₁-C₄)-alkoxy carbonyl represent a straight-chain or branched alkoxy radical having 1 to 6 and 1 to 4 carbon atoms, respectively, which is attached via a carbonyl group. Preference is given to a straight-chain or branched alkoxycarbonyl radical having 1 to 4 carbon atoms in the alkoxy group. The following radicals may be mentioned by way of example and by way of preference: methoxycarbonyl, ethoxycarbonyl, n-propoxycarbonyl, isopropoxycarbonyl and tert-butoxycarbonyl.

In the context of the invention, (C₁-C₆)-alkylsulphonyl represents a straight-chain or branched alkylsulphonyl radical having 1 to 6 carbon atoms. Preference is given to a straight-chain or branched alkylsulphonyl radical having 1 to 4 carbon atoms. The following radicals may be mentioned by way of example and by way of preference: methylsulphonyl, ethylsulphonyl, n-propylsulphonyl, isopropylsulphonyl, n-butylsulphonyl and tert-butylsulphonyl.

In the context of the invention, mono-(C₁-C₆)-alkylamino and mono-(C₁-C₄)-alkylamino represent an amino group having a straight-chain or branched alkyl substituent which has 1 to 6 and 1 to 4 carbon atoms, respectively. Preference is given to a straight-chain or branched monoalkylamino radical having 1 to 4 carbon atoms. The following radicals may be mentioned by way of example and by way of preference: methylamino, ethylamino, n-propylamino, isopropylamino and tert-butylamino.

In the context of the invention, di-(C₁-C₆)-alkylamino and di-(C₁-C₄)-alkylamino represent an amino group having two identical or different straight-chain or branched alkyl substituents which have in each case 1 to 6 and 1 to 4 carbon atoms, respectively. Preference is given to straight-chain or branched dialkylamino radicals having in each case 1 to 4 carbon atoms. The following radicals may be mentioned by way of example and by way of preference: N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

In the context of the invention, (C₁-C₆)-alkoxycarbonylamino and (C₁-C₄)-alkoxycarbonylamino represent an amino group having a straight-chain or branched alkoxycarbonyl substituent which has 1 to 6 and 1 to 4 carbon atoms, respectively, in the alkoxy radical and which is attached via the carbonyl group to the nitrogen atom. Preference is given to an alkoxycarbonylamino radical having 1 to 4 carbon atoms. The following radicals may be mentioned by way of example and by way of preference: methoxycarbonylamino, ethoxycarbonylamino, n-propoxycarbonylamino, isopropoxycarbonylamino and tert-butoxycarbonylamino.

In the context of the invention, (C₁-C₆)-alkanoyloxy and (C₁-C₄)-alkanoyloxy represent a straight-chain or branched alkyl radical having 1 to 6 and 1 to 4 carbon atoms, respectively, which carries a doubly attached oxygen atom in the 1-position and is attached in the 1-position via a further oxygen atom. Preference is given to an alkanoyloxy radical having 1 to 4 carbon atoms. The following radicals may be mentioned by way of example and by way of preference: acetoxy, propionoxy, n-butyroxy, isobutyroxy, pivaloyloxy and n-hexanoyloxy.

In the context of the invention, 5- to 10-membered heteroaryl represents a mono- or, if appropriate, bicyclic aromatic heterocyclic (heteroaromatic) having up to four identical or different heteroatoms from the group consisting of N, O and S, which radical is attached via a ring carbon atom or, if appropriate, via a ring nitrogen atom of the heteroatomatic. The following radicals may be mentioned by way of example: furyl, pyrrolyl, thienyl, pyrazolyl, imidazolyl, thiazolyl, oxazolyl, isoxazolyl, isothiazolyl, triazolyl, oxadiazolyl, thiadiazolyl, tetrazolyl, pyridyl, pyrimidinyl, pyridazinyl, pyrazinyl, triazinyl, benzofuranyl, benzothienyl, benzimidazolyl, benzoxazolyl, benzothiazolyl, benzotriazolyl, indolyl, indazolyl, quinolinyl, isoquinolinyl, naphthyridinyl, quinazolinyl, quinoxalinyl. Preference is given to monocyclic 5- or 6-membered heteroaryl radicals having up to three heteroatoms from the group consisting of N, O and S, such as, for example, furyl, thienyl, thiazolyl, oxazolyl, isothiazolyl, isoxazolyl, pyrazolyl, imidazolyl, triazolyl, oxadiazolyl, thiadiazolyl, pyridyl, pyrimidinyl, pyridazinyl, pyrazinyl, triazinyl.

In the context of the invention, halogen includes fluorine, chlorine, bromine and iodine. Preference is given to chlorine or fluorine.

If radicals in the compounds according to the invention are substituted, the radicals can, unless specified otherwise, be mono- or polysubstituted. In the context of the present invention, the meanings of all radicals which occur more than once are independent of one another. Substitution with one, two or three identical or different substituents is preferred. Very particular preference is given to substitution with one substituent.

In the context of the present invention, preference is given to compounds of the formula (I), in which
W, X, Y and Z together with the carbon atom to which Y and Z are attached form a 5-membered heteroaryl ring which may optionally be mono- or disubstituted by identical or different substituents from the group consisting of (C₁-C₆)-alkyl and trifluoromethyl and in which
   W represents C or N
   and
   X, Y and Z each represent C, N, O or S,
   where at least one of the ring members W, X, Y and Z represents a heteroatom from the group consisting of N, O and S,
A, in the case that W represents C, represents a bond or represents CH₂, C(═O), O, S or NR⁸, in which
   R⁸ represents hydrogen or (C₁-C₆)-alkyl,
   and,
   in the case that W represents N, represents a bond or represents CH₂ or C(═O),
R¹ represents (C₆-C₁₀)-aryl or 5- to 10-membered heteroaryl which may in each case be substituted up to four times by identical or different substituents selected from the group consisting of halogen, nitro, cyano, (C₁-C₆)-alkyl, (C₃-C₈)-cycloalkyl, phenyl, hydroxyl, (C₁-C₆)-alkoxy, trifluoromethyl, trifluoromethoxy, amino, mono- and di-(C₁-C₆)-alkyl-amino, R⁹—C(O)—NH—, R¹⁰—C(O)—, R¹¹R¹²N—C(O)—NH— and R¹³R¹⁴N—C(O)—, in which
   R⁹ represents hydrogen, (C₁-C₆)-alkyl, (C₃-C₈)-cycloalkyl, phenyl or (C₁-C₆)-alkoxy,
   R¹⁰ represents hydrogen, (C₁-C₆)-alkyl, (C₃-C₈)-cycloalkyl, phenyl, hydroxyl or (C₁-C₆)-alkoxy
   and
   R¹¹, R¹², R¹³ and R¹⁴ are identical or different and independently of one another represent hydrogen, (C₁-C₆)-alkyl, (C₃-C₈)-cycloalkyl or phenyl,
R² represents hydrogen, (C₆-C₁₀)-aryl, (C₁-C₆)-alkyl, (C₂-C₆)-alkenyl or (C₂-C₆)-alkynyl, in which alkyl, alkenyl and alkynyl may in each case be substituted by trifluoromethyl, fluorine, cyano, (C₁-C₆)-alkoxy, trifluoromethoxy, (C₆-C₁₀)-aryl or 5- or 6-membered heteroaryl, where all aryl and heteroaryl groups mentioned for their part may in each case be substituted up to three times by identical or different substituents selected from the group consisting of halogen, nitro, cyano, (C₁-C₆)-alkyl, hydroxyl, (C₁-C₆)-alkoxy, trifluoromethyl and trifluoromethoxy,
R³ and R⁴ are identical or different and independently of one another represent hydrogen, (C₁-C₆)-alkyl, (C₂-C₆)-alkenyl, (C₁-C₆)-alkoxy, trifluoromethyl, trifluoromethoxy or halogen,
R⁵ and R⁶ are identical or different and independently of one another represent hydrogen, (C₁-C₆)-alkyl, (C₁-C₆)-alkoxy or phenoxy or together with the carbon atom to which they are attached form a (C₃-C₈)-cycloalkyl ring,
and
R⁷ represents a group of the formula —NHR¹⁵ or —OR⁶, in which
   R¹⁵ represents hydrogen or (C₁-C₆)-alkyl
   and
   R¹⁶ represents hydrogen or represents a hydrolyzable group which can be converted into the corresponding carboxylic acid,
      and their salts, solvates and solvates of the salts.

In the context of the present invention, particular preference is given to compounds of the formula (I) in which
W, X, Y and Z together with the carbon atom to which Y and Z are attached form a 5-membered heteroaryl ring which may optionally be mono- or disubstituted by identical or different substituents from the group consisting of (C₁-C₄)-alkyl and trifluoromethyl and in which
   W represents C or N
   and
   X, Y and Z each represent C, N, O or S,
   where at least one of the ring members W, X, Y and Z represents N and at least one further of the ring members W, X, Y and Z represents a heteroatom from the group consisting of N, O and S,
A, in the case that W represents C, represents a bond or represents CH₂, C(═O), O, S or NR⁸, in which
   R⁸ represents hydrogen or (C₁-C₄)-alkyl,
   and,
   in the case that W represents N, represents a bond or represents CH₂ or C(═O),
R¹ represents phenyl or 5- or 6-membered heteroaryl which may in each case be substituted up to four times by identical or different substituents selected from the group consisting of halogen, nitro, cyano, (C₁-C₄)-alkyl, (C₃-C₆)-cycloalkyl, phenyl, hydroxyl, (C₁-C₄)-alkoxy, trifluoromethyl, trifluoromethoxy, amino, mono- and di-(C₁-C₄)-alkylamino, R⁹—C(O)—NH—, R¹⁰—C(O)—, R¹¹R¹²N—C(O)—NH— and R¹³R¹⁴N—C(O)—, in which
   R⁹ represents hydrogen, (C₁-C₄)-alkyl, (C₃-C₆)-cycloalkyl, phenyl or (C₁-C₄)-alkoxy,
   R¹⁰ represents hydrogen, (C₁-C₄)-alkyl, (C₃-C₆)-cycloalkyl, phenyl, hydroxyl or (C₁-C₄)-alkoxy
   and
   R¹¹, R¹², R¹³ and R¹⁴ are identical or different and independently of one another represent hydrogen, (C₁-C₄)-alkyl, (C₃-C₆)-cycloalkyl or phenyl,
R² represents hydrogen, phenyl, (C₁-C₄)-alkyl, (C₂-C₄)-alkenyl or (C₂-C₄)-alkynyl, in which alkyl, alkenyl and alkynyl may in each case be substituted by trifluoromethyl, fluorine, cyano, (C₁-C₄)-alkoxy, phenyl or 5- or 6-membered heteroaryl, where all phenyl and heteroaryl groups mentioned for their part may in each case be substituted up to three times by identical or different substituents selected from the group consisting of halogen, nitro, cyano, (C₁-C₄)-alkyl, hydroxyl, (C₁-C₄)-alkoxy, trifluoromethyl and trifluoromethoxy,
R³ and R⁴ are identical or different and independently of one another represent hydrogen, (C₁-C₄)-alkyl, (C₁-C₄)-alkoxy, trifluoromethyl, trifluoromethoxy or halogen,
R⁵ and R⁶ are identical or different and independently of one another represent hydrogen, methyl, ethyl, methoxy, ethoxy or phenoxy or together with the carbon atom to which they are attached form a (C₃-C₆)-cycloalkyl ring,
and
R⁷ represents a group of the formula —NHR¹⁵ or —OR¹⁶, in which
   R¹⁵ represents hydrogen or (C₁-C₄)-alkyl
   and
   R¹⁶ represents hydrogen or represents a hydrolyzable group which can be converted into the corresponding carboxylic acid,
      and their salts, solvates and solvates of the salts.

In the context of the present invention, very particular preference is given to compounds of the formula (I) in which
W, X, Y and Z together with the carbon atom to which Y and Z are attached form a 5-membered heteroaryl ring of the formula

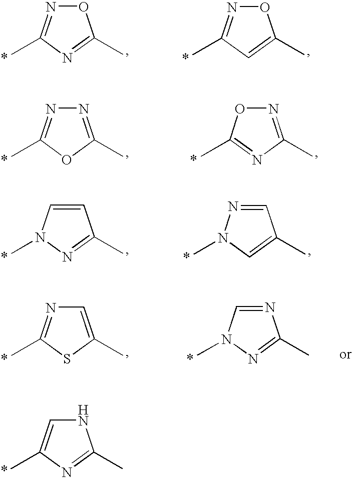

which may optionally be mono- or disubstituted by identical or different substituents from the group consisting of methyl and trifluoromethyl and in which * denotes the point of attachment to the group R¹-A-,
A, in the case that W represents C, represents a bond or represents CH₂, C(═O) or O
   and,
   in the case that W represents N, represents a bond or represents CH₂,
R¹ represents phenyl or pyridyl which may in each case be mono- or disubstituted by identical or different substituents selected from the group consisting of fluorine, chlorine, nitro, methyl, methoxy, trifluoromethyl and trifluoromethoxy,
R² represents hydrogen, propargyl or represents (C₁-C₄)-alkyl which may be substituted by fluorine, cyano, (C₁-C₄)-alkoxy, phenyl, furyl, thienyl, oxazolyl or thiazolyl, where phenyl and all heteroaromatic rings mentioned for their part may in each case be mono- or disubstituted by identical or different substituents selected from the group consisting of fluorine, chlorine, methyl, methoxy, trifluoromethyl and trifluoromethoxy,
R³ and R⁴ are identical or different and independently of one another represent hydrogen, methyl, methoxy, fluorine or chlorine,
R⁵ and R⁶ are identical or different and represent hydrogen or methyl,
and
R⁷ represents —OH, —NH₂ or —NHCH₃,
   and their salts, solvates and solvates of the salts.

In the context of the present invention, very particular preference is also given to compounds of the formula (I), in which
W, X, Y and Z together with the carbon atom to which Y and Z are attached form a 5-membered heteroaryl ring of the formula

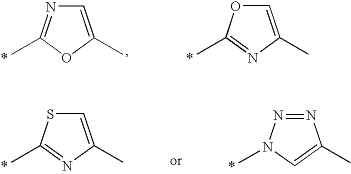

which may optionally be mono- or disubstituted by identical or different substituents from the group consisting of methyl and trifluoromethyl and in which * denotes the point of attachment to the group R¹-A-,
A, in the case that W represents C, represents a bond, CH₂ or O
   and,
   in the case that W represents N, represents a bond or represents CH₂,
R¹ represents phenyl which may be mono- or disubstituted by identical or different substituents selected from the group consisting of fluorine, chlorine, nitro, methyl, methoxy, trifluoromethyl and trifluoromethoxy,
R² represents (C₁-C₄)-alkyl, (C₂-C₄)-alkenyl or (C₂-C₄)-alkynyl which may in each case be substituted by fluorine, cyano, (C₁-C₄)-alkoxy, phenyl, furyl, thienyl, oxazolyl or thiazolyl, where phenyl and all heteroaromatic rings mentioned for their part may in each case be mono- or disubstituted by identical or different substituents selected from the group consisting of fluorine, chlorine, methyl, methoxy, trifluoromethyl and trifluoromethoxy,
R³ and R⁴ are identical or different and independently of one another represent hydrogen, methyl, methoxy, fluorine or chlorine,
R⁵ and R⁶ are identical or different and represent hydrogen or methyl,
and
R⁷ represents —OH, —NH₂ or —NHCH₃,
   and their salts, solvates and solvates of the salts.

In the context of the present invention, very particular preference is also given to compounds of the formula (I), in which A represents a bond or represents CH₂.

Of particular importance are compounds of the formula (I-A)

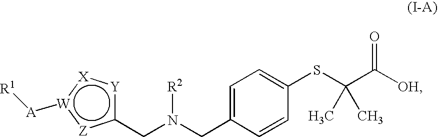

in which
A, W, X, Y, Z, R¹ and R² are each as defined above.

The individual radical definitions given in the respective combinations of preferred combinations of radicals may, independently of the particular given combination of radicals, also be replaced by radical definitions of other combinations.

Very particular preference is given to combinations of two or more of the preferred ranges mentioned above.

The invention furthermore provides a process for preparing the compounds of the formula (I) or (I-A) according to the invention, characterized in that compounds of the formula (II)

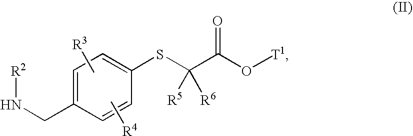

in which R², R³, R⁴, R⁵ and R⁶ are each as defined above
and
T¹ represents (C₁-C₄)-alkyl, preferably tert-butyl, or represents benzyl,
are initially reacted, in an inert solvent in the presence of a base, with a compound of the formula (III)

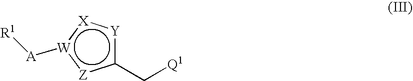

in which A, W, X, Y, Z and R¹ are each as defined above
and
Q¹ represents a suitable leaving group, such as, for example, halogen, mesylate, tosylate or triflate,
   to give compounds of the formula (I-B)

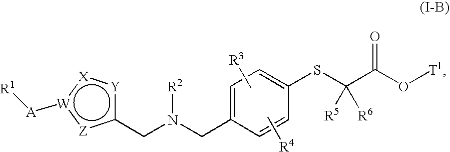

in which A, W, X, Y, Z, T¹, R¹, R², R³, R⁴, R⁵ and R⁶ are each as defined above,
these are then converted, by basic or acidic hydrolysis or, in the case that T¹ represents benzyl, also hydrogenolytically, into carboxylic acids of the formula (I-C)

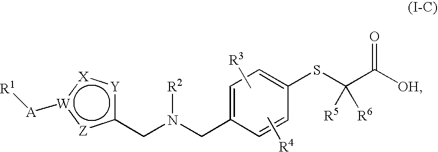

in which A, W, X, Y, Z, R¹, R², R³, R⁴, R⁵ and R⁶ are each as defined above,
and, if appropriate, subsequently converted by esterification or amidation methods known from the literature into the compounds of the formula (I)
and the compounds of the formula (I) are, if appropriate, using the appropriate (i) solvents and/or (ii) bases or acids, converted into their solvates, salts and/or solvates of the salts.

### Compounds of the Formula (I-D)

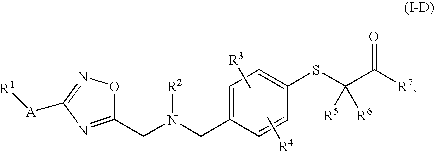

in which A, R¹, R², R³, R⁴, R⁵, R⁶ and R⁷ are each as defined above can also be prepared by initially converting compounds of the formula (II) in an inert solvent in the presence of a base with a compound of the formula (IV)

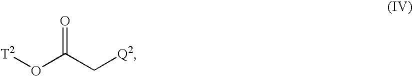

in which
T² represents (C₁-C₄)-alkyl, preferably methyl or ethyl,
and
Q² represents a suitable leaving group, such as, for example, halogen, mesylate, tosylate or triflate,
   into compounds of the formula (V)

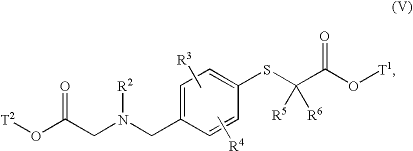

in which T¹, T², R², R³, R⁴, R⁵ and R⁶ are each as defined as above,
subsequently, under suitable reaction conditions, hydrolyzing selectively to carboxylic acids of the formula (VI)

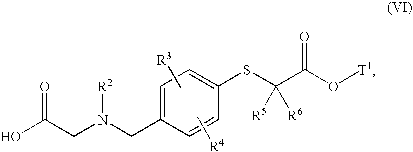

in which T¹, R², R³, R⁴, R⁵ and R⁶ are each as defined above,
then, in an inert solvent in the presence of a condensing agent, converting with a compound of the formula (VII)

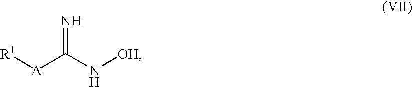

in which A and R¹ are each as defined above,
into compounds of the formula (VIII)

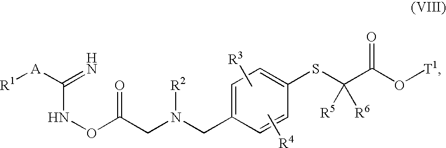

in which A, T¹, R¹, R², R³, R⁴, R⁵ and R⁶ are each as defined above,
then, with or without intermediate isolation, cyclizing these in the presence of a base to compounds of the formula (I-E)

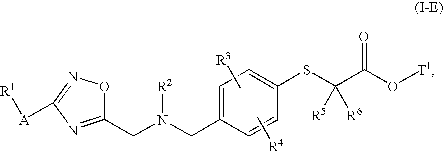

in which A, T¹, R¹, R², R³, R⁴, R⁵ and R⁶ are each as defined above,
then, by basic or acidic hydrolysis or, in the case that T¹ represents benzyl, also hydrogenolytically, converting these into carboxylic acids of the formula (I-F)

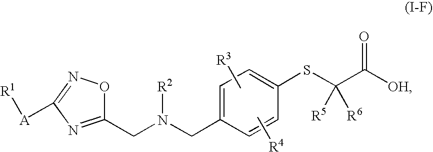

in which A, R¹, R², R³, R⁴, R⁵ and R⁶ are each as defined above,
and, if appropriate, finally converting by esterification or amidation methods known from the literature into the compounds of the formula (I-D).

### Compounds of the Formula (I-G)

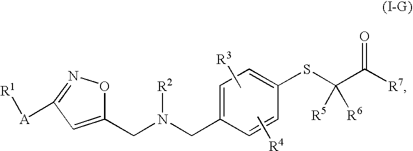

in which A represents a bond and R¹, R², R³, R⁴, R⁵, R⁶ and R⁷ are each as defined above can also be prepared by initially converting compounds of the formula (II) in an inert solvent in the presence of a base with a compound of the formula (IX)

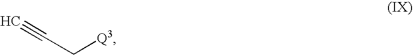

### HC Q, (IX),

in which
Q³ represents a suitable leaving group, such as, for example, chlorine, bromine or iodine, into compounds of the formula (X)

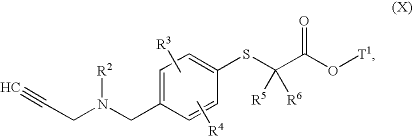

### R_{0 R}⁴

in which T¹, R², R³, R⁴, R⁵ and R⁶ are each as defined above,
then in an inert solvent in the presence of N-chlorosuccinimide and a base, with a compound of the formula (XI)

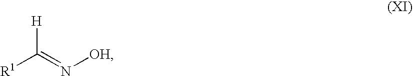

### H R1N OH(XI),

in which R¹ is as defined above
via a 1,3-dipolar cycloaddition into compounds of the formula (I-H)

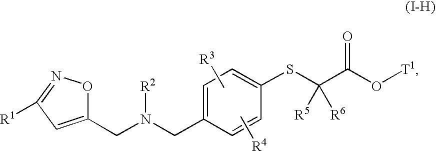

0
in which T¹, R¹, R², R³, R⁴, R⁵ and R⁶ are each as defined above,
then, by basic or acidic hydrolysis or, in the case that T¹ represents benzyl, also hydrogenolytically converting these into carboxylic acids of the formula (I-K)

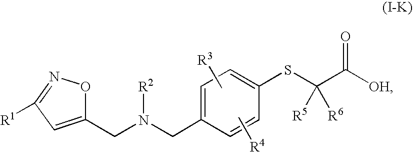

in which R¹, R², R³, R⁴, R⁵ and R⁶ are each as defined above,
and, if appropriate, finally converting these by esterification or amidation methods known from the literature into the compounds of the formula (I-G).

### Compounds of the Formula (I-L)

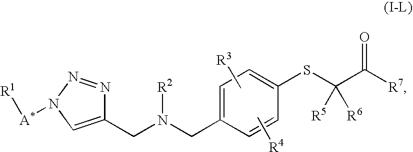

in which A* represents a CH₂ group or represents a bond and R¹, R², R³, R⁴, R⁵, R⁶ and R⁷ are each as defined above can also be prepared by converting compounds of the formula (X)

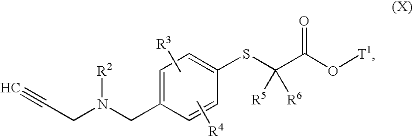

in which T¹, R², R³, R⁴, R⁵ and R⁶ are each as defined above
in an inert solvent in the presence of a copper(I) catalyst with an azide of the formula (XVI)

R¹-A*-N₃ (XVI),

in which R¹ is as defined above
and
A* represents a bond or represents a CH₂ group,
via a 1,3-dipolar cycloaddition into compounds of the formula (I-M)

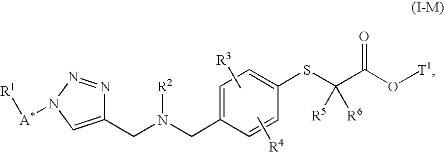

in which A*, T¹, R¹, R², R³, R⁴, R⁵ and R⁶ are each as defined above,
then converting these by basic or acidic hydrolysis into carboxylic acids of the formula (I-N)

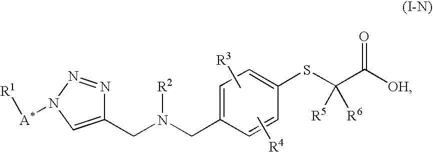

in which A*, R¹, R², R³, R⁴, R⁵ and R⁶ are each as defined above,
and, if appropriate, finally converting these by esterification or amidation methods known from the literature into the compounds of the formula (I-L).

Inert solvents for the process steps (II)+(III)→(I-B), (II)+(IV)→(V) and (II)+(IX)→(X) are, for example, halogenated hydrocarbons, such as dichloromethane, trichloromethane, carbon tetrachloride, trichloroethane, tetrachloroethane, 1,2-dichloroethane or trichloroethylene, ethers, such as diethyl ether, dioxane, tetrahydrofuran, glycol dimethyl ether or diethylene glycol dimethyl ether, hydrocarbons, such as benzene, xylene, toluene, hexane, cyclohexane or mineral oil fractions, or other solvents, such as ethyl acetate, acetone, dimethylformamide, dimethyl sulphoxide, N,N′-dimethylpropyleneurea (DMPU), N-methylpyrrolidone (NMP), pyridine or acetonitrile. It is also possible to use mixtures of the solvents mentioned. Preference is given to tetrahydrofuran and dimethylformamide.

Suitable bases for the process steps (II)+(III)→(I-B), (II)+(IV)→(V) and (II)+(IX)→(X) are the customary inorganic or organic bases. These preferably include alkali metal hydroxides, such as, for example, lithium hydroxide, sodium hydroxide or potassium hydroxide, alkali metal or alkaline earth metal carbonates, such as lithium carbonate, sodium carbonate, potassium carbonate, calcium carbonate or caesium carbonate, alkali metal alkoxides, such as sodium methoxide or potassium methoxide, sodium ethoxide or potassium ethoxide or potassium tert-butoxide, alkali metal hydrides, such as sodium hydride, amides, such as sodium amide, lithium bis(trimethylsilyl)amide or potassium bis(trimethylsilyl)amide or lithium diisopropylamide, or organic amines, such as triethylamine, N-methylmorpholine, N-methylpiperidine, N,N-diisopropylethylamine, pyridine, 1,5-diazabicyclo[4.3.0]non-5-ene (DBN), 1,4-diazabicyclo[2.2.2]octane (DABCO®) or 1,8-diazabicyclo[5.4.0]undec-7-ene (DBU). Preferred for the process step (II)+(III)→(I-B) is N,N-diisopropylethylamine, preferred for the process step (II)+(IV)→(V) is triethylamine or caesium carbonate, and preferred for the process step (II)+(IX)→(X) is caesium carbonate.

In these process steps, the base is in each case employed in an amount of from 1 to 5 mol, preferably in an amount of from 1 to 2.5 mol, based on 1 mol of the compound of the formula (II) or its hydrochloride. The reaction is generally carried out in a temperature range of from 0° C. to +100° C., preferably from +20° C. to +80° C. The reaction can be carried out at atmospheric, elevated or reduced pressure (for example from 0.5 to 5 bar). In general, the reaction is carried out at atmospheric pressure.

The hydrolysis of the carboxylic esters in the process steps (I-B)→(I-C), (V)→(VI), (I-E)→(I-F) and (I-H)→(I-K) is carried out by customary methods by treating the esters in inert solvents with bases, where the salts initially formed are converted by treatment with acid into the free carboxylic acids. In the case of the tert-butyl esters, ester cleavage is preferably carried out using acids.

Suitable inert solvents for the hydrolysis of the carboxylic acids are water or the organic solvents customary for ester cleavage. These preferably include alcohols, such as methanol, ethanol, n-propanol, isopropanol, n-butanol or tert-butanol, or ethers, such as diethyl ether, tetrahydrofuran, dioxane or glycol dimethyl ether, or other solvents, such as acetone, acetonitrile, dichloromethane, dimethyl formamide or dimethyl sulphoxide. It is also possible to use mixtures of the solvents mentioned. In the case of a basic ester hydrolysis, preference is given to using mixtures of water with dioxane, tetrahydrofuran, methanol and/or ethanol. In the case of reaction with trifluoroacetic acid, preference is given to using dichloromethane, and in the case of the reaction with hydrogen chloride, preference is given to using tetrahydrofuran, diethyl ether, dioxane or water.

Suitable bases for the ester hydrolysis are the customary inorganic bases. These preferably include alkali metal or alkaline earth metal hydroxides, such as, for example, sodium hydroxide, lithium hydroxide, potassium hydroxide or barium hydroxide, or alkali metal or alkaline earth metal carbonates, such as sodium carbonate, potassium carbonate or calcium carbonate. Particular preference is given to using sodium hydroxide or lithium hydroxide.

Suitable acids for the ester cleavage are, in general, sulphuric acid, hydrogen chloride/hydrochloric acid, hydrogen bromide/hydrobromic acid, phosphoric acid, acetic acid, trifluoroacetic acid, toluenesulphonic acid, methanesulphonic acid or trifluoromethanesulphonic acid, or mixtures thereof, if appropriate with addition of water. Preference is given to hydrogen chloride or trifluoroacetic acid in the case of the tert-butyl esters and to hydrochloric acid in the case of the methyl esters.

The ester cleavage is generally carried out in a temperature range of from −20° C. to +100° C., preferably from 0° C. to +50° C. The reaction can be carried out at atmospheric, elevated or reduced pressure (for example from 0.5 to 5 bar). In general, the reaction is carried out at atmospheric pressure.

The process steps (I-C)→(I), (I-F)→(I-D), (I-K)→(I-G) and (VI)+(VII)→(VIII) are carried out by methods known from the literature for esterifying or amidating (amide formation) of carboxylic acids.

Inert solvents for these process steps are, for example, ethers, such as diethyl ether, dioxane, tetrahydrofuran, glycol dimethyl ether or diethylene glycol dimethyl ether, hydrocarbons, such as benzene, toluene, xylene, hexane, cyclohexane or mineral oil fractions, halogenated hydrocarbons, such as dichloromethane, trichloromethane, carbon tetrachloride, 1,2-dichloroethane, trichloroethylene or chlorobenzene, or other solvents, such as ethyl acetate, pyridine, dimethyl sulphoxide, dimethylformamide, N,N′-dimethylpropyleneurea (DMPU), N-methylpyrrolidone (NMP), acetonitrile or acetone. It is also possible to use mixtures of the solvents mentioned. Preference is given to dichloromethane, tetrahydrofuran, dimethylformamide or mixtures of these solvents.

Suitable condensing agents for an esterification or amide formation in the process steps (I-C)→(I), (I-F)→(I-D), (I-K)→(I-G) or (VI)+(VII)→(VIII) are, for example, carbodiimides, such as N,N′-diethyl-, N,N′-dipropyl-, N,N′-diisopropyl-, N,N′-dicyclohexylcarbodiimide (DCC), N-(3-dimethylaminoisopropyl)-N′-ethylcarbodiimide hydrochloride (EDC), or phosgene derivatives, such as N,N′-carbonyldiimidazole, or 1,2-oxazolium compounds, such as 2-ethyl-5-phenyl-1,2-oxazolium 3-sulphate or 2-tert-butyl-5-methyl-isoxazolium perchlorate, or acylamino compounds, such as 2-ethoxy-1-ethoxycarbonyl-1,2-dihydroquinoline, or isobutyl chloroformate, propanephosphonic anhydride, diethyl cyanophosphonate, bis(2-oxo-3-oxazolidinyl)phosphoryl chloride, benzotriazol-1-yloxytris(dimethylamino)phosphonium hexafluorophosphate, benzo-triazol-1-yloxytris(pyrrolidino)phosphonium hexafluorophosphate (PyBOP), O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetra-methyluronium hexafluorophosphate (HATU), if appropriate in combination with further auxiliaries, such as 1-hydroxybenzotriazole (HOBt) or N-hydroxysuccinimide (HOSu), and also, as bases, alkali metal carbonates, for example sodium carbonate or potassium carbonate or sodium bicarbonate or potassium bicarbonate, or organic bases, such as trialkylamines, for example triethylamine, N-methylmorpholine, N-methylpiperidine or N,N-diisopropylethylamine. For the process steps (I-C)→(I), (I-F)→(I-D) and (I-K)→(I-G), preference is given to using PyBOP in combination with N,N-diisopropylethylamine. For process step (VI)+(VII)→(VIII), preference is given to using N,N′-diisopropylcarbodiimide in combination with HOBt.

The process steps (I-C)→(I), (I-F)→(I-D), (I-K)→(I-G) and (VI)+(VII)→(VIII) are generally carried out in a temperature range of from −20° C. to +60° C., preferably from −10° C. to +40° C. The reaction can be carried out at atmospheric, elevated or reduced pressure (for example 0.5 to 5 bar). In general, the reaction is carried out at atmospheric pressure.

The cyclization in process step (VIII)→(I-E) is preferably carried out in the presence of a base, in particular sodium acetate, in an alcoholic solvent, in particular ethanol, at elevated temperature, in particular in a temperature range of from +50° C. to +80° C.

In the 1,3-dipolar cycloaddition in process step (X)+(XI)→(I-H), the nitrile oxide derived from the aldoxime (XI) is prepared in situ by reacting (XI) with N-chlorosuccinimide and a catalytic amount of pyridine (conversion into the corresponding N-hydroxylimidoyl chloride) and subsequent reaction with triethylamine in the presence of the acetylene component (X) [cf. K.-C. Liu, B. R. Shelton, R. K. Howe, J. Org. Chem. 45, 3916 (1980); M. Christl, R. Huisgen, Chem. Ber. 106, 3345 (1973); P. Caramella, P. Grunanger, in 1,3-Dipolar cycloaddition Chemistry, A. Padwa, Ed., Wiley, New York, 1984]. The process step is preferably carried out in chloroform in a temperature range of from +20° C. to +60° C.

In the 1,3-dipolar cycloaddition in process step (X)+(XVI)→(I-M), the azide of the formula (XVI) can also be prepared in situ by reacting a corresponding halide with sodium azide. The catalyst used is preferably the system copper(II) sulphate/sodium ascorbate [cf. A. K. Feldman et al., Org. Lett. 6 (22), 3897-3899 (2004)]. The process step is preferably carried out in dimethyl-formamide, dimethyl sulphoxide or mixtures thereof with water, in a temperature range of from +20° C. to +80° C.

The compounds of the formula (II) and their preparation are described in WO 02/28821, or they can be prepared analogously to the processes described therein. Compounds of the formula (II) in which R² represents hydrogen can also be prepared by converting compounds of the formula (XII)

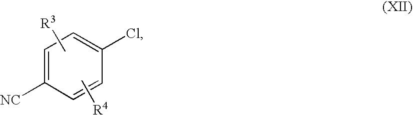

in which R³ and R⁴ are each as defined above,
initially in an inert solvent with sodium sulphide into compounds of the formula (XIII)

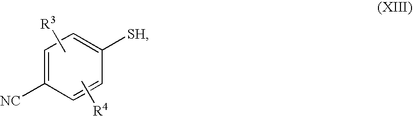

in which R³ and R⁴ are each as defined above,
converting these subsequently with or without intermediate isolation with a compound of the formula (XIV)

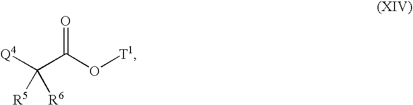

in which T¹, R⁵ and R⁶ are each as defined above,
and
Q⁴ represents a suitable leaving group, such as, for example, halogen, mesylate, tosylate or triflate,
   into compounds of the formula (XV)

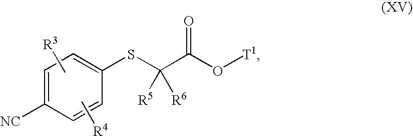

in which T¹, R³, R⁴, R⁵ and R⁶ are each as defined above,
followed by reduction with a suitable reducing agent, such as, preferably borane or borane complexes (for example diethylaniline, dimethyl sulphide or tetrahydrofuran complexes) or else with sodium borohydride in combination with aluminium chloride.

The compounds of the formulae (IV), (VII), (IX), (XI), (XII), (XIV) and (XVI) are commercially available, known from the literature or can be prepared analogously to processes known from the literature.

Some of the compounds of the formula (III) are commercially available, known from the literature or can be prepared by methods known from the literature. This is illustrated in an exemplary manner in the synthesis schemes A-E below:

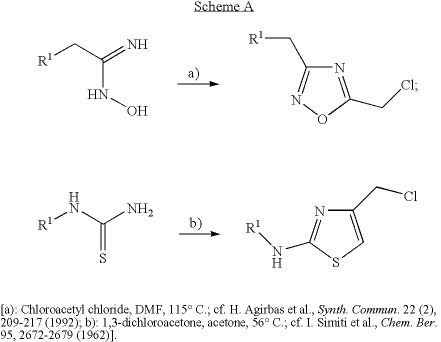

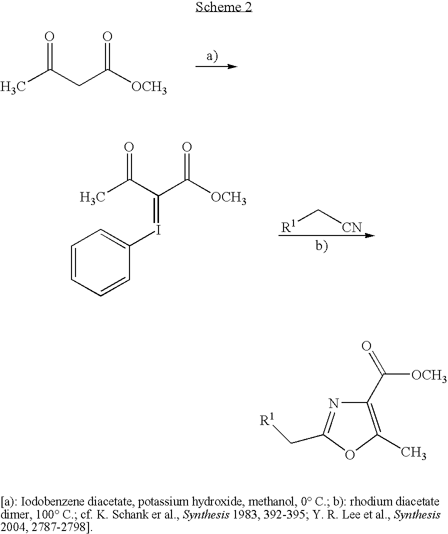

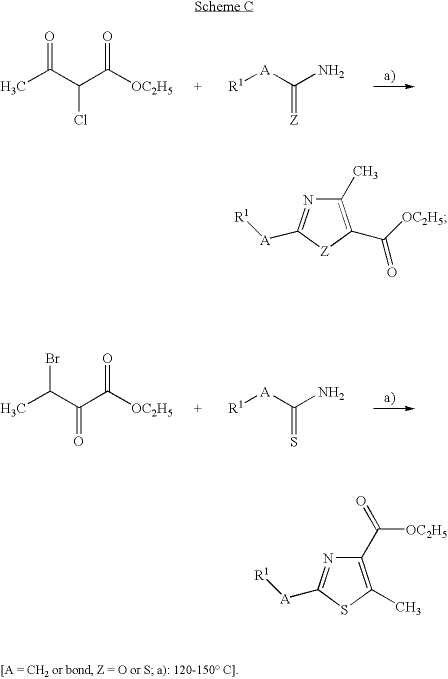

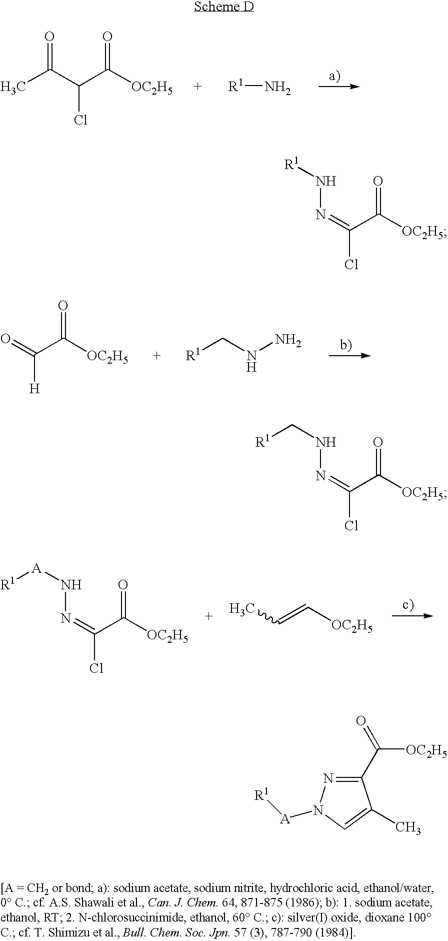

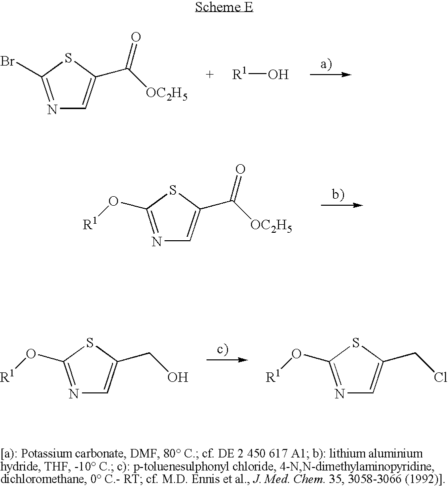

The heteroarylcarboxylic esters obtained according to process schemes B, C and D are converted analogously to the reaction sequence described in scheme E via a reduction with lithium aluminium hydride and subsequent reaction with p-toluenesulphonyl chloride/4-N,N-dimethyl-aminopyridine into the corresponding chloromethyl derivatives according to formula (III).

The preparation of the compounds according to the invention can be illustrated by synthesis schemes 1-4:

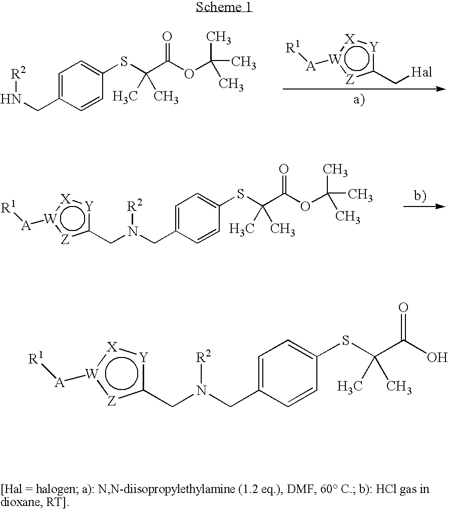

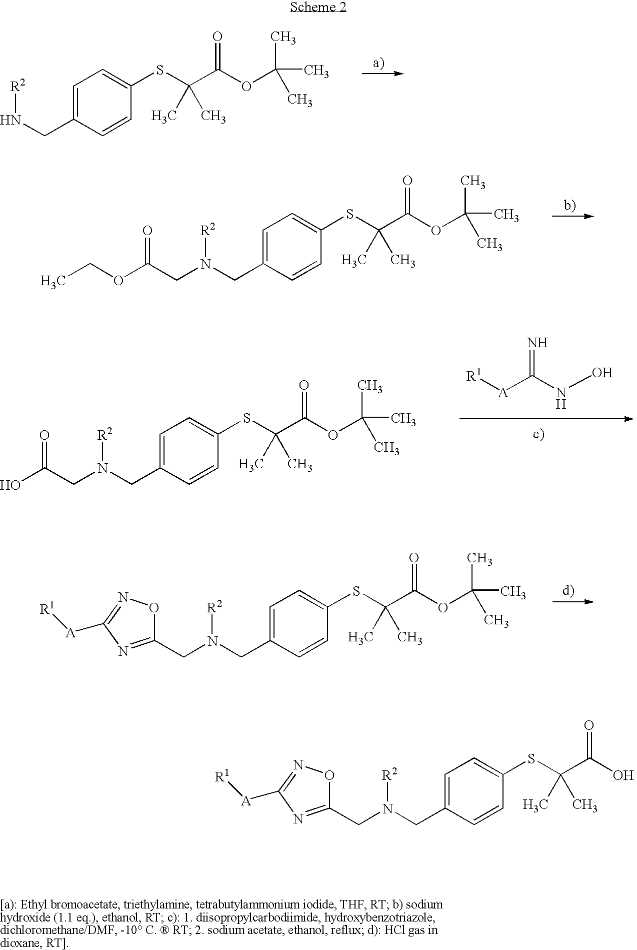

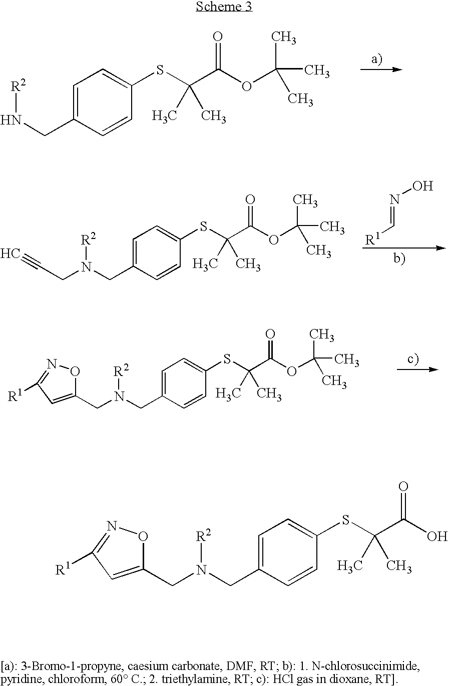

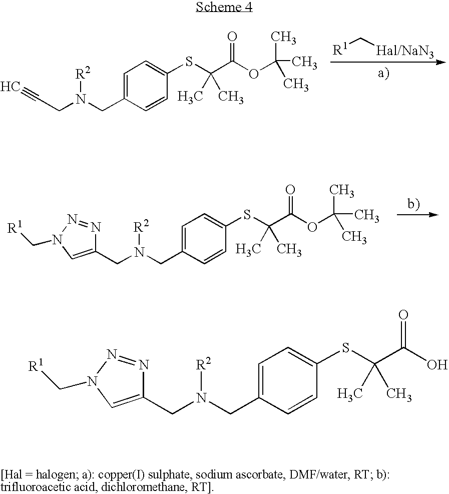

The compounds according to the invention have useful pharmacological properties and can be used for the prevention and treatment of disorders in humans and animals.

The compounds according to the invention are highly effective PPAR-alpha modulators and as such are suitable in particular for the primary and/or secondary prevention and treatment of cardiovascular disorders caused by disturbances in fatty acid and glucose metabolism. Such disorders include dyslipidaemias (hypercholesterolaemia, hypertriglyceridaemia, elevated concentrations of postprandial plasma triglycerides, hypoalphalipoproteinaemia, combined hyperlipidaemias), arteriosclerosis and metabolic disorders (metabolic syndrome, hyperglycaemia, insulin-dependent diabetes, non-insulin-dependent diabetes, gestation diabetes, hyperinsulinaemia, insulin resistance, glucose intolerance, obesity (adipositas) and late sequelae of diabetes, such as retinopathy, nephropathy and neuropathy).

Further independent risk factors for cardiovascular disorders which can be treated by the compounds according to the invention are high blood pressure, ischaemia, myocardial infaction, angina pectoris, cardiac insufficiency, myocardial insufficiency, restenosis, elevated levels of fibrinogen and of LDL of low density and also elevated concentrations of plasminogen activator inhibitor 1 (PAI-1).

In addition, the compounds according to the invention can also be used for the treatment and/or prevention of micro- and macrovascular damage (vasculitis), reperfusion damage, arterial and venous thromboses, oedema, cancerous disorders (skin cancer, liposarcomas, carcinomas of the gastrointestinal tract, of the liver, of the pancreas, of the lung, of the kidney, of the urethra, of the prostate and of the genital tract), of disorders of the central nervous system and neurodegenerative disorders (strokes, Alzheimer's disease, Parkinson's disease, dementia, epilepsy, depression, multiple sclerosis), of inflammatory disorders, immune disorders (Crohn's disease, ulcerative colitis, lupus erythematodes, rheumatoid arthritis, asthma), renal disorders (glomerulonephritis), disorders of the thyroid gland, disorders of the pancreas (pancreatitis), fibrosis of the liver, skin disorders (psoriasis, acne, eczema, neurodermitis, dermatitis, keratitis, formation of scars, formation of warts, frostbites), viral diseases (HPV, HCMV, HIV), cachexia, osteoporosis, gout, incontinence, and also for wound healing and angiogenesis.

The activity of the compounds according to the invention can be examined, for example, in vitro by the transactivation assay described in the experimental section.

The in vivo activity of the compounds according to the invention can be examined, for example, by the tests described in the experimental section.

The present invention furthermore provides the use of the compounds according to the invention for the treatment and/or prevention of disorders, in particular the disorders mentioned above.

The present invention also provides the use of the compounds according to the invention for preparing a medicament for the treatment and/or prevention of disorders, in particular the disorders mentioned above.

The present invention also provides a method for the treatment and/or prevention of disorders, in particular the disorders mentioned above, using an effective amount of at least one compound according to the invention.

The compounds according to the invention can be used alone or, if required, in combination with other active compounds. The present invention furthermore provides medicaments comprising at least one compound according to the invention and one or more further active compounds, in particular for the treatment and/or prevention of the disorders mentioned above.

Suitable active compounds for combinations are, by way of example and by way of preference: substances which modulate lipid metabolism, antidiabetics, hypotensive agents, perfusion-enhancing and/or antithrombotic agents and also antioxidants, chemokine receptor antagonists, p38-kinase inhibitors, NPY agonists, orexin agonists, anorectics, PAF-AH inhibitors, antiphlogistics (COX inhibitors, LTB₄-receptor antagonists), analgesics (aspirin), antidepressants and other psychopharmaceuticals.

The present invention provides in particular combinations comprising at least one of the compounds according to the invention and at least one lipid metabolism-modulating active compound, an antidiabetic, a hypotensive compound and/or an antithrombotic agent.

Preferably, the compounds according to the invention can be combined with one or more
lipid metabolism-modulating active compounds, by way of example and by way of preference from the group of the HMG-CoA reductase inhibitors, inhibitors of HMG-CoA reductase expression, squalene synthesis inhibitors, ACAT inhibitors, LDL receptor inductors, cholesterol absorption inhibitors, polymeric bile acid adsorbers, bile acid reabsorption inhibitors, MTP inhibitors, lipase inhibitors, LpL activators, fibrates, niacin, CETP inhibitors, PPAR-γ and/or PPAR-δ agonists, RXR modulators, FXR modulators, LXR modulators, thyroid hormones and/or thyroid mimetics, ATP citrate lyase inhibitors, Lp(a) antagonists, cannabinoid receptor 1 antagonists, leptin receptor agonists, bombesin receptor agonists, histamine receptor agonists and the antioxidants/radical scavengers,
antidiabetics mentioned in the Rote Liste 2004/II, chapter 12, and also, by way of example and by way of preference, those from the group of the sulphonylureas, biguanides, meglitinide derivatives, glucosidase inhibitors, oxadiazolidinones, thiazolidinediones, GLP 1 receptor agonists, glucagon antagonists, insulin sensitizers, CCK 1 receptor agonists, leptin receptor agonists, inhibitors of liver enzymes involved in the stimulation of gluconeogenesis and/or glycogenolysis, modulators of glucose uptake and also potassium channel openers, such as, for example, those disclosed in WO 97/26265 and WO 99/03861,
hypotensive compounds, by way of example and by way of preference from the group of the calcium antagonists, angiotensin AII antagonists, ACE inhibitors, beta-receptor blockers, alpha-receptor blockers, diuretics, phosphodiesterase inhibitors, sGC stimulators, cGMP level elevating substances, aldosterone antagonists, mineralocorticoid receptor antagonists, ECE inhibitors and the vasopeptidase inhibitors, and/or
antithrombotic agents, by way of example and by way of preference from the group of the platelet aggregation inhibitors or the anticoagulants.

Lipid metabolism-modifying active compounds are to be understood as meaning, preferably, compounds from the group of the HMG-CoA reductase inhibitors, squalene synthesis inhibitors, ACAT inhibitors, cholesterol absorption inhibitor, MTP inhibitors, lipase inhibitors, thyroid hormones and/or thyroid mimetics, niacin receptor agonists, CETP inhibitors, PPAR-gamma agonists, PPAR-delta agonists, polymeric bile acid adsorbers, bile acid reabsorption inhibitors, antioxidants/radical scavengers and also the cannabinoid receptor 1 antagonists.

In a preferred embodiment of the invention, the compounds according to the invention are administered in combination with an HMG-CoA reductase inhibitor from the class of the statins, such as, by way of example and by way of preference, lovastatin, simvastatin, pravastatin, fluvastatin, atorvastatin, rosuvastatin, cerivastatin or pitavastatin.

In a preferred embodiment of the invention, the compounds according to the invention are administered in combination with a squalene synthesis inhibitor, such as, by way of example and by way of preference, BMS-188494 or TAK-475.

In a preferred embodiment of the invention, the compounds according to the invention are administered in combination with an ACAT inhibitor, such as, by way of example and by way of preference, melinamide, pactimibe, eflucimibe or SMP-797.

In a preferred embodiment of the invention, the compounds according to the invenetion are administered in combination with a cholesterol absorption inhibitor, such as, by way of example and by way of preference, ezetimibe, tiqueside or pamaqueside.

In a preferred embodiment of the invention, the compounds according to the invention are administered in combination with an MTP inhibitor, such as, by way of example and by way of preference, implitapide or JTT-130.

In a preferred embodiment of the invention, the compounds according to the invention are administered in combination with a lipase inhibitor, such as, by way of example and by way of preference, orlistat.

In a preferred embodiment of the invention, the compounds according to the invention are administered in combination with a thyroid hormone and/or thyroid mimetic, such as, by way of example and by way of preference, D-thyroxine or 3,5,3′-triiodothyronine (T3).

In a preferred embodiment of the invention, the compounds according to the invention are administered in combination with an agonist of the niacin receptor, such as, by way of example and by way of preference, niacin, acipimox, acifran or radecol.

In a preferred embodiment of the invention, the compounds according to the invention are administered in combination with a CETP inhibitor, such as, by way of example and by way of preference, torcetrapib, JTT-705 or CETP vaccine (Avant).

In a preferred embodiment of the invention, the compounds according to the invention are administered in combination with a PPAR-gamma agonist, such as, by way of example and by way of preference, pioglitazone or rosiglitazone.

In a preferred embodiment of the invention, the compounds according to the invention are administered in combination with a PPAR-delta agonist, such as, by way of example and by way of preference, GW-501516.

In a preferred embodiment of the invention, the compounds according to the invention are administered in combination with a polymeric bile acid adsorber, such as, by way of example and by way of preference, cholestyramine, colestipol, colesolvam, CholestaGel or colestimide.

In a preferred embodiment of the invention, the compounds according to the invention are administered in combination with a bile acid reabsorption inhibitor, such as, by way of example and by way of preference, ASBT (=IBAT) inhibitors, such as, for example, AZD-7806, S-8921, AK-105, BARI-1741, SC435 or SC-635.

In a preferred embodiment of the invention, the compounds according to the invention are administered in combination with a antioxidant/radical scavenger, such as, by way of example and by way of preference, probucol, AGI-1067, BO-653 or AEOL-10150.

In a preferred embodiment of the invention, the compounds according to the invention are administered in combination with a cannabinoid receptor 1 antagonist, such as, by way of example and by way of preference, rimonabant or SR-147778.

Antidiabetics are to be understood as meaning, preferably, insulin and insulin derivatives, and also orally effective hypoglycaemic acid compounds. Here, insulin and insulin derivatives include both insulins of animal, human or biotechnological origin and also mixtures thereof. The orally effective hypoglycaemic active compounds preferably include sulphonylureas, biguanides, meglitinide derivatives, glucosidase inhibitors and PPAR-gamma agonists.

In a preferred embodiment of the invention, the compounds according to the invention are administered in combination with insulin.

In a preferred embodiment of the invention, the compounds according to the invention are administered in combination with a sulphonylurea, such as, by way of example and by way of preference, tolbutamide, glibenclamide, glimepiride, glipizide or gliclazide.

In a preferred embodiment of the invention, the compounds according to the invention are administered in combination with a biguanide, such as, by way of example and by way of preference, metform.

In a preferred embodiment of the invention, the compounds according to the invention are administered in combination with a meglitinide derivative, such as, by way of example and by way of preference, repaglinide or nateglinide.

In a preferred embodiment of the invention, the compounds according to the invention are administered in combination with a glucosidase inhibitor, such as, by way of example and by way of preference, miglitol or acarbose.

In a preferred embodiment of the invention, the compounds according to the invention are administered in combination with a PPAR-gamma agonist, for example from the class of the thiazolidinediones, such as, by way of example and by way of preference, pioglitazone or rosiglitazone.

The hypotensive agents are preferably understood as meaning compounds from the group of the calcium antagonists, angiotensin AII antagonists, ACE inhibitors, beta-receptor blockers, alpha-receptor blockers and diuretics.

In a preferred embodiment of the invention, the compounds according to the invention are administered in combination with a calcium antagonist, such as, by way of example and by way of preference, nifedipine, amlodipine, verapamil or diltiazem.

In a preferred embodiment of the invention, the compounds according to the invention are administered in combination with an angiotensin AII antagonist, such as, by way of example and by way of preference, losartan, valsartan, candesartan, embusartan or telmisartan.

In a preferred embodiment of the invention, the compounds according to the invention are administered in combination with an ACE inhibitor, such as, by way of example and by way of preference, enalapril, captopril, ramipril, delapril, fosinopril, quinopril, perindopril or trandopril.

In a preferred embodiment of the invention, the compounds according to the invention are administered in combination with a beta-receptor blocker, such as, by way of example and by way of preference, propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol oder bucindolol.

In a preferred embodiment of the invention, the compounds according to the invention are administered in combination with an alpha-receptor blocker, such as, by way of example and by way of preference, prazosin.

In a preferred embodiment of the invention, the compounds according to the invention are administered in combination with a diuretic, such as, by way of example and by way of preference, furosemide.

In a preferred embodiment of the invention, the compounds according to the invention are administered in combination with antisympathotonics, such as reserpine, clonidine or alpha-methyldopa, with potassium channel-agonists, such as minoxidil, diazoxide, dihydralazine or hydralazine, or with nitrous oxide-releasing compounds, such as glycerol nitrate or sodium nitroprusside.

Antithrombotics are to be understood as meaning, preferably, compounds from the group of the platelet aggregation inhibitors or the anticoagulants.

In a preferred embodiment of the invention, the compounds according to the invention are administered in combination with a platelet aggregation inhibitor, such as, by way of example and by way of preference, aspirin, clopidogrel, ticlopidine or dipyridamol.

In a preferred embodiment of the invention, the compounds according to the invention are administered in combination with a thrombin inhibitor, such as, by way of example and by way of preference, ximelagatran, melagatran, bivalirudin or clexane.

In a preferred embodiment of the invention, the compounds according to the invention are administered in combination with a GPIIb/IIIa antagonist, such as, by way of example and by way of preference, tirofiban or abciximab.

In a preferred embodiment of the invention, the compounds according to the invention are administered in combination with a factor Xa inhibitor, such as, by way of example and by way of preference, DX-9065a, DPC 906, JTV 803, BAY 59-7939, DU-176b, fidexaban, razaxaban, fondaparinux, idraparinux, PMD-3112, YM-150, KFA-1982, EMD-503982, MCM-17, MLN-1021, SSR-126512 or SSR-128428.

In a preferred embodiment of the invention, the compounds according to the invention are administered in combination with heparin or a low molecular weight (LMW) heparin derivative.

In a preferred embodiment of the invention, the compounds according to the invention are administered in combination with a vitamin K antagonist, such as, by way of example and by way of preference, coumarin.

The compounds according to the invention can act systemically and/or locally. For this purpose, they can be administered in a suitable manner, such as, for example, orally, parenterally, pulmonally, nasally, sublingually, lingually, buccally, rectally, dermally, transdermally, conjunctivally, otically or as an implant or stent.

For these administration routes, the compounds according to the invention can be administered in suitable administration forms.

Suitable for oral administration are administration forms which work in accordance with the prior art and release the compounds according to the invention rapidly and/or in modified form and which comprise the compounds according to the invention in crystalline and/or amorphicized and/or dissolved form, such as, for example, tablets (uncoated or coated tablets, for example with enteric coats or coats which dissolve in a delayed manner or are insoluble and which control the release of the compounds according to the invention), films/wafers or tablets which dissolve rapidly in the oral cavity, filmsayophilizates, capsules (for example hard or soft gelatin capsules), sugar-coated tablets, granules, pellets, powders, emulsions, suspensions, aerosols or solutions.

Parenteral administration may take place by circumventing a bioabsorption step (for example intravenously, intraarterially, intracardially, intraspinally or intralumbarly), or with bioabsorption (for example intramuscularly, subcutaneously, intracutaneously, percutaneously or intraperitoneally). Administration forms suitable for parenteral administration are inter alia preparations for injection or infusion in the form of solutions, suspensions, emulsions, lyophilizates or sterile powders.

Suitable for other administration routes are, for example, medicaments suitable for inhalation (inter alia powder inhalers, nebulizers), nose drops, solutions or sprays, tablets to be administered lingually, sublingually or buccally, films/wafers or capsules, suppositories, preparations to be administered to ears or eyes, vaginal capsules, aqueous suspensions (lotions, shaking mixtures), lipophilic suspensions, ointments, creams, transdermal therapeutic systems (for example plasters), milk, pastes, foams, powders for pouring, implants or stents.

Preference is given to oral or parenteral administration, in particular to oral administration.

The compounds according to the invention can be converted into the administration forms mentioned. This can be carried out in a manner known per se by mixing with inert non-toxic pharmaceutically suitable auxiliaries. These auxiliaries include inter alia carriers (for example microcrystalline cellulose, lactose, mannitol), solvents (for example liquid polyethylene glycols), emulsifiers and dispersants or wetting agents (for example sodium dodecyl sulphate, polyoxysorbitan oleate), binders (for example polyvinylpyrrolidone), synthetic and natural polymers (for example albumin), stabilizers (for example antioxidants, such as, for example, ascorbic acid), colorants (for example inorganic pigments, such as, for example, iron oxides), and flavour and/or odour corrigents.

The present invention furthermore provides medicaments comprising at least one compound according to the invention, usually together with one or more inert non-toxic pharmaceutically suitable auxiliaries, and their use for the purposes mentioned above.

In general, it has been found to be advantageous in the case of parenteral administration to administer amounts of about 0.001 to 1 mg/kg, preferably about 0.01 to 0.5 mg/kg of body weight to obtain effective results. In the case of oral administration, the dosage is from about 0.01 to 100 mg/kg, preferably from about 0.01 to 20 mg/kg and very particularly preferably from 0.1 to 10 mg/kg of body weight.

In spite of this, it may be necessary to deviate from the amounts mentioned, namely depending on body weight, administration route, individual response to the active compound, the type of preparation and the time or the interval at which administration takes place. Thus, in some cases it may be sufficient to administer less than the abovementioned minimum amount, whereas in other cases the upper limit mentioned has to be exceeded. In the case of the administration of relatively large amounts, it may be expedient to divide these into a plurality of individual doses which are administered over the course of the day.

The working examples below illustrate the invention. The invention is not limited to the examples.

The percentages in the tests and examples below are, unless indicated otherwise, percentages by weight; parts are parts by weight. Solvent ratios, dilution ratios and concentrations of liquid/liquid solutions are in each case based on volume.

### A. EXAMPLES

### Abbreviations

TLC thin-layer chromatography
DCI direct chemical ionization (in MS)
DCM dichloromethane
DMF dimethylformamide
DMSO dimethyl sulphoxide
eq. equivalent(s)
ESI electrospray ionization (in MS)
CC gas chromatography
h hour(s)
HPLC high-pressure, high-performance liquid chromatography
LC/MS liquid chromatography-coupled mass spectroscopy
min minute(s)
MS mass spectroscopy
MTBE methyl tert-butyl ether
NMP N-methylpyrrolidone
NMR nuclear magnetic resonance spectroscopy
PyBOP benzotriazol-1-yloxytris(pyrrolidino)phosphonium hexafluorophosphate
RT room temperature
Rₜ retention time (in HPLC)
THF tetrahydrofuran
UV ultraviolet spectroscopy

### LC/MS, HPLC and GC Methods:

### Method 1 (HPLC):

Instrument: HP 1100 with DAD detection; column: Kromasil 100 RP-18, 60 mm×2.1 mm, 3.5 μm; mobile phase A: 5 ml of HClO₄ (70% strength)/ 1 of water, mobile phase B: acetonitrile; gradient: 0 m in 2% B→0.5 min 2% B→4.5 min 90% B→9 min 90% B→9.2 min 2% B→10 min 2% B; flow rate: 0.75 ml/min; column temperature: 30° C.; detection: UV 210 nm.

### Method 2 (LC/MS):

MS instrument type: Micromass ZQ; HPLC instrument type: Waters Alliance 2795; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

### Method 3 (LC/MS):

Instrument: Micromass Quattro LCZ with HPLC Agilent series 1100; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 208-400 nm.

### Method 4 (LC/MS):

Instrument: Micromass Platform LCZ with HPLC Agilent series 1100; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

### Method 5 (LC/MS):

MS instrument type: Micromass ZQ; HPLC instrument type: HP 1100 series; UV DAD; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

### Method 6 (LC/MS):

Instrument: Micromass Platform LCZ with HPLC Agilent series 1100; column: Thermo HyPURITY Aquastar 3μ 50 mm×2.1 mm; mobile phase A: 1 l of water+0.5 ml of 50% strength formic acid, mobile phase B: 1 l of acetonitrile+0.5 ml of 50% strength formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

### Method 7 (HPLC)

Instrument: HP 1100 with DAD detection; column: Kromasil 100 RP-18, 60 mm×2.1 mm, 3.5 μm; mobile phase A: 5 ml of HClO₄ (70% strength)/l of water, mobile phase B: acetonitrile; gradient: 0 min 2% B→0.5 min 2% B→4.5 min 90% B→6.5 min 90% B→6.7 min 2% B 7.5 min 2% B; flow rate: 0.75 ml/min; column temperature: 30° C.; detection: UV 210 nm.

### Method 8 (GC):

Instrument: HP 5890 with FID detector; injector temperature: 200° C.; detector temperature: 310° C.; column: HP5, fused silica, 5% phenylmethylsiloxane, length: 25 m, internal diameter: 0.2 mm, film thickness: 0.33 μm; column pre-pressure: 100 kPa; split valve: 100 ml/min; carrier gas: hydrogen; gas for flushing: nitrogen; analysis programme: start at 50° C., then heating rate 10° C./min, final temperature 300° C., holding time 20 min, stop after 45 min; test solution: about 50 mg of the sample in 2 ml of dichloromethane; injection volume: 1.0 μl.

### Method 9 (LC/MS):

MS instrument type: Micromass ZQ; HPLC instrument type: Waters Alliance 2795; column: Merck Chromolith SpeedROD RP-18e 50 mm×4.6 mm; mobile phase A: water+500 μl of 50% strength formic acid/1; mobile phase B: acetonitrile+500 μl of 50% strength formic acid/1; gradient: 0.0 min 10% B→3.0 min 95% B→4.0 min 95% B; oven: 35° C.; flow rate: 0.0 min 1.0 ml/min→3.0 min 3.0 ml/min→4.0 min 3.0 ml/min; UV detection: 210 nm.

### Starting Materials and Intermediates:

### Example 1A

### N-Hydroxy-2,4-dimethylbenzamidine

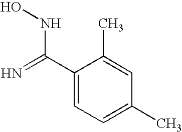

5.00 g of 2,4-dimethylbenzonitrile are dissolved in 10.5 ml of ethanol, a 50% strength solution of hydroxylamine in water is added and the mixture is heated under reflux for 1 day. The reaction mixture is cooled to room temperature, whereupon the target compound precipitates out. The product is filtered off and dried under high vacuum. This gives 2.61 g (41% of theory) of the title compound.

¹H-NMR (400 MHz, CDCl₃): δ=2.32 (s, 3H), 2.40 (s, 3H), 4.74 (br. s, 2H), 6.99-7.04 (m, 2H), 7.28 (br. s, 1H).

LC/MS (method 6): Rₜ=1.58 min; MS (ESIpos): m/z=165 [M+H]⁺.

### Example 2A

### tert-Butyl 2-[(4-{[(2-furylmethyl)(2-ethoxy-2-oxoethyl)amino]methyl}phenyl)thio]-2-methyl-propionate

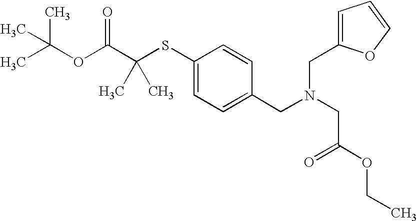

3.00 g of tert-butyl 2-[(4-{[(2-furylmethyl)amino]methyl}phenyl)thio]-2-methylpropionate hydrochloride (7.46 mmol) [WO 02/28821, Example II-3] are suspended in 30 ml of DMF, and 4.86 g of caesium carbonate (14.91 mmol) and 1.25 g of ethyl bromoacetate (7.46 mmol) are added. The reaction mixture is stirred at room temperature overnight. 100 ml of water are added and the mixture is extracted three times with dichloromethane. The combined organic phases are dried over sodium sulphate and the solvent is removed on a rotary evaporator. The residue is purified by chromatography on silica gel (mobile phase: cyclohexane/ethyl acetate 10:1). This gives 1.87 g (56% of theory) of the title compound.

¹H-NMR (400 MHz, CDCl₃): δ=1.26 (t, J=7.2, 3H), 1.41 (s, 9H), 1.43 (s, 6H), 3.32 (s, 2H), 3.80 (s, 2H), 3.84 (s, 2H), 4.16 (q, J=7.2, 2H), 6.19-6.20 (m, 1H), 6.31 (dd, J=3.0, J=1.9, 1H), 7.32-7.35 (m, 2H), 7.38 (dd, J=1.9, J=0.8, 1H), 7.44-7.47 (m, 2H).

LC/MS (method 2): Rₜ=3.06 min; MS (ESIpos): m/z=448 [M+H]⁺.

### Example 3A

### N-{4-[(2-tert-Butoxy-1,1-dimethyl-2-oxoethyl)thio]benzyl}-N-(2-furylmethyl)glycine

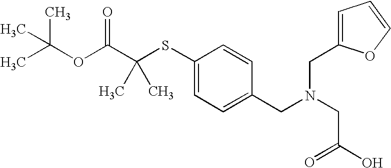

1.00 g of the compound from Example 2A (2.23 mmol) is dissolved in 7 ml of dioxane/water (2:1), and 3.37 ml of 1 N aqueous sodium hydroxide solution (3.37 mmol) are added. The reaction mixture is stirred at room temperature for 16 h. The mixture is acidified with 2 N hydrochloric acid (pH 2) and extracted three times with dichloromethane. The combined organic phases are dried over sodium sulphate and concentrated on a rotary evaporator. This gives 0.832 g (89% of theory) of the title compound.

¹H-NMR (400 MHz, CDCl₃): δ=1.42 (s, 9H), 1.44 (s, 6H), 3.32 (s, 2H), 3.76 (s, 2H), 3.77 (s, 2H), 6.26-6.27 (m, 1H), 6.35-6.36 (m, 1H), 7.26-7.28 (m, 2H), 7.43-7.44 (m, 1H), 7.49-7.51 (m, 2H).

LC/MS (method 2): Rₜ=1.95 min; MS (ESIpos): m/z=420 [M+H]⁺.

### Example 4A

### tert-Butyl 2-[(4-{[[2-({[(2,4-dimethylphenyl)(imino)methyl]amino}oxy)-2-oxoethyl](2-furyl-methyl)amino]methyl}phenyl)thio]-2-methylpropionate

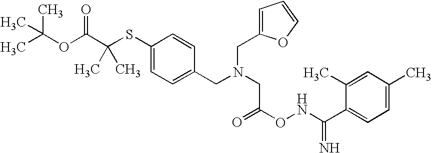

400 mg of the compound from Example 3A (0.95 mmol) and 188 mg of the compound from Example 1A (1.14 mmol) are dissolved in 6 ml of DCM/DMF (9:1), and 155 mg of 1-hydroxy-1H-benzotriazole (1.14 mmol) and 144 mg of N,N-diisopropylcarbodiimide (1.14 mmol) are added at −10° C. The mixture is stirred at −10° C. for 20 min and at room temperature for a further 1.5 h. The reaction mixture is concentrated on a rotary evaporator and the residue is taken up in ethyl acetate. The organic phase is washed with saturated sodium bicarbonate solution, with water and with 0.5 M potassium hydrogensulphate solution. The organic phase is dried over sodium sulphate and the solvent is removed on a rotary evaporator. This gives 669 mg (82% of theory) of the title compound which is used for the next step without further purification.

LC/MS (method 2): Rₜ=3.16 min; purity: 66% (UV 210 nm); MS (ESIpos): m/z=566 [M+H]⁺.

### Example 5A

### tert-Butyl 2-[(4-{[{[3-(2,4-dimethylphenyl)-1,2,4-oxadiazol-5-yl]methyl}(2-furylmethyl)amino]-methyl}phenyl)thio]-2-methylpropionate

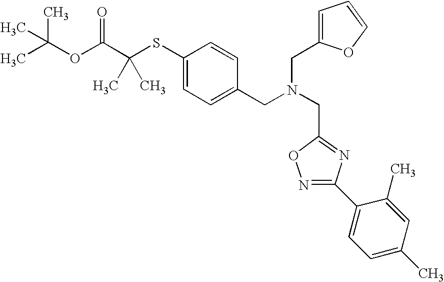

537 mg of the compound from Example 4A (0.63 mmol) are dissolved in 4.7 ml of ethanol, and a solution of 82 mg of sodium acetate (1.00 mol) in 0.7 ml of water is added. The solution is heated under reflux for 3 h. After cooling, water is added and the reaction mixture is extracted with ethyl acetate. The combined organic phases are dried over sodium sulphate and the solvent is removed on a rotary evaporator. The residue is purified by preparative HPLC (mobile phase: acetonitrile/water with 0.1% formic acid, gradient 20:80→95:5). This gives 275 mg (80% of theory) of the title compound.

¹H-NMR (400 MHz, CDCl₃): δ=1.41 (s, 9H), 1.43 (s, 6H), 2.38 (s, 3H), 2.61 (s, 3H), 3.83 (s, 2H), 3.86 (s, 2H), 4.00 (s, 2H), 6.28-6.29 (m, 1H), 6.33 (dd, J=3.2, J=1.9, 1H), 7.12-7.14 (m, 2H), 7.38-7.40 (m, 3H), 7.47-7.49 (m, 2H), 7.92 (d, J=7.9, 1H).

LC/MS (method 3): Rₜ=3.53 min; MS (ESIpos): m/z=548 [M+H]⁺.

### Example 6A

### tert-Butyl 2-{[4-({(2-furylmethyl)[(3-phenyl-1,2,4-oxadiazol-5-yl)methyl]amino}methyl)phenyl]-thio}-2-methylpropionate

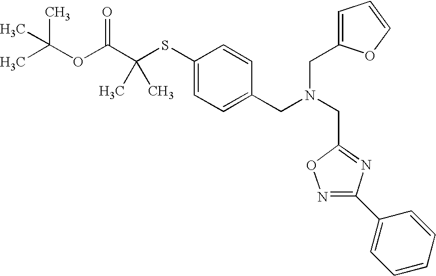

165 mg of tert-butyl 2-[(4-{[(2-furylmethyl)amino]methyl}phenyl)thio]-2-methylpropionate hydrochloride (0.41 mmol) [WO 02/28821, Example II-3] are dissolved in 2 ml of DMF, and 81 mg of 5-(chloromethyl)-3-phenyl-1,2,4-oxadiazole (0.41 mmol) and 118 mg of diisopropylethylamine (0.91 mmol) are added. The solution is stirred at room temperature for 16 h, and the reaction mixture is, without further work-up, purified directly by preparative HPLC (mobile phase: acetonitrile/water with 0.1% formic acid, gradient 20:80→95:5). This gives 169 mg (78% of theory) of the title compound.

¹H-NMR (400 MHz, CDCl₃): δ=1.41 (s, 9H), 1.43 (s, 6H), 3.82 (s, 2H), 3.85 (s, 2H), 4.00 (s, 2H), 6.28-6.29 (m, 1H), 6.33 (dd, J=3.2, J=1.9, 1H), 7.37-7.40 (m, 3H), 7.47-7.51 (m, 5H), 8.09-8.12 (m, 2H).

LC/MS (method 4): Rₜ=3.50 min; MS (ESIpos): m/z=520 [M+H]⁺.

### Example 7A

### 4-Methoxy-2-methylbenzaldehyde oxime

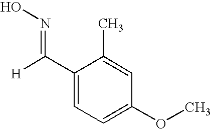

0.25 g of hydroxylamine hydrochloride (3.65 mmol) is dissolved in 5 ml of water, and 0.46 g of sodium bicarbonate (5.48 mmol) is added a little at a time. After 30 min of stirring at room temperature, 0.46 g of 4-methoxy-2-methylbenzaldehyde (3.05 mmol), dissolved in 5 ml of methanol, is added, and the mixture is stirred at room temperature for another 1.5 h. The reaction mixture is concentrated on a rotary evaporator and the aqueous residue is extracted three times with ethyl acetate. The combined organic phases are dried over sodium sulphate, the solvent is distilled off on a rotary evaporator and the residue is dried under high vacuum. This gives 0.62 g (73% of theory) of the title compound which is reacted further without further purification.

LC/MS (method 5): Rₜ=1.90 min; purity: 56% (UV 210 nm); MS (ESIpos): m/z=166 [M+H]⁺.

### Example 8A

### 2,4-Bis(trifluoromethyl)benzaldehyde oxime

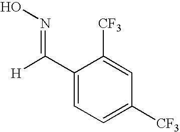

0.25 g of hydroxylamine hydrochloride (3.65 mmol) is dissolved in 5 ml of water, and 0.46 g of sodium bicarbonate (5.48 mmol) is added a little at a time. After 30 min of stirring at room temperature, 0.74 g of 2,4-bis(trifluoromethyl)benzaldehyde (3.05 mmol), dissolved in 5 ml of methanol, is added, and the mixture is stirred at room temperature for a further 1.5 h. The reaction mixture is concentrated on a rotary evaporator and the aqueous residue is extracted three times with ethyl acetate. The combined organic phases are dried over sodium sulphate, the solvent is distilled off on a rotary evaporator and the residue is dried under high vacuum. This gives 0.64 g (79% of theory) of the title compound.

LC/MS (method 2): Rₜ=2.35 min; purity: 96% (UV 210 nm); MS (ESIpos): m/z=256 [M+H]⁺.

### Example 9A

### tert-Butyl 2-[(4-{[(2-furylmethyl)(prop-2-yn-1-yl)amino]methyl}phenyl)thio]-2-methylpropionate

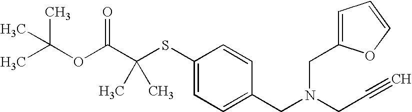

3.00 g of tert-butyl 2-[(4-{[(2-furylmethyl)amino]methyl}phenyl)thio]-2-methylpropionate hydrochloride (7.46 mmol) [WO 02/28821, Example H-3] are suspended in 30 ml of DMF, and 4.86 g of caesium carbonate (14.91 mmol) and 0.89 g of 3-bromo-1-propyne (7.46 mmol) are added. The reaction mixture is stirred at room temperature overnight. 100 ml of water are added, and the mixture is extracted three times with dichloromethane. The combined organic phases are dried over sodium sulphate and the solvent is removed on a rotary evaporator. The residue is purified by chromatography on silica gel (mobile phase: cyclohexane/ethyl acetate 12:1). This gives 1.76 g (59% of theory) of the title compound.

¹H-NMR (400 MHz, DMSO-d₆): δ=1.33 (s, 9H), 1.37 (s, 6H), 3.20 (s, 2H), 3.28 (s, 2H), 3.63-3.64 (m, 3H), 6.31-6.32 (m, 1H), 6.40 (dd, J=3.0, J=1.9, 1H), 7.32-7.34 (m, 2H), 7.41-7.44 (m, 2H), 7.59-7.60 (m, 1H).

LC/MS (method 2): Rₜ=3.06 min; MS (ESIpos): m/z=400 [M+H]⁺.

### Example 10A

### tert-Butyl 2-({4-[((2-furylmethyl) {[3-(4-methoxy-2-methylphenyl)isoxazol-5-yl]methyl}amino)-methyl]phenyl}thio)-2-methylpropionate

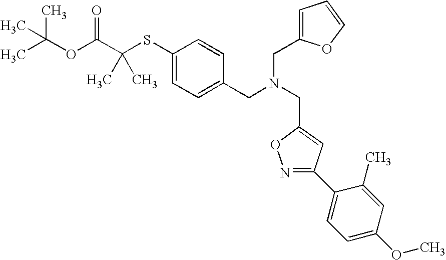

121 mg of 4-methoxy-2-methylbenzaldehyde oxime (Example 7A) (0.44 mmol) are dissolved in 1 ml of chloroform, 3 μl of pyridine (3 mg, 0.04 mmol) and 60 mg of N-chlorosuccinimide (0.44 mmol) are added and the mixture is stirred at 60° C. for 20 min. After cooling, 160 mg of the compound from Example 9A (0.40 mmol) and 61 mg of triethylamine (0.60 mmol), dissolved in 2 ml of chloroform, are added, and the reaction mixture is stirred at room temperature for 16 h. 2 ml of 0.5 N hydrochloric acid are added, the mixture is filtered through an Extrelut cartridge (Extrelut NT3, from Merck KGaA) and the filtrate is concentrated on a rotary evaporator. The residue is purified by preparative HPLC (mobile phase: acetonitrile/water with 0.1% formic acid, gradient 20:80→95:5). This gives 86 mg (38% of theory) of the title compound.

¹H-NMR (400 MHz, CDCl₃): δ=1.41 (s, 9H), 1.43 (s, 6H), 2.48 (s, 3H), 3.72 (s, 2H), 3.74 (s, 2H), 3.83 (s, 2H), 3.84 (s, 3H), 6.25-6.26 (m, 1H), 6.32-6.35 (m, 2H), 6.78-6.84 (m, 2H), 7.35-7.49 (m, 6H).

LC/MS (method 2): Rₜ=3.31 min; MS (ESIpos): m/z=563 [M+H]⁺.

### Example 11A

### tert-Butyl 2-[(4-{[({3-[2,4-bis(trifluoromethyl)phenyl]isoxazol-5-yl}methyl)(2-furylmethyl)-amino]methyl}phenyl)thio]-2-methylpropionate

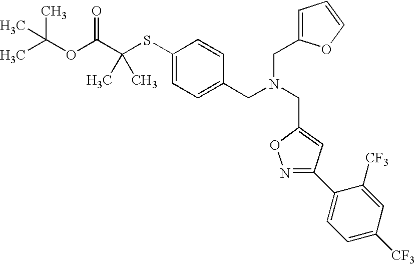

119 mg of 2,4-bis(trifluoromethyl)benzaldehyde oxime (Example 8A) (0.44 mmol) are dissolved in 1 ml of chloroform, 3 μl of pyridine (3 mg, 0.04 mmol) and 60 mg of N-chlorosuccinimide (0.44 mmol) are added and the mixture is stirred at 60° C. for 20 min. After cooling, 160 mg of the compound from Example 9A (0.40 mmol) and 61 mg of triethylamine (0.60 mmol), dissolved in 2 ml of chloroform, are added, and the reaction mixture is stirred at room temperature for 16 h. 2 ml of 0.5 N hydrochloric acid are added, the mixture is filtered through an Extrelut cartridge (Extrelut NT3, from Merck KGaA) and the filtrate is concentrated on a rotary evaporator. The residue is purified by preparative HPLC (mobile phase: acetonitrile/water with 0.1% of formic acid, gradient 20:80→95:5). This gives 45 mg (17% of theory) of the title compound.

¹H-NMR (400 MHz, CDCl₃): δ=1.41 (s, 9H), 1.44 (s, 6H), 3.72 (s, 2H), 3.74 (s, 2H), 3.86 (s, 2H), 6.24-6.25 (m, 1H), 6.35 (dd, J=3.0, J=1.9, 1H), 6.43 (br. s, 1H), 7.34-7.49 (m, 5H), 7.81-7.83 (m, 1H), 7.89-7.92 (m, 1H), 8.06 (br. s, 1H).

LC/MS (method 5): Rₜ=3.52 min; MS (ESIpos): m/z=655 [M+H]⁺.

### Example 12A

### tert-Butyl 2-[(4-{[(2-ethoxy-2-oxoethyl)(2-methoxyethyl)amino]methyl}phenyl)thio]-2-methylpropionate

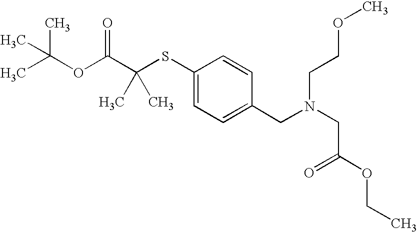

350 mg of tert-butyl 2-[[4-[[(2-methoxyethyl)amino]methyl]phenyl]thio]-2-methylpropionate (1.03 mmol) [WO 02/28821, Example II-9] in 5 ml of tetrahydrofuran are admixed with 172 μl of triethylamine (260 mg, 2.58 mmol), 190 mg of tetrabutylammonium iodide (0.514 mmol) and 359 μl of ethyl bromoacetate (259 mg, 1.55 mmol). The reaction mixture is stirred at room temperature overnight. 20 ml of water are added, and the mixture is extracted three times with in each case 20 ml of ethyl acetate. The combined organic phases are washed with 50 ml of water and 50 ml of saturated sodium chloride solution and then dried over sodium sulphate. After removal of the solvent under reduced pressure, the residue is purified by preparative HPLC (mobile phase: acetonitrile/water with 0.1% formic acid, gradient 10:90→95:5). This gives 276 mg (63% of theory) of the title compound.

¹H-NMR (400 MHz, DMSO-d₆): δ=1.18 (t, 3H), 1.33 (s, 9H), 1.36 (s, 6H), 2.75 (t, 2H), 3.18 (s, 3H), 3.37 (s, 2H), 3.38 (t, 2H), 3.79 (s, 2H), 4.07 (q, 2H), 7.33 (d, 2H), 7.41 (d, 2H).

MS (ESIpos): m/z=426 [M+H]⁺

HPLC (method 1): Rₜ=4.69 min

### Example 13A

### N-{4-[(2-tert-Butoxy-1,1-dimethyl-2-oxoethyl)thio]benzyl}-N-(2-methoxyethyl)glycine

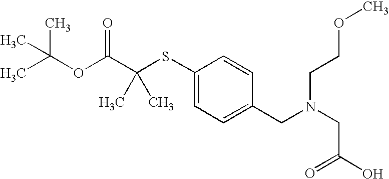

250 mg of the compound from Example 12A (0.587 mmol) are dissolved in 2 ml of ethanol, and 26 mg of sodium hydroxide (0.65 mmol) are added. The reaction mixture is stirred at room temperature overnight. 10 ml of water are added, and the mixture is extracted three times with in each case 10 ml of ethyl acetate. The aqueous phase is adjusted to pH 1 using 1 N hydrochloric acid and then extracted three times with in each case 10 ml of ethyl acetate. The organic phases are dried over magnesium sulphate and the solvent is removed under reduced pressure. The residue (132 mg) is reacted further without further purification.

LC/MS (method 3): Rₜ=1.89 min; MS (ESIneg): m/z=396 [M−H]⁺.

### Example 14A

### tert-Butyl 2-[(4-{[{[3-(2,4-dimethylphenyl)-1,2,4-oxadiazol-5-yl]methyl}(2-methoxyethyl)amino]-methyl}phenyl)thio]-2-methylpropionate

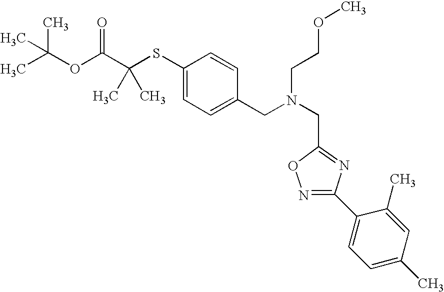

At −10° C., 49.0 mg of 1-hydroxy-1H-benzotriazole (0.362 mmol) and 45.7 mg of N,N-diisopropyl-carbodiimide (0.362 mmol) are added to 120 mg of the compound from Example 13A (0.288 mmol) and 59.5 mg of the compound from Example 1A (0.362 mmol) in 5 ml of dichloromethane/dimethylformamide (9:1). The mixture is stirred at −10° C. for 20 min and then at room temperature overnight. 15 ml of ethyl acetate are added to the reaction mixture. The mixture is then washed twice with saturated sodium bicarbonate solution, once with water, twice with 0.5 M of potassium hydrogensulphate solution and once with saturated sodium chloride solution (in each case 10 ml). The organic phases are dried over magnesium sulphate and the solvent is removed under reduced pressure. The residue is taken up in 5 ml of ethanol. 27.2 mg of sodium acetate (0.332 mmol) and 20 μl of water are added, and the mixture is then heated under reflux overnight. 10 ml of water are added and the mixture is extracted three times with in each case 10 ml of ethyl acetate. The organic phases are dried over magnesium sulphate and the solvent is removed under reduced pressure. The residue is purified by preparative HPLC (mobile phase: acetonitrile/water with 0.1% formic acid, gradient 20:80→95:5). This gives 47.7 mg (30% of theory) of the title compound.

¹H-NMR (400 MHz, DMSO-d₆): δ=1.33 (s, 9H), 1.36 (s, 6H), 2.35 (s, 3H), 2.52 (s, 3H), 2.79 (t, 2H), 3.19 (s, 3H), 3.47 (t, 2H), 3.83 (s, 2H), 4.11 (s, 2H), 7.19 (d, 1H), 7.23 (s, 1H), 7.40 (m, 4H), 7.82 (d, 1H).

MS (ESIpos): m/z=526 [M+H]⁺

HPLC (method 1): Rₜ=5.40 min

### Example 15A

### tert-Butyl 2-{[4-({(2-furylmethyl)[(5-phenyl-1,3,4-oxadiazol-2-yl)methyl]amino}methyl)phenyl]-thio}-2-methylpropionate

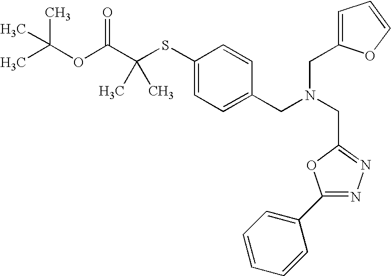

117 mg of 2-chloromethyl-5-phenyl-1,3,4-oxadiazole (0.603 mmol) [preparation, for example, according to B. Chai et al., Heterocycl. Commun. 8 (6), 601-606 (2002)] and 220 μl of N,N-diisopropylethylamine (162 mg, 1.26 mmol) are added to 200 mg of tert-butyl 2-[(4-{[(2-furylmethyl)amino]methyl}phenyl)thio]-2-methylpropionate (0.502 mmol) [WO 02/28821, Example II-3] in 2 ml of dimethylformamide. The mixture is stirred at 60° C. for 5 h and then purified directly by preparative HPLC (mobile phase: acetonitrile/water with 0.1% formic acid, gradient 20:80→95:5). This gives 190 mg (70% of theory) of the title compound.

¹H-NMR (300 MHz, DMSO-d₆): δ=1.32 (s, 9H), 1.35 (s, 6H), 3.77 (s, 2H), 3.79 (s, 2H), 3.97 (s, 2H), 6.34-6.37 (m, 1H), 6.39-6.42 (m, 1H), 7.37-7.45 (m, 4H), 7.57-7.65 (m, 4H), 7.95-8.01 (m, 2H).

MS (ESIpos): m/z=520 [M+H]⁺

HPLC (method 1): Rₜ=5.46 min

### Example 16A

### 2-(4-Fluorophenyl)-N-hydroxyethaneimideamide

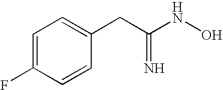

136 μl of hydroxylamine (146 mg, 4.44 mmol) are added to 200 mg of 4-fluorobenzyl cyanide (1.48 mmol) in 2 ml of ethanol, and the mixture is heated under reflux overnight. The mixture is then cooled, and 10 ml of water are added. The mixture is extracted three times with in each case 10 ml of methylene chloride. The organic phases are dried over magnesium sulphate and the solvent is removed under reduced pressure. The residue is washed with water. This gives 251 mg (100% of theory) of the title compound.

¹H-NMR (300 MHz, DMSO-d₆): δ=3.24 (s, 2H), 5.36 (s, 2H), 7.06-7.13 (m, 2H), 7.26-7.33 (m, 2H), 8.85 (s, 1H).

MS (DCI): m/z=169 [M+H]⁺

HPLC (method 1): Rₜ=2.71 min

### Example 17A

### tert-Butyl 2-[(4-{[{[3-(4-fluorobenzyl)-1,2,4-oxadiazol-5-yl]methyl}(2-furylmethyl)amino]-methyl}phenyl)thio]-2-methylpropionate

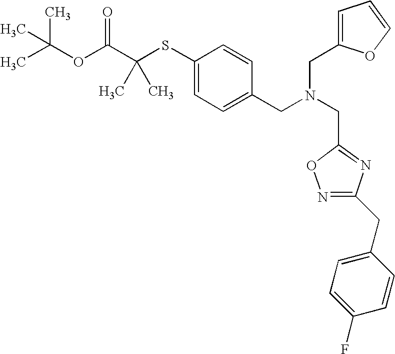

At −10° C., 77 mg of 1-hydroxy-1H-benzotriazole (0.57 mmol) and 72 mg of N,N-diisopropyl-carbodiimide (0.57 mmol) are added to 200 mg of N-{4-[(2-tert-butoxy-1,1-dimethyl-2-oxoethyl)thio]benzyl}-N-(2-furylmethyl)glycine (Example 3A) (0.477 mmol) and 96.2 mg of the compound from Example 16A (0.572 mmol) in 5 ml of dichloromethane/dimethylformamide (9:1). The mixture is stirred at −10° C. for 20 min and then at room temperature overnight. 15 ml of ethyl acetate are added to the reaction mixture. The mixture is then washed twice with saturated sodium bicarbonate solution, once with water, twice with 0.5 M potassium hydrogensulphate solution and once with saturated sodium chloride solution (in each case 10 ml). The organic phases are dried over magnesium sulphate and the solvent is removed under reduced pressure. The residue is taken up in 5 ml of ethanol. 43 mg of sodium acetate (0.52 mmol) and 20 μl of water are added, and the mixture is then heated under reflux overnight. 10 ml of water are added and the mixture is extracted three times with in each case 10 ml of ethyl acetate. The organic phases are dried over magnesium sulphate and the solvent is removed under reduced pressure. The residue is purified by preparative HPLC (mobile phase: acetonitrile/water with 0.1% formic acid, gradient 20:80→95:5). This gives 154 mg (59% of theory) of the title compound.

¹H-NMR (400 MHz, DMSO-d₆): δ=1.32 (s, 9H), 1.36 (s, 6H), 3.70 (s, 4H), 3.90 (s, 2H), 4.09 (s, 2H), 6.26-6.29 (m, 1H), 6.37-6.39 (m, 1H), 7.13-7.19 (m, 2H), 7.31-7.37 (m, 4H), 7.39-7.43 (m, 2H), 7.58-7.60 (m, 1H).

MS (ESIpos): m/z=552 [M+H]⁺

HPLC (method 1): Rₜ=5.67 min

### Example 18A

### tert-Butyl 2-({4-[((2-furylmethyl){[5-(4-methoxyphenyl)-1,2,4-oxadiazol-3-yl]methyl}amino)-methyl]phenyl}thio)-2-methylpropionate

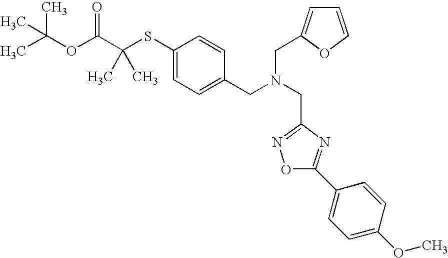

67 mg of 3-(chloromethyl)-5-(4-methoxyphenyl)-1,2,4-oxadiazole (0.30 mmol) and 0.11 ml of N,N-diisopropylethylamine (81 mg, 0.63 mmol) are added to 100 mg of tert-butyl 2-[(4-{[(2-furylmethyl)amino]methyl}phenyl)thio]-2-methylpropionate hydrochloride (0.251 mmol) [WO02/28821, Example II-3] in 2 ml of dimethylformamide. The mixture is stirred at 60° C. overnight. The reaction mixture is then purified directly by preparative HPLC (mobile phase: acetonitrile/water with 0.1% formic acid, gradient 20:80→95:5). This gives 38 mg (26% of theory) of the title compound.

¹H-NMR (400 MHz, DMSO-d₆): δ=1.33 (s, 9H), 1.36 (s, 6H), 3.75 (s, 2H), 3.77 (s, 2H), 3.81 (s, 2H), 3.88 (s, 3H), 6.36-6.38 (m, 1H), 6.42-6.44 (m, 1H), 7.18 (d, 2H), 7.42 (m, 4H), 7.62-7.64 (m, 1H), 8.07 (d, 2H).

MS (ESIpos): m/z=550 [M+H]⁺

HPLC (method 1): Rₜ=5.22 min

### Example 19A

### 3-(Chloromethyl)-1-phenyl-1H-1,2,4-triazole

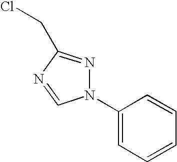

50 μl of thionyl chloride (82 mg, 0.68 mmol) are added to 100 mg of (1-phenyl-1H-1,2,4-triazol-3-yl)methanol (0.571 mmol) [preparation, for example, according to Huisgen et al., Chem. Ber. 98, 2185-2191 (1965)] in 2 ml of toluene. The mixture is stirred at 100° C. for 1 h and then concentrated under reduced pressure. 5 ml of toluene are added, and the mixture is again concentrated under reduced pressure. This step is repeated once more. The residue (101 mg) is reacted further without further purification.

### Example 20A

### (4-Phenyl-1H-imidazol-2-yl)methanol

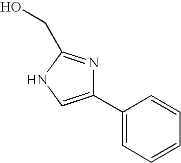

At 0° C., 1.37 ml of a 1 M lithium aluminium hydride solution in tetrahydrofuran are added to 297 mg of ethyl 4-phenyl-1H-imidazole-2-carboxylate (1.37 mmol) [preparation, for example, according to Song et al., J. Org. Chem. 64 (6), 1859-1867 (1999)] in 6 ml of tetrahydrofuran. The mixture is then stirred at room temperature overnight. 10 ml of water are then added, and the mixture is subsequently extracted three times with in each case 10 ml of ethyl acetate. The organic phases are dried over magnesium sulphate and concentrated, and the residue is washed with diethyl ether. This gives 176 mg (98% of theory) of the title compound.

MS (ESIpos): m/z=175 [M+H]⁺

HPLC (method 7): Rₜ=3.05 min

### Example 21A

### 2-(Chloromethyl)-4-phenyl-1H-imidazole

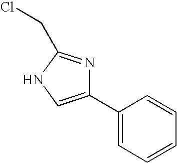

40 μl of thionyl chloride (66 mg, 0.55 mmol) are added to 80 mg of the compound from Example 20A (0.46 mmol) in 2 ml of toluene. The mixture is stirred at 100° C. for 1 h. The mixture is concentrated under reduced pressure. 5 ml of toluene are added, and the mixture is again concentrated under reduced pressure. This step is repeated once more. This gives a residue (80 mg) which is reacted further without further purification.

### Example 22A

### tert-Butyl 2-(4-cyanophenylsulphanyl)-2-methylpropionate

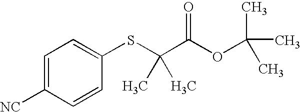

In a 26-litre tank, 2473 g (19.01 mol) of sodium sulphide (contains water) are suspended in 14.4 litres of NMP. At 125-130° C. and 110 mbar, 5.1 litres of the solvent are then removed again by distillation. At an internal temperature of 130-140° C., a solution of 2110 g (15.33 mol) of 4-chlorobenzonitrile in 3.84 litres of NMP is then added dropwise over a period of 1 hour. The temperature is increased to 155-160° C., and stirring is continued for 6 h. At 40-45° C., 3761 g (16.86 mol) of tert-butyl bromoisobutyrate are added over a period of 45 min. At 97° C. and 24 mbar, 13.0 litres of the solvent are then distilled off, the mixture is cooled to 90° C. and 5.8 litres of methylcyclohexane are added. The mixture is cooled to 15-20° C., 7.70 litres of water and 288 g of kieselguhr and are added and the mixture is stirred at 20° C. for 15 min. The mixture is then filtered through a porcelain Nutsche with a Seitz filter plate (K800), the filtrate is transferred into a 40-litre separating funnel and the phases are separated. The organic phase (9.1 litres) is twice stirred with in each case 5.8 litres of water, and the organic phase is concentrated on a rotary evaporator at 55-60° C./1 mbar. The residue obtained are 3788 g (89% of theory) of an oil which solidifies when stored at room temperature (purity 93% according to GC). The residue is used for the next step without further purification.

¹H-NMR (500 MHz, DMSO-d₆): δ=1.37 (s, 9H), 1.45 (s, 6H), 7.60 (d, 2H), 7.85 (d, 2H).

GC (method 8): Rₜ=17.2 min

### Example 23A

### tert-Butyl 2-[4-(aminomethyl)phenylsulphanyl]-2-methylpropionate hydrochloride

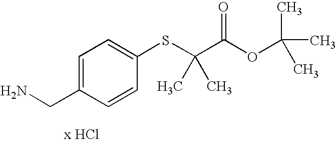

In a 26-litre tank, at 72° C., a solution of 2627 g (16.11 mol) of borane/N,N-diethylaniline complex is added dropwise over a period of 2 h to a solution of 3000 g (10.74 mol) of tert-butyl 2-(4-cyanophenylsulphanyl)-2-methylpropionate (Example 22A) in 5.5 litres of THF. The mixture is stirred at 72° C. for 1 h and then cooled to RT, and 2.33 litres of methanol are added over a period of 1 h. 5.81 litres of 6 M hydrochloric acid are then added, and the mixture is stirred at RT overnight. The mixture is transferred into a 40-litre separating funnel and the tank is rinsed with 3.88 litres of water and 7.75 litres of methylcyclohexane. The organic phase is stirred twice with in each case 3.8 litres of water. The combined aqueous phases are extracted with 3.88 litres of methylcyclohexane and then adjusted to pH 10.5 using concentrated aqueous sodium hydroxide solution (consumption: 2.5 litres). The aqueous/oily phase is stirred twice with in each case 3.88 litres of methylcyclohexane, and the combined organic phases are washed with 5.81 litres of water. The organic phase (14.5 litres) is concentrated on a rotary evaporator at 75° C./45 mbar. This gives 4.45 kg of a crude solution which comprises the desired product in a mixture with diethylaniline.

This crude solution is combined with an earlier batch of the same size, and most of the diethylaniline is distilled off in two steps via a thin-layer evaporator (1st distillation: product feed 458 g/h, feed temperature 80-85° C., pressure 2.7 mbar, head temperature 67° C., bottom temperature 37° C.; 2nd distillation: identical conditions at 1.0 mbar). The distillation residue (3664 g) is charged to an enamel tank in 7.8 litres of MTBE, and a 5- to 6-molar solution of hydrogen chloride in isopropanol is added dropwise over a period of 20 min. During the addition, the internal temperature rises to 47° C. The suspension is cooled to RT and stirred for another 2 h. The mixture is filtered off with suction through a Seitz filter plate, and the filter plate is washed four times with in each case 2.6 litres of MTBE. The moist product (5.33 kg) is dried under reduced pressure at 40° C. and a nitrogen blanket until the mass remains constant. The two combined batches yield 2780 g (41% of theory) of the title compound as white crystals.

¹H-NMR (400 MHz, DMSO-d₆): δ=1.39 (m, 15H), 4.04 (s, 2H), 7.49 (m, 4H), 8.48 (br. s, 3H).

MS (DCI/NH₃): m/z=282 [M+H]⁺, 299 [M+NH₄]⁺.

### Example 24A

### Methyl (2Z)-3-oxo-2-(phenyliodanylidene)butanoate

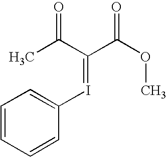

At −5° C., a solution of 39.20 g (698.63 mmol) of potassium hydroxide in 250 ml of methanol is added dropwise to a solution of 18.31 g (157.68 mmol) of methyl acetoacetate in 100 ml of methanol. A solution of 50.80 g (157.68 mmol) of iodobenzene diacetate in 250 ml of methanol is then added dropwise. After two hours of stirring at 0° C., the mixture is poured into 500 ml of ice-water and the precipitate is filtered off with suction and washed with a little water. Drying gives 32.90 g (65% of theory) of the title compound in the form of colourless crystals.

LC/MS (method 3): Rₜ=2.56 min

¹H-NMR (400 MHz, DMSO-d₆): δ [ppm]=2.39 (s, 3H), 3.52 (s, 3H), 7.40-7.44 (m, 2H), 7.48-7.52 (m, 1H), 7.70-7.75 (m, 2H).

### Example 25A

### Methyl 5-methyl-2-(3-methylbenzyl)-1,3-oxazole-4-carboxylate

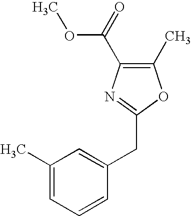

With vigorous stirring, a suspension of 10.00 g (31.44 mmol) of the compound from Example 24A, 20.60 g (157.18 mmol) of m-tolylacetonitrile and 0.22 g (0.50 mmol) of rhodium diacetate dimer is immersed into an oil bath at a temperature of 100° C. for 15 minutes. After cooling to room temperature, the mixture is filtered through silica gel (mobile phase: isohexane/ethyl acetate 50:50) and then purified by preparative HPLC (mobile phase: acetonitrile/water with 0.1% formic acid, gradient 20:80→95:5). This gives 3.10 g (41% of theory) of the title compound in the form of a dark yellow oil.

LC/MS (method 2): Rₜ=2.41 min; MS (ESIpos): m/z=246 [M+H]⁺

¹H-NMR (400 MHz, CDCl₃): δ [ppm]=2.33 (s, 3H), 2.56 (s, 3H), 3.90 (s, 3H), 4.05 (s, 2H), 7.06-7.10 (m, 3H), 7.19-7.23 (m, 1H).

The following compound is prepared analogously to Example 25A from the starting materials stated:

| | | | Yield | |
|---|---|---|---|---|
| | | Starting | [% of | |
| Example | Structure | material | theory] | LC/MS |
| 26A | 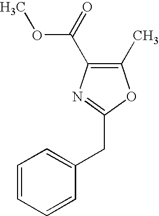 | benzylnitrile | 56% | Rₜ = 2.16 min; MS(ESIpos): m/z = 232[M + H]⁺(method 5) |

### Example 27A

### 2-(3-Methylphenyl)acetamide

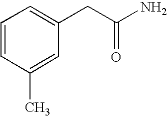

With ice-cooling, 29.94 g (177.93 mmol) of a 25% strength aqueous ammonia solution are added dropwise to a solution of 6.00 g (35.38 mmol) of (3-methylphenyl)acetyl chloride in 100 ml of dioxane. After 15 minutes at room temperature, 200 ml of ice-water are added to the reaction mixture, and the pH is then adjusted to 2 using concentrated hydrochloric acid. Most of the dioxane is removed, and the precipitated solid is filtered off, washed with water and n-pentane and dried at 60° C. in a vacuum drying cabinet. This gives 4.97 g (94% of theory) of the title compound in the form of colourless crystals.

LC/MS (method 3): Rₜ=1.41 min; MS (ESIpos): m/z=150 [M+H]⁺

¹H-NMR (400 MHz, DMSO-d₆): δ [ppm]=2.28 (s, 3H), 3.32 (s, 2H), 6.83 (s, 1H, NH), 7.04 (m, 3H), 7.17 (m, 1H), 7.43 (s, 1H, NH).

The following compounds are prepared analogously to Example 27A from the starting materials stated in each case:

| | | | Yield | |
|---|---|---|---|---|
| | | | [% of | |
| Example | Structure | Starting material | theory] | LCMS |
| 28A | 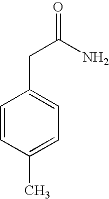 | (4-methylphenyl)-acetyl chloride | 92% | Rₜ = 1.42 min; MS(ESIpos): m/z = 150[M + H]⁺(method 3) |
| 29A | 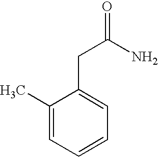 | (2-methylphenyl)-acetyl chloride | 99% | Rₜ = 1.33 min; MS(ESIpos): m/z = 150[M + H]⁺(method 3) |
| 30A | 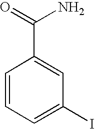 | 3-iodobenzoylchloride | 98% | Rₜ = 1.37 min; MS(ESIpos): m/z = 248[M + H]⁺(method 2) |

### Example 31A

### Ethyl 4-methyl-2-(3-methylbenzyl)-1,3-oxazole-5-carboxylate

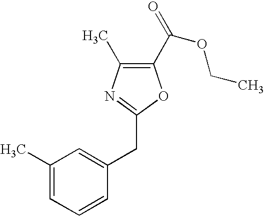

A suspension of 3.29 g (20.00 mmol) of ethyl 2-chloroacetoacetate and 3.88 g (26.00 mmol) of the compound from Example 27A is heated at 150° C. for 2 hours. After cooling, the crude product is filtered through silica gel (mobile phase: dichloromethane) and then purified over a Biotage cartridge 40M (mobile phase: isohexane/ethyl acetate 90:10). Removal of the solvent gives 2.71 g (52% of theory) of the title compound in the form of a yellowish oil.

LC/MS (method 3): Rₜ=2.53 min; MS (ESIpos): m/z=260 [M+H]⁺

¹H-NMR (400 MHz, DMSO-d₆): δ [ppm]=1.37 (t, 3H), 2.33 (s, 3H), 2.44 (s, 3H), 4.07 (s, 2H), 4.35 (q, 2H), 7.08 (m, 3H), 7.23 (m, 1H).

The following compounds are prepared analogously to Example 31A from the starting materials given in each case:

| | | | Yield | |
|---|---|---|---|---|
| | | | [% of | |
| Example | Structure | Starting materials | theory] | LC/MS |
| 32A | 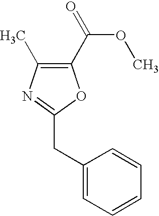 | 2-phenylacetamide;methyl-2-chloroaceto-acetate | 39% | Rₜ = 1.96 min; MS(ESIpos): m/z = 232[M + H]⁺(method 2) |
| 33A | 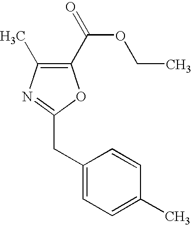 | Example 28A;ethyl 2-chloroaceto-acetate | 50% | Rₜ = 2.55 min; MS(ESIpos): m/z = 260[M + H]⁺(method 5) |
| 34A | 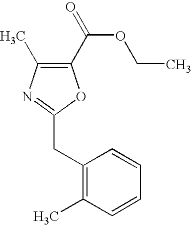 | Example 29A;ethyl 2-chloroaceto-acetate | 46% | Rₜ = 2.50 min; MS(ESIpos): m/z = 260[M + H]⁺(method 3) |
| 35A | 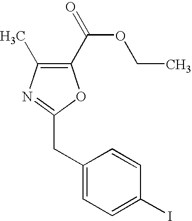 | Example 30A;ethyl 2-chloroaceto-acetate | 11% | Rₜ = 2.75 min; MS(ESIpos): m/z = 358[M + H]⁺(method 2) |

### Example 36A

### Ethyl 4-methyl-2-(3-pyridin-3-ylphenyl)-1,3-oxazole-5-carboxylate

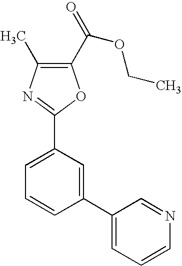

At 80° C., argon is passed through a solution of 0.20 g (0.56 mmol) of the compound from Example 35A and 0.21 g (1.68 mmol) of 3-pyridylboronic acid in 6 ml of DMF and 0.62 ml (1.23 mmol) of 2N sodium carbonate solution. After five minutes, 0.04 g (0.06 mmol) of [1,1′-bis(diphenyl-phosphino)ferrocene]palladium(II) chloride/dichloromethane complex is added, and the mixture is stirred at this temperature for 1 hour. The mixture is then cooled to room temperature, taken up in ethyl acetate and water and filtered through Celite. The aqueous phase is extracted with ethyl acetate, and the combined organic phases are washed three times with water and then with saturated sodium chloride solution and dried over anhydrous magnesium sulphate. The crude product which remains after removal of the solvent is purified over a Biotage cartridge 40S (mobile phase: isohexane/ethyl acetate 1:9). This gives 0.31 g (97% of theory) of the title compound in the form of colourless crystals.

LC/MS (method 5): Rₜ=2.35 min; MS (ESIpos): m/z=309 [M+H]⁺

¹H-NMR (400 MHz, CDCl₃): δ [ppm]=1.43 (t, 3H), 2.57 (s, 3H), 4.43 (q, 2H), 7.42 (q, 1H), 7.61 (t, 1H), 7.73 (m, 1H), 7.97 (m, 1H), 8.17 (m, 1H), 8.34 (m, 1H), 8.64 (m, 1H), 8.92 (s, 1H).

### Example 37A

### 2-(3-Methylphenyl)ethanethioamide

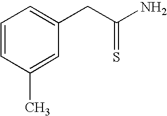

A solution of 18.00 g (120.65 mmol) of the compound from Example 27A and 29.28 g (72.39 mmol) of Lawesson's reagent in 500 ml of anhydrous THF is heated under reflux for 90 min. The solvent is then removed, and the residue is purified on 600 g of silica gel 60 by flash chromatography (mobile phase: cyclohexane/ethyl acetate 4:1). The product fractions are checked by TLC and concentrated, and the residue is triturated with n-heptane. The precipitate is filtered off with suction and washed with n-heptane. This gives 16.16 g (81% of theory) of the title compound in the form of colourless crystals.

LC/MS (method 2): Rₜ=1.57 min; MS (ESIpos): m/z=166 [M+H]⁺

¹H-NMR (400 MHz, CDCl₃): δ [ppm]=2.36 (s, 3H), 4.08 (s, 2H), 6.69 (s, 1H, NH), 7.04-7.15 (m, 3H), 7.25-7.3 (m, 1H), 7.68 (s, 1H, NH).

### Example 38A

### Ethyl 3-bromo-2-oxobutanoate

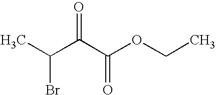

6.35 g (50.18 mmol) of ethyl 2-oxobutanoate are initially charged in 500 ml of ethyl acetate, and a solution of 33.62 g (150.53 mmol) of copper(II) bromide in 250 ml of chloroform is added. The mixture is heated under reflux for five hours and, after cooling, purified over 200 g of silica gel 60 (mobile phase: cyclohexane/ethyl acetate 3:1). This gives 8.22 g (78% of theory) of the title compound in the form of a yellow oil.

¹H-NMR (400 MHz, CDCl₃): δ [ppm]=1.40 (t, 3H), 1.81 (d, 3H), 4.38 (m, 2H), 5.17 (q, 1H).

### Example 39A

### Ethyl 4-methyl-2-(3-methylbenzyl)-1,3-thiazole-5-carboxylate

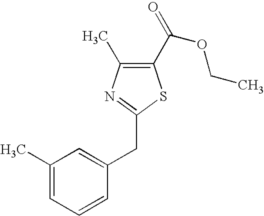

A suspension of 15.45 g (93.50 mmol) of the compound from Example 37A in 16.8 ml (121.53 mmol) of ethyl 2-chloroacetoacetate is stirred in an oil bath at 150° C. for 45 minutes. After cooling, the mixture is taken up in dichloromethane and purified over 400 g of silica gel 60 by flash chromatography (mobile phase: cyclohexane/ethyl acetate 75:25). The crude product obtained is purified further by preparative HPLC column (column: 230 mm×50 mm, silica gel Si 60, 12 μm, from Merck; mobile phase: isohexane/ethyl acetate 90:10). This gives 13.71 g (53% of theory) of the title compound in the form of a brown oil.

LC/MS (method 2): Rₜ=2.59 min; MS (ESIpos): m/z=276 [M+H]⁺

¹H-NMR (400 MHz, CDCl₃): δ [ppm]=1.31 (t, 3H), 2.35 (s, 3H), 2.71 (s, 3H), 4.22 (s, 2H), 4.28 (q, 2H), 7.09-7.12 (m, 3H), 7.24 (m, 1H).

The following compound is prepared analogously to Example 39A from the starting materials stated:

| | | | Yield | |
|---|---|---|---|---|
| | | Starting | [% of | |
| Example | Structure | material | theory] | LC/MS |
| 40A | 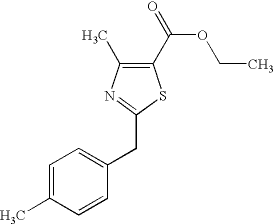 | 2-(4-methylphenyl)ethane-thioamide | 72% | Rₜ = 2.62 min; MS(ESIpos): m/z = 276[M + H]⁺(method 2) |

### Example 41A

### Ethyl 5-methyl-2-(3-methylbenzyl)-1,3-thiazole-4-carboxylate

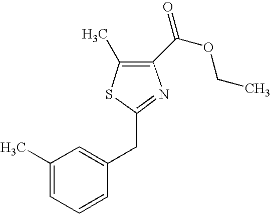

A mixture of 1.00 g (4.78 mmol) of the compound from Example 37A and 0.95 g (5.74 mmol) of the compound from Example 38A is stirred at 120° C. for about 30 minutes. After cooling, the mixture is taken up in ethyl acetate and insoluble components are filtered off through Celite. The crude product obtained after concentration is purified over a Biotage 40M cartridge (mobile phase: isohexane/ethyl acetate 90:10). This gives 0.32 g (24% of theory) of the title compound as a yellow oil.

LC/MS (method 3): Rₜ=2.63 min; MS (ESIpos): m/z=276 [M+H]⁺

¹H-NMR (400 MHz, CDCl₃): δ [ppm]=1.42 (t, 3H), 2.34 (s, 3H), 2.68 (s, 3H), 4.26 (s, 2H), 4.43 (q, 2H), 7.08-7.12 (m, 3H), 7.21 (m, 1H).

### Example 42A

### Ethyl (2E)-chloro[(3-methylphenyl)hydrazono]acetate

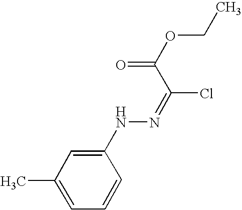

7.08 ml (50.81 mmol) of ethyl 2-chloroacetoacetate are added to a solution of 6.92 g (50.81 mmol) of sodium acetate trihydrate in a mixture of 300 ml of ethanol and 15 ml of water. After 15 minutes, the solution is cooled to an internal temperature of 0° C. In parallel to this reaction, 50 ml of a solution of 3.51 g (50.81 mmol) of sodium nitrite in 65 ml of water are added dropwise to a suspension, cooled to about 0° C., of 5.45 g (50.81 mmol) of m-toluidine in 80 ml of 6 M hydrochloric acid. After the dropwise addition has ended, stirring at 0° C. is continued for about 10 minutes and the diazonium salt solution formed is then added dropwise to the first solution, the temperature not exceeding 0° C. Stirring at 0° C. is continued for 1 h, and about half of the solvent is then removed on a rotary evaporator. The residue is stored at −26° C. overnight. The precipitated solid is filtered off with suction and dried under reduced pressure. This gives 4.86 g (38% of theory) of the title compound in the form of reddish-brown crystals.

LC/MS (method 5): Rₜ=2.62 min; MS (ESIpos): m/z=241 [M+H]⁺

¹H-NMR (400 MHz, CDCl₃): δ [ppm]=1.41 (t, 3H), 2.36 (s, 3H), 4.39 (q, 2H), 6.86 (d, 1H), 7.01 (d, 1H), 7.06 (s, 1H), 7.21 (m, 1H), 8.30 (br. s, 1H).

The following compounds are prepared analogously to Example 42A from the starting materials stated in each case:

| | | | Yield | |
|---|---|---|---|---|
| | | Starting | [% of | |
| Example | Structure | material | theory] | LC/MS |
| 43A | 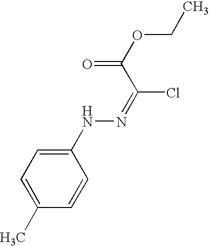 | p-Toluidine | 49% | Rₜ = 2.62 min; MS (ESIpos):m/z = 241 [M + H]⁺ (method 5) |
| 44A | 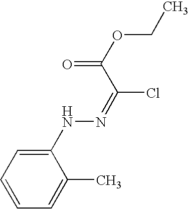 | o-Toluidine | 23% | Rₜ = 2.46 min; MS (ESIpos):m/z = 241 [M + H]⁺ (method 2) |

### Example 45A

### Ethyl [(2-fluorobenzyl)hydrazono]acetate

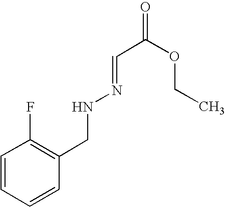

Under reflux, a solution of 31.8 g (168.3 mmol) of 2-fluorobenzyl bromide in 150 ml of ethanol is added dropwise over a period of 1 h to a solution of 38.30 g (758 mmol) of hydrazine hydrate in 200 ml of ethanol. The mixture is stirred at reflux temperature for 5 h and then at RT overnight. The solvent is distilled off and the residue is taken up in water and then extracted twice with diethyl ether. The organic phases are combined and dried with sodium sulphate, and the solvent is removed by distillation under reduced pressure. This gives 23.5 g (99.6% of theory) of (2-fluorobenzyl)hydrazine which is converted into the hydrochloride by precipitation with hydrogen chloride in diethyl ether. The hydrochloride is used without further purification steps.

13.36 g (76 mmol) of (2-fluorobenzyl)hydrazine hydrochloride and 9.31 g (113 mmol) of sodium acetate are dissolved in 100 ml of ethanol. 15.0 ml (76 mmol) of ethyl glyoxylate (50% strength in toluene) are then added, and the mixture is stirred at RT overnight. The solvent is distilled off and the residue is taken up in dichloromethane and washed successively with water, 50% strength ammonium chloride solution and 50% strength potassium carbonate solution. After drying over sodium sulphate, the solvent is removed on a rotary evaporator and the residue is purified by flash chromatography (silica gel, mobile phase: dichloromethane→dichloromethane/ethyl acetate 10:1). This gives 9.52 g (56% of theory, based on the (2-fluorobenzyl)hydrazine hydrochloride) of the title compound.

LC/MS (method 5): Rₜ=2.00 min; MS (ESIpos): m/z=225 [M+H]⁺

¹H-NMR (300 MHz, CDCl₃): δ [ppm]=1.32 (t, 3H), 4.27 (q, 2H), 4.49 (d, 2H), 6.63 (br. s, 1H), 6.81 (s, 1H), 7.02-7.20 (m, 2H), 7.24-7.36 (m, 2H).

### Example 46A

### Ethyl 2-chloro[(2-fluorobenzyl)hydrazono]acetate

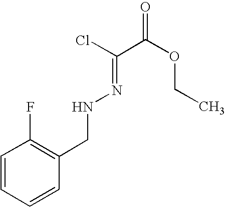

A solution of 8.50 g (37.9 mmol) of the compound from Example 45A and 4.15 g (31.1 mmol) of N-chlorosuccinimide in 100 ml of ethanol is stirred at 60° C. for one hour. After the reaction has ended (checked by TLC), the reaction mixture is concentrated, the residue is taken up in chloroform, the solid that remains is filtered off, the solvent is removed under reduced pressure and the residue is then purified by flash chromatography (silica gel, mobile phase: dichloromethane). This gives 5.88 g (60% of theory) of the title compound.

LC/MS (method 2): Rₜ=2.18 min; MS (ESIpos): m/z=259 [M+H]⁺

¹H-NMR (300 MHz, DMSO-d₆): δ [ppm]=1.22 (t, 3H), 4.20 (q, 2H), 4.60 (d, 2H), 7.13-7.24 (m, 2H), 7.27-7.38 (m, 2H), 8.71 (t, 1H).

### Example 47A

### Ethyl 1-(2-fluorobenzyl)-4-methyl-1H-pyrazole-3-carboxylate

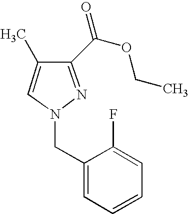

A suspension of 12.10 g (36.78 mmol) of the compound from Example 46A, 27.10 g (116.94 mmol) of silver(I) oxide and 13.08 ml (116.94 mmol) of ethyl propenyl ether in 210 ml of anhydrous dioxane is heated under reflux for 3.5 hours. The mixture is filtered through Celite, the filter cake is washed with ethyl acetate and the filtrate is concentrated under reduced pressure. The residue is taken up in ethyl acetate, washed with water and with saturated sodium chloride solution and dried over anhydrous magnesium sulphate. The crude product is purified by preparative HPLC (mobile phase: acetonitrile/water with 0.1% formic acid, gradient 20:80→95:5). This gives 4.29 g (21% of theory) of the title compound in the form of a red-brown oil.

LC/MS (method 5): Rₜ=2.35 min; MS (ESIpos): m/z=263 [M+H]⁺

¹H-NMR (400 MHz, CDCl₃): δ [ppm]=1.40 (t, 3H), 2.26 (s, 3H), 4.40 (q, 2H), 5.39 (s, 2H), 7.06-7.36 (m, 5H).

The following compounds are prepared analogous to Example 47A from the starting materials stated in each case:

| | | | Yield | |
|---|---|---|---|---|
| | | Starting | [% of | |
| Example | Structure | material | theory] | LC/MS |
| 48A | 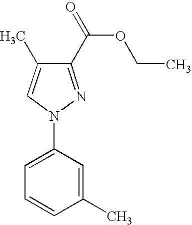 | Example 42A | 50% | Rₜ = 2.38 min; MS (ESIpos):m/z = 245 [M + H]⁺ (method 2) |
| 49A | 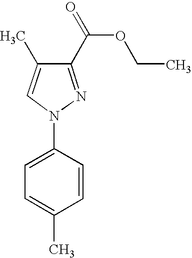 | Example 43A | 54% | Rₜ = 2.37 min; MS (ESIpos):m/z = 245 [M + H]⁺ (method 2) |
| 50A | 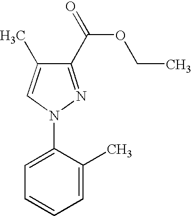 | Example 44A | 38% | Rₜ = 2.45 min; MS (ESIpos):m/z = 245 [M + H]⁺ (method 3) |

### Example 51A

### [5-Methyl-2-(3-methylbenzyl)-1,3-oxazol-4-yl]methanol

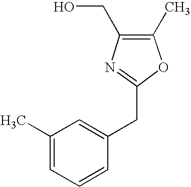

A solution of 5.00 g (20.39 mmol) of the compound from Example 25A in 100 ml of anhydrous THF is cooled to 0° C., and 12.3 ml (12.23 mmol) of a 1 M lithium aluminium hydride solution in THF are added dropwise. After 20 min, the cooling bath is removed and the mixture is stirred at room temperature for one hour. The mixture is then again cooled to 0° C., and ethanol is added carefully until the evolution of gas has ceased. 50 ml of a saturated potassium sodium tartrate solution are then added. The mixture is stirred at room temperature for 12 h, the phases are then separated and the aqueous phase is extracted twice with ethyl acetate. The combined organic phases are washed with saturated sodium chloride solution and dried over anhydrous magnesium sulphate. Removal of the solvent gives a yellow oil which, after flash chromatography (silica gel, mobile phase: isohexane/ethyl acetate 50:50), yields 2.94 g (66% of theory) of the title compound in the form of colourless crystals.

LC/MS (method 2): Rₜ=1.68 min; MS (ESIpos): m/z=218 [M+H]⁺

¹H-NMR (400 MHz, CDCl₃): δ [ppm]=2.26 (s, 3H), 2.33 (s, 3H), 3.99 (s, 2H), 4.48 (s, 2H), 7.05-7.10 (m, 3H), 7.18-7.22 (m, 1H).

The following compounds are prepared analogously to Example 51A from the starting materials stated:

| | | | Yield | |
|---|---|---|---|---|
| | | Starting | [% of | |
| Example | Structure | material | theory] | LC/MS |
| 52A | 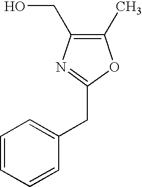 | Example 26A | 36% | Rₜ = 1.75 min; MS (ESIpos):m/z = 204 [M + H]⁺ (method 5) |
| 53A | 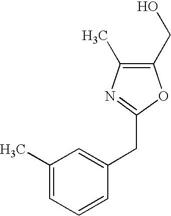 | Example 31A | 70% | Rₜ = 1.56 min; MS (ESIpos):m/z = 218 [M + H]⁺ (method 2) |
| 54A | 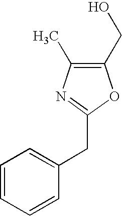 | Example 32A | 43% | Rₜ = 1.34 min; MS (ESIpos):m/z = 204 [M + H]⁺ (method 2) |
| 55A | 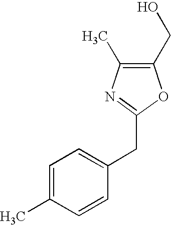 | Example 33A | 64% | Rₜ = 1.83 min; MS (ESIpos):m/z = 218 [M + H]⁺ (method 5) |
| 56A | 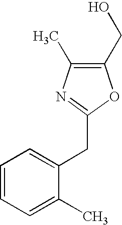 | Example 34A | 68% | Rₜ = 1.51 min; MS (ESIpos):m/z = 218 [M + H]⁺ (method 5) |
| 57A | 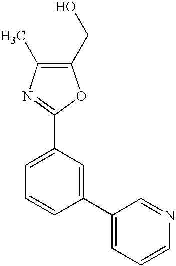 | Example 36A | 99% | Rₜ = 1.17 min; MS (ESIpos):m/z = 267 [M + H]⁺ (method 2) |
| 58A | 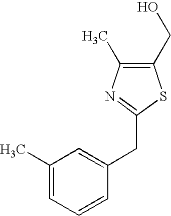 | Example 39A | 80% | Rₜ = 1.91 min; MS (ESIpos):m/z = 234 [M + H]⁺ (method 3) |
| 59A | 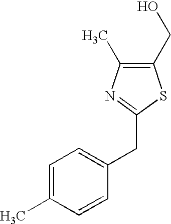 | Example 40A | 99% | Rₜ = 1.69 min; MS (ESIpos):m/z = 234 [M + H]⁺ (method 2) |
| 60A | 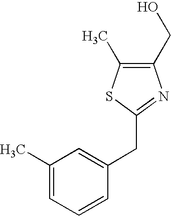 | Example 41A | 84% | Rₜ = 2.05 min; MS (ESIpos):m/z = 234 [M + H]⁺ (method 3) |
| 61A | 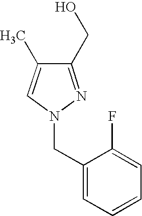 | Example 47A | 87% | Rₜ = 1.72 min; MS (ESIpos):m/z = 221 [M + H]⁺ (method 3) |
| 62A | 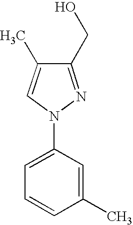 | Example 48A | 73% | Rₜ = 1.95 min; MS (ESIpos):m/z = 203 [M + H]⁺ (method 5) |
| 63A | 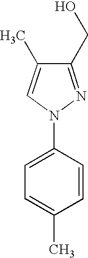 | Example 49A | 48% | Rₜ = 1.92 min; MS (ESIpos):m/z = 203 [M + H]⁺ (method 3) |
| 64A | 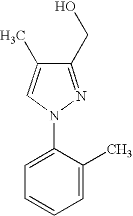 | Example 50A | 85% | Rₜ = 1.83 min; MS (ESIpos):m/z = 203 [M + H]⁺ (method 5) |

### Example 65A

### 4-(Chloromethyl)-5-methyl-2-(3-methylbenzyl)-1,3-oxazole

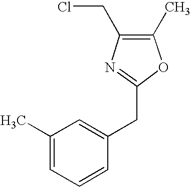

At 0° C., 1.32 g (6.93 mmol) of p-tolylsulphonyl chloride are added a little at a time to a solution of 1.25 g (5.77 mmol) of the compound from Example 51A and 0.92 g (7.50 mmol) of N,N-dimethylpyridine-4-amine in 10 ml of dry dichloromethane. After one hour of stirring at room temperature, the mixture is purified by flash chromatography (silica gel, mobile phase: isohexane/ethyl acetate 85:15). This gives 1.09 g (80% of theory) of the title compound in the form of a colourless oil.

MS (ESIpos): m/z=236 [M+H]⁺

¹H-NMR (400 MHz, CDCl₃): δ [ppm]=2.28 (s, 3H), 2.33 (s, 3H), 4.00 (s, 2H), 4.46 (s, 2H), 7.06-7.10 (m, 3H), 7.19-7.30 (m, 1H).

The following compounds are prepared analogously to Example 65A from the starting materials stated:

| | | | Yield | |
|---|---|---|---|---|
| | | Starting | [% of | |
| Example | Structure | material | theory] | MS (ESIpos) |
| 66A | 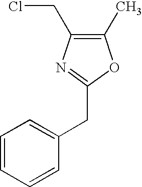 | Example 52A | 85% | m/z = 222 [M + H]⁺ |
| 67A | 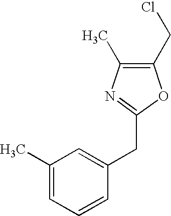 | Example 53A | 84% | m/z = 236 [M + H]⁺ |
| 68A | 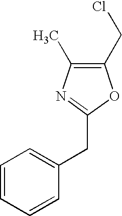 | Example 54A | 83% | m/z = 222 [M + H]⁺ |
| 69A | 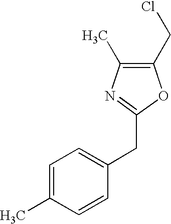 | Example 55A | 83% | m/z = 236 [M + H]⁺ |
| 70A | 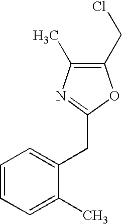 | Example 56A | 81% | m/z = 236 [M + H]⁺ |
| 71A | 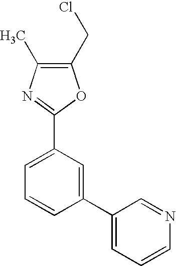 | Example 57A | 80% | m/z = 285 [M + H]⁺ |
| 72A | 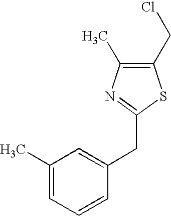 | Example 58A | 83% | m/z = 252 [M + H]⁺ |
| 73A | 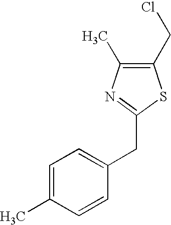 | Example 59A | 89% | m/z = 252 [M + H]⁺ |
| 74A | 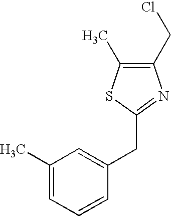 | Example 60A | 93% | m/z = 252 [M + H]⁺ |
| 75A | 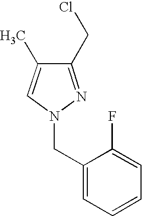 | Example 61A | 77% | m/z = 239 [M + H]⁺ |
| 76A | 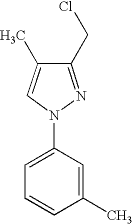 | Example 62A | 74% | m/z = 221 [M + H]⁺ |
| 77A | 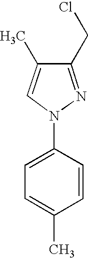 | Example 63A | 87% | m/z = 221 [M + H]⁺ |
| 78A | 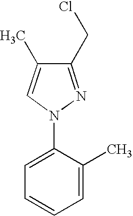 | Example 64A | 98% | m/z = 221 [M + H]⁺ |

### Example 79A

### tert-Butyl 2-({4-[((2-furylmethyl) {[5-methyl-2-(3-methylbenzyl)-1,3-oxazol-4-yl]methyl}amino)-methyl]phenyl}thio)-2-methylpropionate

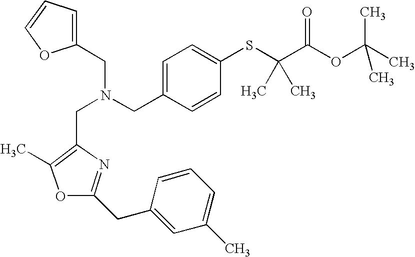

0.66 g (4.76 mmol) of potassium carbonate is added to a solution of 0.86 g (2.38 mmol) of the compound from Example 65A and 0.51 g (2.16 mmol) of tert-butyl 2-[(4-{[(2-furylmethyl)amino]methyl}phenyl)thio]-2-methylpropionate [WO 02/28821, Example II-3 (free base)] in 2 ml of anhydrous DMF, and the mixture is stirred at 90° C. for one hour. After cooling, ethyl acetate and water are added to the mixture. The aqueous phase is extracted once with ethyl acetate. The combined organic phases are washed four times with water and once with saturated sodium chloride solution and dried over anhydrous magnesium sulphate. The crude product obtained after removal of the solvent is purified over a Biotage cartridge 40M (mobile phase: dichloromethane/ethyl acetate 20:1). This gives 0.96 g (79% of theory) of the title compound in the form of a yellowish oil.

LC/MS (method 3): Rₜ=3.04 min; MS (ESIpos): m/z=561 [M+H]⁺

¹H-NMR (400 MHz, CDCl₃): δ [ppm]=1.41 (s, 9H), 1.42 (s, 6H), 2.16 (s, 3H), 2.32 (s, 3H), 3.49 (s, 2H), 3.60 (s, 2H), 3.67 (s, 2H), 4.00 (s, 2H), 6.19 (m, 1H), 6.32 (m, 1H), 7.04-7.11 (m, 3H), 7.19 (m, 1H), 7.32 (d, 2H), 7.38 (m, 1H), 7.43 (d, 2H).

The following compounds are prepared analogously to Example 79A from the starting materials stated:

| | | | Yield | |
|---|---|---|---|---|
| Exam- | | Starting | [% of | |
| ple | Structure | material | theory] | LC/MS |
| 80A | 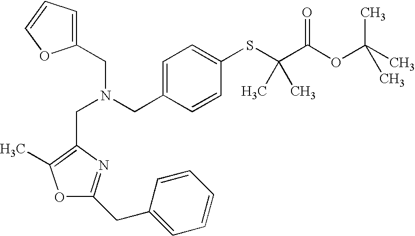 | Example 66A | 85% | Rₜ = 2.88 min; MS (ESIpos):m/z = 547 [M + H]⁺ (method 5) |
| 81A | 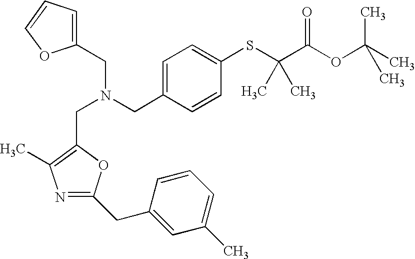 | Example 42A | 53% | Rₜ = 3.21 min; MS (ESIpos):m/z = 561 [M + H]⁺ (method 2) |
| 82A | 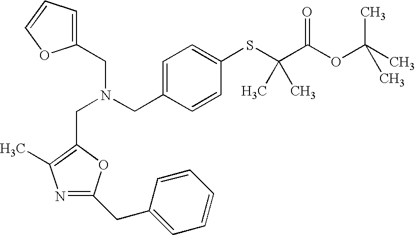 | Example 68A | 76% | Rₜ = 3.32 min; MS (ESIpos):m/z = 547 [M + H]⁺ (method 3) |
| 83A | 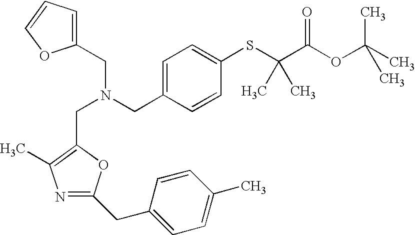 | Example 69A | 83% | Rₜ = 3.41 min; MS (ESIpos):m/z = 561 [M + H]⁺ (method 3) |
| 84A | 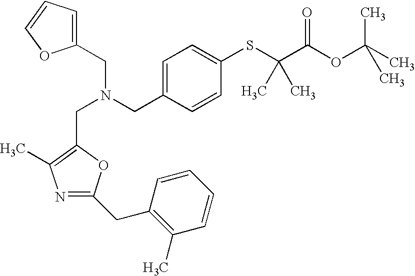 | Example 70A | 67% | Rₜ = 3.41 min; MS (ESIpos):m/z = 561 [M + H]⁺ (method 5) |
| 85A | 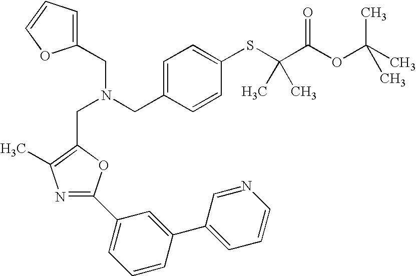 | Example 71A | 40% | Rₜ = 3.33 min; MS (ESIpos):m/z = 610 [M + H]⁺ (method 2) |
| 86A | 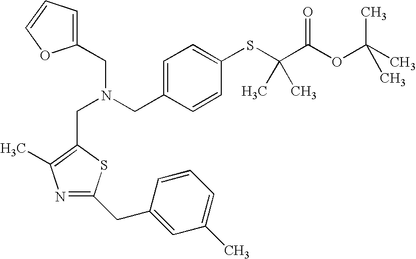 | Example 72A | 66% | Rₜ = 3.55 min; MS (ESIpos):m/z = 577 [M + H]⁺ (method 5) |
| 87A | 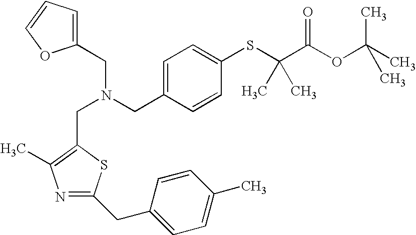 | Example 73A | 47% | Rₜ = 3.57 min; MS (ESIpos):m/z = 577 [M + H]⁺ (method 3) |
| 88A | 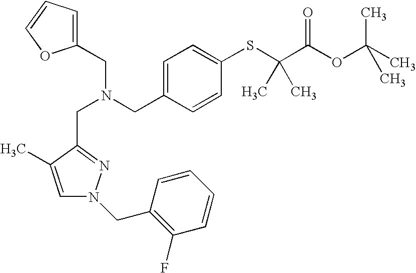 | Example 75A | 42% | Rₜ = 2.68 min; MS (ESIpos):m/z = 564 [M + H]⁺ (method 2) |
| 89A | 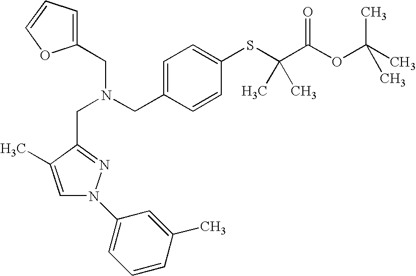 | Example 76A | 63% | Rₜ = 3.22 min; MS (ESIpos):m/z = 546 [M + H]⁺ (method 5) |
| 90A | 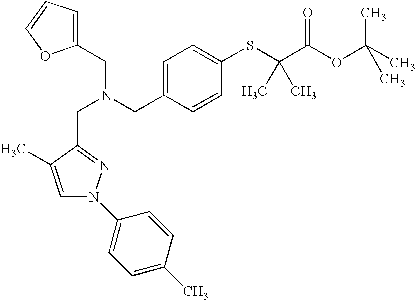 | Example 77A | 71% | Rₜ = 3.13 min; MS (ESIpos):m/z = 546 [M + H]⁺ (method 3) |
| 91A | 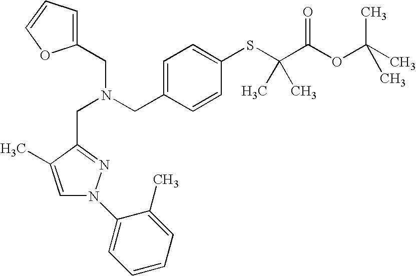 | Example 78A | 74% | Rₜ = 2.95 min; MS (ESIpos):m/z = 546 [M + H]⁺ (method 2) |

### Example 92A

### tert-Butyl 2-[(4-{[(2-methoxyethyl)amino]methyl}phenyl)thio]-2-methylpropionate

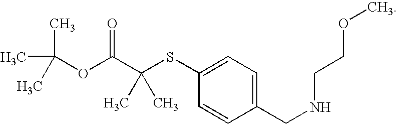

### CH₃₀OCH₃

5.00 g (15.73 mmol) of the compound from Example 23A are initially charged in 15 ml of DMF, and 1.97 g of 2-bromoethyl methyl ether (14.16 mmol) and 5.48 ml of triethylamine (39.32 mmol) are added at RT. The mixture is stirred at RT overnight and then concentrated on a rotary evaporator. Water is added to the residue, and the mixture is extracted two times with ethyl acetate. The combined organic phases are dried over sodium sulphate and the solvent is distilled off under reduced pressure. The residue is purified by flash chromatography on silica gel (mobile phase: dichloromethane/isopropanol 5:1). This gives 2.56 g (48% of theory) of the title compound.

LC/MS (method 2): Rₜ=1.49 min; MS (ESIpos): m/z=340 [M+H]⁺

¹H-NMR (400 MHz, DMSO-d₆): δ [ppm]=1.38 (s, 15H), 3.09 (t, 2H), 3.30 (s, 3H), 3.58 (t, 2H), 4.18 (s, 2H), 7.51 (s, 4H), 8.92 (br. s, 1H).

### Example 93A

### tert-Butyl 2-({4-[((2-methoxyethyl) {[5-methyl-2-(3-methylbenzyl)-1,3-oxazol-4-yl]methyl}-amino)methyl]phenyl}thio)-2-methylpropionate

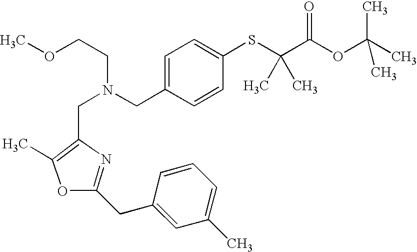

0.41 g (2.94 mmol) of potassium carbonate is added to a solution of 0.35 g (1.47 mmol) of the compound from Example 65A and 0.50 g (1.47 mmol) of the compound from Example 92A in 2 ml of anhydrous DMF, and the mixture is stirred at 90° C. for one hour. After cooling, ethyl acetate and water are added to the mixture. The aqueous phase is reextracted once with ethyl acetate. The combined organic phases are washed four times with water and once with saturated sodium chloride solution and dried over anhydrous magnesium sulphate. The crude product obtained after removal of the solvent is purified over a Biotage cartridge 40M (mobile phase: isohexane/ethyl acetate 75:25). This gives 0.48 g (60% of theory) of the title compound in the form of a colourless oil.

LC/MS (method 9): Rₜ=2.14 min; MS (ESIpos): m/z=539 [M+H]⁺

¹H-NMR (400 MHz, CDCl₃): δ=1.41 (s, 9H), 1.42 (s, 6H), 2.17 (s, 3H), 2.32 (s, 3H), 2.71 (t, 2H, J=5.8 Hz), 3.29 (s, 3H), 3.48 (t, 2H, J=5.8 Hz), 3.52 (s, 2H), 3.66 (s, 2H), 3.99 (s, 2H), 7.04-7.09 (m, 3H), 7.19 (m, 1H), 7.30 (d, 2H, J=8.9 Hz), 7.42 (2H, d, J=8.9 Hz).

The following compounds are prepared analogously to Example 93A from the starting materials stated:

| | | | Yield | |
|---|---|---|---|---|
| Exam- | | Starting | [% of | |
| ple | Structure | material | theory] | LC/MS |
| 94A | 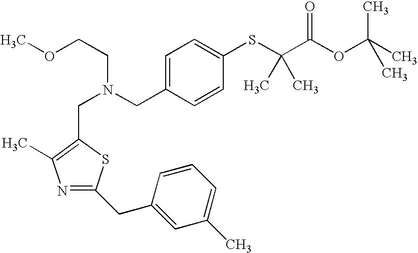 | Example 73A | 63% | Rₜ = 3.07 min; MS (ESIpos):m/z = 555 [M + H]⁺ (method 2) |
| 95A | 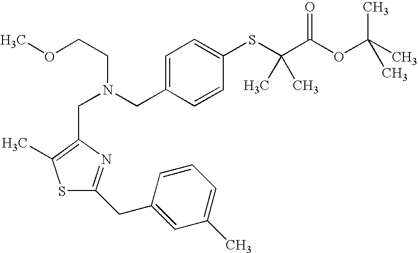 | Example 74A | 38% | |
| | ¹H-NMR (400 MHz, CDCl₃): δ = 1.41 (s, 6H), 1.42 (s, 9H), 2.28 (s, 3H), 2.32 (s, 3H), 2.72 (t, 2H), 3.28 (s, 3H), 3.49 (t, 2H), | | | |
| | 3.65 (s, 2H), 3.72 (s, 2H), 4.18 (s, 2H), 7.04-7.11 (m, 3H), 7.19 (m, 1H), 7.27 (m, 2H), 7.41 (d, 2H). | | | |
| 96A | 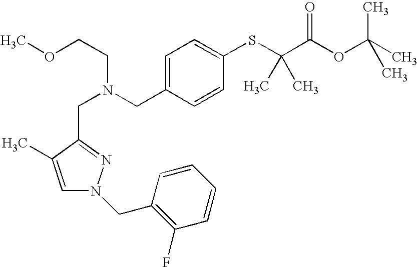 | Example 75A | 54% | Rₜ = 2.39 min; MS (ESIpos):m/z = 542 [M + H]⁺ (method 3) |

### Example 97A

### tert-Butyl 2-[(4-{[(2-methoxyethyl)(prop-2-yn-1-yl)amino]methyl}phenyl)thio]-2-methylpropionate

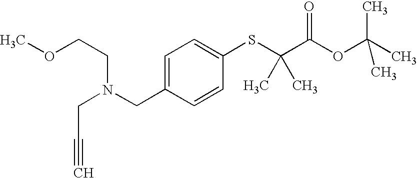

3.88 g (28.07 mmol) of potassium carbonate are added to a solution of 0.70 ml (9.36 mmol) of propargyl bromide and 3.52 g (9.36 mmol) of tert-butyl 2-[(4-{[(2-methoxyethyl)-amino]methyl}phenyl)thio]-2-methylpropionate (Example 92A) in 15.0 ml of anhydrous DMF, and the mixture is stirred at room temperature for one hour. Water is added, and the mixture is extracted twice with ethyl acetate. The combined organic phases are washed repeatedly with water and then once with saturated sodium chloride solution and dried over anhydrous magnesium sulphate. Chromatographic purification of the crude product (Biotage 40M, mobile phase: isohexane/ethyl acetate 80:20) gives 3.13 g (89% of theory) of the title compound as a colourless oil.

LC/MS (method 5): Rₜ=2.31 min; MS (ESIpos): m/z=378 [M+H]⁺

¹H-NMR (400 MHz, CDCl₃): δ=1.42 (s, 9H), 1.43 (s, 6H), 2.24 (t, 1H), 2.76 (t, 2H), 3.34 (s, 3H), 3.38 (d, 2H), 3.51 (t, 2H), 3.68 (s, 2H), 7.31 (d, 2H), 7.45 (d, 2H).

### Example 98A

### tert-Butyl 2-({4-[((2-methoxyethyl) {[1-(3-methylbenzyl)-1H-1,2,3-triazol-4-yl]methyl}amino)-methyl]phenyl}thio)-2-methylpropionate

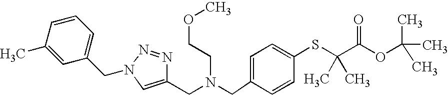

0.12 g (1.78 mmol) of sodium azide, 0.02 g (0.07 mmol) of copper(I) sulphate pentahydrate and 0.03 g (0.15 mmol) of sodium ascorbate are added successively to a solution of 0.56 g (1.48 mmol) of the compound from Example 97A and 0.2 ml (1.48 mmol) of 1-(bromomethyl)-3-methylbenzene in 2.4 ml of DMF and 0.6 ml of water. The mixture is stirred at room temperature for three hours and then poured into 40 ml of 2.5% strength ammonia solution. The aqueous phase is extracted with ethyl acetate. The organic phase is washed with saturated sodium chloride solution, dried over magnesium sulphate and concentrated under reduced pressure. Chromatographic purification of the crude product (Biotage 40M, mobile phase: isohexane/ethyl acetate 50:50→30:70) gives 0.48 g (62% of theory) of the title compound as a colourless oil.

LC/MS (method 5): Rₜ=2.37 min; MS (ESIpos): m/z=525 [M+H]⁺

¹H-NMR (400 MHz, DMSO-d₆): δ [ppm]=1.40 (s, 9H), 1.42 (s, 6H), 2.33 (s, 3H), 2.67 (t, 2H), 3.26 (s, 3H), 3.47 (t, 2H), 3.64 (s, 2H), 3.82 (s, 2H), 5.47 (s, 2H), 7.05 (d, 2H), 7.15 (d, 1H), 7.24 (d, 1H), 7.27 (d, 2H), 7.37 (s, 1H), 7.42 (d, 2H).

### Example 99A

### 2-(4-Fluorophenyl)ethane-N-hydroxyamidine

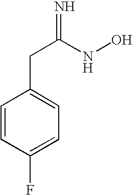

15.0 g (111 mmol) of 4-fluorophenylacetonitrile and 38.35 g (277 mmol) of potassium carbonate are initially charged in 250 ml of water/ethanol (10:1). 11.57 g (166 mmol) of hydroxylammonium chloride are then added. The mixture is stirred at room temperature overnight. The solvent is distilled off under reduced pressure, and saturated sodium chloride solution is added to the residue. The mixture is then extracted with dichloromethane, the combined organic phases are dried over sodium sulphate and the solvent is removed on a rotary evaporator. This gives 17.38 g (93% of theory) of the title compound.

LC/MS (method 6): Rₜ=0.60 min; MS (ESIpos): m/z=169 [M+H]⁺

¹H-NMR (400 MHz, DMSO-d₆): δ [ppm]=3.25 (s, 2H), 5.40 (br. s, 2H), 7.10 (t, 2H), 7.30 (dd, 2H), 8.90 (s, 1H).

### Example 100A

### 5-(Chloromethyl)-3-(4-fluorobenzyl)-1,2,4-oxadiazole

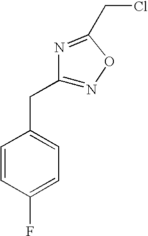

19.75 ml (248 mmol) of chloroacetyl chloride are added dropwise to a solution of 41.70 g (248 mmol) of the compound from Example 99A in 400 ml of DMF, and the mixture is stirred at 115° C. for 20 min. The solvent is distilled off under reduced pressure and the residue is purified by column filtration (silica gel, mobile phase: dichloromethane). This gives 34.00 g (59% of theory) of the title compound.

LC/MS (method 5): Rₜ=2.32 min; MS (ESIpos): m/z=227 [M+H]⁺

¹H-NMR (400 MHz, DMSO-d₆): δ [ppm]=4.14 (s, 2H), 5.07 (s, 2H), 7.17 (t, 2H), 7.36 (dd, 2H).

### Example 101A

### tert-Butyl 2-[(4-{[{[3-(4-fluorobenzyl)-1,2,4-oxadiazol-5-yl]methyl}(2-furylmethyl)amino]-methyl}phenyl)thio]-2-methylpropanoate

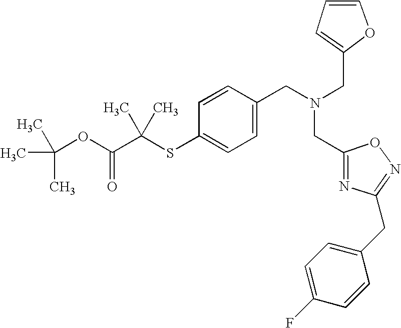

544 mg (1.37 mmol) of tert-butyl 2-[(4-{[(2-furylmethyl)amino]methyl}phenyl)thio]-2-methyl-propionate [WO 02/28821, Example II-3] are initially charged in 5 ml of DMF. 0.48 ml (3.42 mmol) of triethylamine, 101 mg (0.27 mmol) of tetra-n-butylammonium iodide, 0.24 ml (1.37 mmol) of N,N-diisopropylethylamine and 465 mg (2.05 mmol) of the compound from Example 100A are then added, and the mixture is stirred at 110° C. overnight. The solvent and the volatile components are removed on a rotary evaporator and the residue is then purified by preparative HPLC (mobile phase: acetonitrile/water with 0.1% formic acid, gradient 20:80→95:5). This gives 254 mg (34% of theory) of the title compound.

LC/MS (method 3): Rₜ=3.38 min; MS (ESIpos): m/z=552 [M+H]⁺

¹H-NMR (400 MHz, DMSO-d₆): δ [ppm]=1.32 (s, 9H), 1.36 (s, 6H), 3.71 (s, 4H), 3.90 (s, 2H), 4.10 (s, 2H), 6.27 (d, 1H), 6.38 (dd, 1H), 7.16 (t, 2H), 7.30-7.37 (m, 4H), 7.41 (d, 2H), 7.59 (d, 1H).

### Example 102A

### 4-(Chloromethyl)-N-[3-(trifluoromethyl)phenyl]-1,3-thiazole-2-amine

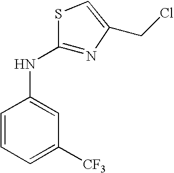

500 mg (2.27 mmol) of N-[3-(trifluoromethyl)phenyl]thiourea and 289 mg (2.27 mmol) of 1,3-di-chloroacetone in 5 ml of acetone are heated at reflux temperature for 6 h. The solvent is distilled off under reduced pressure and the residue is purified by preparative HPLC (mobile phase: acetonitrile/water with 0.1% formic acid, gradient 20:80→95:5). This gives 460 mg (69% of theory) of the title compound.

LC/MS (method 3): Rₜ=2.66 min; MS (ESIpos): m/z=293 [M+H]⁺

¹H-NMR (400 MHz, DMSO-d₆): δ [ppm]=4.69 (s, 2H), 7.05 (s, 1H), 7.29 (d, 1H), 7.55 (t, 1H), 7.83 (dd, 1H), 8.13 (br. s, 1H), 10.64 (s, 1H).

### Example 103A

### tert-Butyl 2-{[4-({(2-furylmethyl)[(2-{[3-(trifluoromethyl)phenyl]amino}-1,3-thiazol-4-yl)-methyl]amino}methyl)phenyl]thio}-2-methylpropanoate

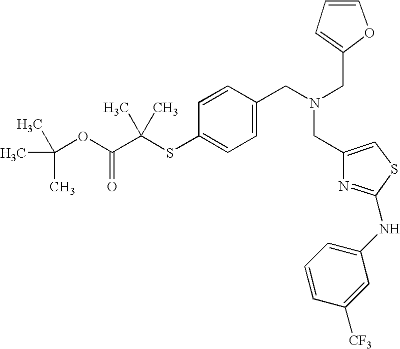

200 mg (0.50 mmol) of tert-butyl 2-[(4-{[(2-furylmethyl)amino]methyl}phenyl)thio]-2-methylpro-pionate [WO 02/28821, Example II-3] are initially charged in 5 ml of THF. 0.18 ml (1.26 mmol) of triethylamine, 37 mg (0.10 mmol) of tetra-n-butylammonium iodide and 221 mg (0.75 mmol) of the compound from Example 102A are then added. The mixture is stirred at 90° C. overnight and then at 110° C. for 2 h. The solvent and the volatile components are removed on a rotary evaporator and the residue is then purified by preparative HPLC (mobile phase: acetonitrile/water with 0.1% formic acid, gradient 20:80→95:5). This gives 120 mg (39% of theory) of the title compound.

LC/MS (method 3): Rₜ=3.01 min; MS (ESIpos): m/z=618 [M+H]⁺

¹H-NMR (400 MHz, DMSO-d₆): δ [ppm]=1.30 (s, 9H), 1.36 (s, 6H), 3.58 (s, 2H), 3.69 (s, 4H), 6.34 (d, 1H), 6.41 (dd, 1H), 6.77 (s, 1H), 7.26 (d, 1H), 7.41 (s, 4H), 7.52 (t, 1H), 7.61 (d, 1H), 7.72 (s, 1H), 8.34 (s, 1H), 10.52 (s, 1H).

### Example 104A

### Ethyl 2-(3-chlorophenoxy)thiazole-5-carboxylate

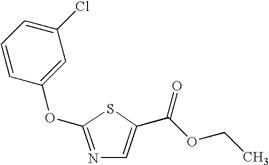

1.9 g (8.05 mmol) of ethyl 2-bromothiazole-5-carboxylate, 1.14 g (8.85 mmol) of 3-chlorophenol and 2.22 g (16.1 mmol) of potassium carbonate in 9.5 ml of DMF are stirred at 80° C. for three hours. After cooling, the mixture is poured into water and extracted with ethyl acetate. The combined organic phases are washed with 1 M aqueous sodium hydroxide solution, dried over potassium carbonate and magnesium sulphate and concentrated. The crude product is purified chromatographically (silica gel, mobile phase: dichloromethane/ethanol 100:1). This gives 1.95 g (85% of theory) of the title compound.

LC/MS (method 2): Rₜ=2.61 min; MS (ESIpos): m/z=284 [M+H]⁺

¹H-NMR (400 MHz, CDCl₃): δ [ppm]=1.35 (t, 3H), 4.35 (q, 2H), 7.2-7.45 (m, 4H), 7.9 (s, 1H).

### Example 105A

### [2-(3-Chlorophenoxy)thiazol-5-yl]methanol

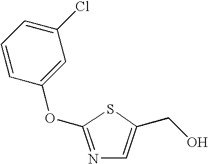

At −10° C., 1.9 g (6.72 mmol) of the compound from Example 104A are initially charged in 8 ml of absolute THF, and 4.03 ml (4.03 mmol) of a 1 M lithium aluminium hydride solution in THF are added dropwise. The mixture is stirred at −10° C. for another hour, and 0.17 ml of water, 0.17 ml of 15% strength aqueous potassium hydroxide solution and 0.17 ml of water are then successively added dropwise at 0° C. The precipitate is filtered off with suction and the filtrate is concentrated. Water is added to the residue, the mixture is extracted with ethyl acetate and the organic phase is dried over magnesium sulphate. The crude product is purified chromatographically (silica gel, mobile phase: dichloromethane/ethanol 50:1). This gives 940 mg (53% of theory) of the title compound.

LC/MS (method 5): Rₜ=2.13 min; MS (ESIpos): m/z=242 [M+H]⁺

¹H-NMR (400 MHz, DMSO-d₆): δ [ppm]=4.6 (d, 2H), 5.5 (t, 1H), 7.15 (s, 1H), 7.35 (dd, 1H), 7.4 (dd, 1H), 7.5 (m, 2H).

### Example 106A

### 5-Chloromethyl-2-(3-chlorophenoxy)thiazole

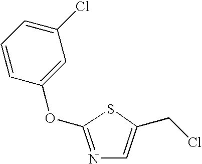

At 0° C., 935 mg (3.87 mmol) of the compound from Example 105A and 614 mg (5 mmol) of 4-N,N-dimethylaminopyridine are initially charged in 12 ml of dichloromethane, and 885 mg (4.6 mmol) of p-toluenesulphonyl chloride are added. The mixture is stirred at room temperature for two hours, another 61 mg of 4-N,N-dimethylaminopyridine and another 88 mg of p-toluenesulphonyl chloride are then added and the mixture is stirred at room temperature for another two hours. All volatile components are removed under reduced pressure and the crude product is purified chromatographically (silica gel, mobile phase: dichloromethane/ethanol 200:1). This gives 512 mg (50% of theory) of the title compound.

LC/MS (method 3): Rₜ=2.00 min

MS (DC₁, NH₃): m/z=277 [M+NH₄]⁺, 260 [M+H]⁺

¹H-NMR (400 MHz, CDCl₃): δ [ppm]=4.7 (s, 2H), 7.15-7.4 (m, 5H).

### Example 107A

### tert-Butyl 2-(4-{[[2-(3-chlorophenoxy)thiazol-5-ylmethyl] (2-methoxyethyl)amino]methyl}phenyl-thio)-2-methylpropanoate

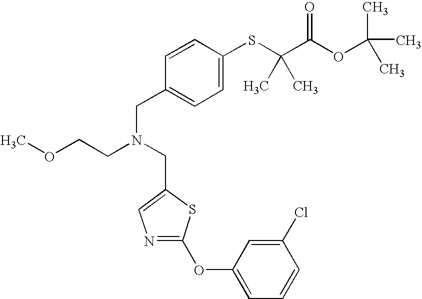

130 mg (0.38 mmol) of the compound from Example 92A, 100 mg (0.38 mmol) of the compound from Example 106A and 106 mg (0.77 mmol) of potassium carbonate in 1 ml of DMF are heated at 90° C. for six hours. After cooling, the mixture is added to water and extracted with ethyl acetate. The combined organic phases are washed with saturated sodium chloride solution, dried over sodium sulphate and concentrated. The crude product is purified chromatographically (silica gel, mobile phase: dichloromethane/ethanol 100:1, 50:1, 20:1). This gives 70 mg (32% of theory) of the title compound.

LC/MS (method 5): Rₜ=3.35 min; MS (ESIpos): m/z=563 [M+H]⁺

¹H-NMR (400 MHz, CDCl₃): δ [ppm]=1.40 (s, 9H), 1.45 (s, 6H), 2.7 (t, 2H), 3.3 (s, 3H), 3.5 (t, 2H), 3.70 (s, 2H), 3.75 (s, 2H), 7.0 (s, 1H), 7.15-7.4 (m, 6H), 7.45 (d, 2H).

### WORKING EXAMPLES

### Example 1

### 2-{[4-(((2-Furylmethyl)[(3-phenyl-1,2,4-oxadiazol-5-yl)methyl]amino}methyl)phenyl]thio)-2-methylpropionic acid

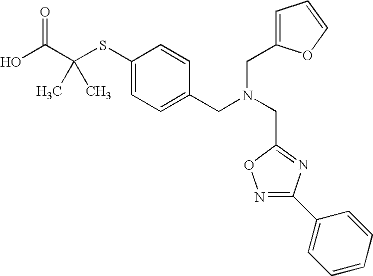

113 mg of the compound from Example 6A (0.22 mmol) are dissolved in 3 ml of a 4 M solution of hydrogen chloride gas in dioxane and stirred at room temperature for 16 h. The solvent is removed on a rotary evaporator and the residue is purified by preparative HPLC (mobile phase: acetonitrile/water with 0.1% formic acid, gradient 20:80→95:5). This gives 76 mg (75% of theory) of the title compound.

¹H-NMR (400 MHz, CDCl₃): δ=1.51 (s, 6H), 4.10 (br. s, 6H), 6.35 (br. s, 1H), 6.46 (br. s, 1H), 7.40 (br. s, 1H), 7.48-7.55 (m, 7H), 8.09-8.11 (m, 2H).

LC/MS (method 2): Rₜ=2.74 min; MS (ESIpos): m/z=464 [M+H]⁺.

### Example 2

### 2-[(4-{[[3-(2,4-Dimethylphenyl)-1,2,4-oxadiazol-5-yl](2-furylmethyl)amino]methyl}phenyl)thio]-2-methylpropionic acid

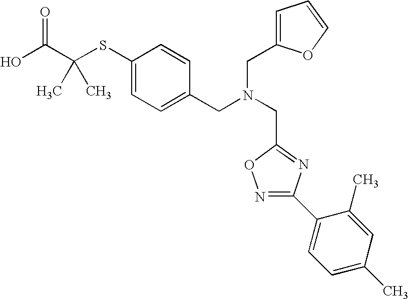

275 mg of the compound from Example 5A (0.50 mmol) are dissolved in 7 ml of a 4 M solution of hydrogen chloride gas in dioxane and stirred at room temperature for 16 h. The solvent is removed on a rotary evaporator and the residue is purified by preparative HPLC (mobile phase: acetonitrile/water with 0.1% formic acid, gradient 20:80→95:5). This gives 205 mg (83% of theory) of the title compound.

¹H-NMR (400 MHz, CDCl₃): δ=1.50 (s, 6H), 2.38 (s, 3H), 2.61 (s, 3H), 3.82 (s, 2H), 3.85 (s, 2H), 3.99 (s, 2H), 6.27-6.28 (m, 1H), 6.32 (dd, J=3.2, J=1.9, 1H), 7.11-7.13 (m, 2H), 7.38-7.40 (m, 3H), 7.49-7.52 (m, 2H), 7.91 (d, J=8.5, 1H).

LC/MS (method 5): Rₜ=3.15 min; MS (ESIpos): m/z=492 [M+H]⁺.

### Example 3

### 2-({4-[((2-Furylmethyl){[3-(4-methoxy-2-methylphenyl)isoxazol-5-yl]methyl}amino)methyl]-phenyl}thio)-2-methylpropionic acid

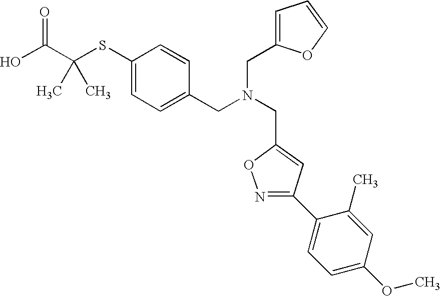

70 mg of the compound from Example 10A (0.12 mmol) are dissolved in 3 ml a 4 M solution of hydrogen chloride gas in dioxane and stirred at room temperature for 16 h. The solvent is distilled off on a rotary evaporator and the residue is purified by preparative HPLC (mobile phase: acetonitrile/water with 0.1% formic acid, gradient 20:80→95:5). This gives 25 mg (39% of theory) of the title compound.

¹H-NMR (400 MHz, CDCl₃): δ=1.51 (s, 6H), 2.47 (s, 3H), 3.73 (s, 2H), 3.75 (s, 2H), 3.84 (s, 5H), 6.25-6.27 (m, 1H), 6.33-6.35 (m, 2H), 6.78-6.83 (m, 2H), 7.37-7.50 (m, 6H).

LC/MS (method 5): Rₜ=2.97 min; MS (ESIpos): m/z=507 [M+H]⁺.

### Example 4

### 2-[(4-{[({3-[2,4-Bis(trifluoromethyl)phenyl]isoxazol-5-yl}methyl)(2-furylmethyl)amino]methyl}-phenyl)thio]-2-methylpropionic acid

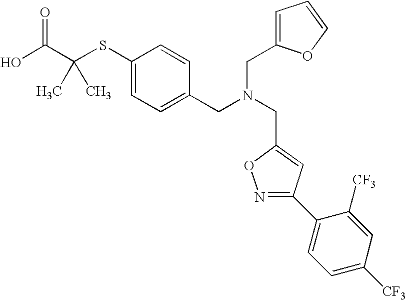

39 mg of the compound from Example 11A (0.12 mmol) are dissolved in 3 ml of a 4 M solution of hydrogen chloride gas in dioxane and stirred at room temperature for 16 h. The solvent is distilled off on a rotary evaporator and the residue is purified by preparative HPLC (mobile phase: acetonitrile/water with 0.1% formic acid, gradient 20:80→95:5). This gives 32 mg (91% of theory) of the title compound.

¹H-NMR (400 MHz, CDCl₃): δ=1.51 (s, 6H), 3.77 (s, 2H), 3.80 (s, 2H), 3.91 (s, 2H), 6.29-6.31 (m, 1H), 6.35-6.37 (m, 1H), 6.50 (br. s, 1H), 7.40-7.50 (m, 5H), 7.81-7.92 (m, 2H), 8.06 (br. s, 1H).

LC/MS (method 4): Rₜ=3.19 min; MS (ESIpos): m/z=599 [M+H]⁺.

### Example 5

### 2-[(4-{[{[3-(2,4-Difluorophenyl)-1,2,4-oxadiazol-5-yl]methyl}(2-furylmethyl)amino]methyl}-phenyl)thio]-2-methylpropionic acid

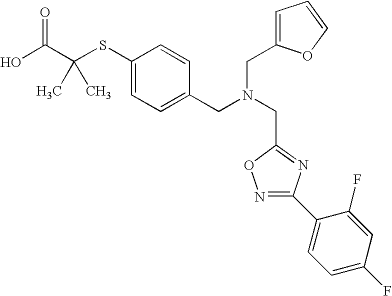

The title compound was prepared analogously to Example 2.

¹H-NMR (300 MHz, DMSO-d₆): δ=1.36 (s, 6H), 3.80 (s, 4H), 4.05 (s, 2H), 6.34-6.36 (m, 1H), 6.38-6.41 (m, 1H), 7.28-7.36 (m, 1H), 7.39 (m, 4H), 7.48-7.56 (m, 1H), 7.59-7.61 (m, 1H), 8.08 (m, 1H), 12.54 (br. s, 1H).

MS (ESIpos): m/z=500 [M+H]⁺

HPLC (method 1): Rₜ=4.90 min

### Example 6

### 2-({4-[((2-Furylmethyl){([3-(3-methylphenyl)-1,2,4-oxadiazol-5-yl]methyl}amino)methyl]phenyl}-thio)-2-methylpropionic acid

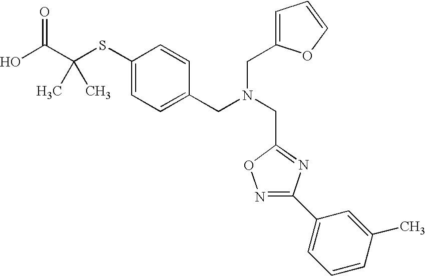

The title compound was prepared analogously to Example 2.

¹H-NMR (300 MHz, DMSO-d₆): δ=1.36 (s, 6H), 2.41 (s, 3H), 3.80 (s, 4H), 4.03 (s, 2H), 6.34-6.37 (m, 1H), 6.39-6.42 (m, 1H), 7.35-7.49 (m, 6H), 7.60-7.62 (m, 1H), 7.78-7.84 (m, 2H), 12.54 (br. s, 1H).

MS (ESIpos): m/z=478 [M+H]⁺

HPLC (method 1): Rₜ=5.02 min

### Example 7

### 2-[(4-{[{[3-(2,4-Dimethylphenyl)-1,2,4-oxadiazol-5-yl]methyl}(2-methoxyethyl)amino]methyl}-phenyl)thio]-2-methylpropionic acid

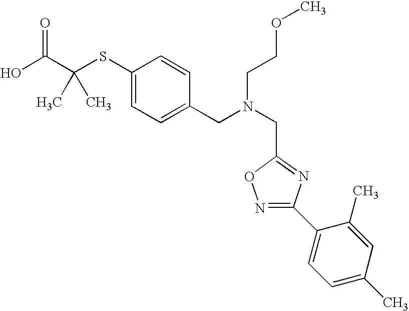

42.2 mg of the compound from Example 14A (0.0803 mmol) are dissolved in 3 ml of a 4 M solution of hydrogen chloride gas in dioxane and stirred at room temperature overnight. The solvent is removed under reduced pressure and the residue is purified by preparative HPLC (mobile phase: acetonitrile/water with 0.1% formic acid, gradient 20:80→95:5). This gives 33.7 mg (89% of theory) of the title compound.

¹H-NMR (300 MHz, DMSO-d₆): δ=1.36 (s, 6H), 2.34 (s, 3H), 2.52 (s, 3H), 2.80 (t, 2H), 3.19 (s, 3H), 3.47 (t, 2H), 3.83 (s, 2H), 4.11 (s, 2H), 7.19 (d, 1H), 7.22 (s, 1H), 7.39 (m, 4H), 7.81 (d, 1H), 12.53 (br. s, 1H).

MS (ESIpos): m/z=470 [M+H]⁺

HPLC (method 1): Rₜ=4.67 min

### Example 8

### 2-[(4-{[{[3-(2,4-Dimethylphenyl)-1,2,4-oxadiazol-5-yl]methyl}(2-furylmethyl)amino]methyl}-phenyl)thio]-N,2-dimethylpropionamide

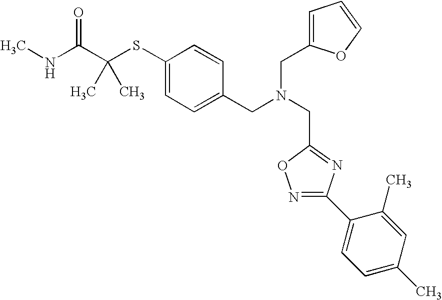

58 mg of PyBOP (0.11 mmol) and 19 μl of N,N-diisopropylethylamine (14 mg, 0.11 mmol) are added to 50 mg of the compound from Example 2 (0.10 mmol) in 5 ml of tetrahydrofuran and 20 μl of dimethylformamide, and the mixture is stirred at room temperature for 1 h. 56 μl of methylamine (3.5 mg, 0.11 mmol) are then added, and the reaction mixture is stirred further at room temperature overnight. The solvent is removed under reduced pressure and the residue is purified by preparative HPLC (mobile phase: acetonitrile/water with 0.1% formic acid, gradient 20:80→95:5). This gives 41 mg (79% of theory) of the title compound.

¹H-NMR (300 MHz, CDCl₃): δ=1.49 (s, 6H), 2.38 (s, 3H), 2.61 (s, 3H), 2.84 (d, 3H), 3.81 (s, 2H), 3.86 (s, 2H), 3.99 (s, 2H), 6.27-6.30 (m, 1H), 6.31-6.35 (m, 1H), 6.82-6.89 (m, 1H), 7.10-7.15 (m, 2H), 7.31-7.41 (m, 5H), 7.91 (d, 1H).

MS (ESIpos): m/z=505 [M+H]⁺

HPLC (method 1): Rₜ=5.05 min

### Example 9

### 2-{[4-({(2-Furylmethyl)[(5-phenyl-1,3,4-oxadiazol-2-yl)methyl]amino}methyl)phenyl]thio}-2-methylpropionic acid

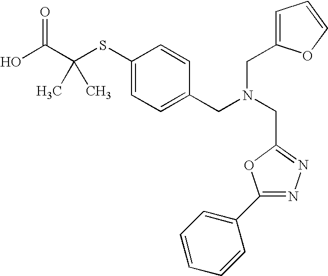

165 mg of the compound from Example 15A (0.318 mmol) are dissolved in 5 ml of a 4 M solution of hydrogen chloride gas in dioxane and stirred at room temperature overnight. The solvent is removed under reduced pressure and the residue is purified by preparative HPLC (mobile phase: acetonitrile/water with 0.1% formic acid, gradient 20:80→95:5). This gives 127 mg (86% of theory) of the title compound.

¹H-NMR (300 MHz, DMSO-d₆): δ=1.35 (s, 6H), 3.77 (s, 2H), 3.79 (s, 2H), 3.97 (s, 2H), 6.35-6.38 (m, 1H), 6.39-6.42 (m, 1H), 7.39 (m, 4H), 7.57-7.65 (m, 4H), 7.95-8.01 (m, 2H), 12.53 (br. s, 1H).

MS (ESIpos): m/z=464 [M+H]⁺

HPLC (method 1): Rₜ=4.47 min

### Example 10

### 2-[(4-{[{[5-(4-Chlorophenyl)-1,3,4-oxadiazol-2-yl]methyl}(2-furylmethyl)amino]methyl}phenyl)-thio]-2-methylpropionic acid

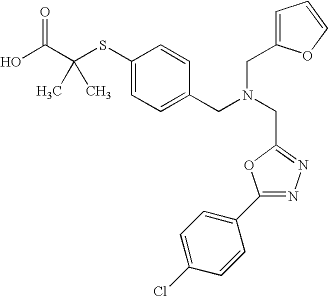

The title compound is prepared analogously to Example 9.

¹H-NMR (400 MHz, DMSO-d₆): δ=1.35 (s, 6H), 3.77 (s, 2H), 3.79 (s, 2H), 3.96 (s, 2H), 6.34-6.37 (m, 1H), 6.38-6.41 (m, 1H), 7.38 (m, 4H), 7.60-7.61 (m, 1H), 7.66-7.71 (m, 2H), 7.95-8.01 (m, 2H), 12.53 (br. s, 1H).

MS (ESIpos): m/z=498 [M+H]⁺

HPLC (method 1): Rₜ=4.72 min

### Example 11

### 2-({4-[((2-Furylmethyl){[5-(4-methoxyphenyl)-1,3,4-oxadiazol-2-yl]methyl}amino)methyl]-phenyl}thio)-2-methylpropionic acid

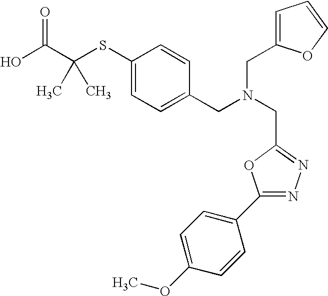

The title compound is prepared analogously to Example 9.

¹H-NMR (300 MHz, DMSO-d₆): δ=1.36 (s, 6H), 3.77 (s, 2H), 3.78 (s, 2H), 3.86 (s, 3H), 3.94 (s, 2H), 6.35-6.37 (m, 1H), 6.39-6.42 (m, 1H), 7.12-7.19 (m, 2H), 7.39 (m, 4H), 7.60-7.62 (m, 1H), 7.88-7.94 (m, 2H), 12.54 (br. s, 1H).

MS (ESIpos): m/z=494 [M+H]⁺

HPLC (method 1): Rₜ=4.45 min

### Example 12

### 2-[(4-{[{[5-(4-Fluorophenyl)-1,3,4-oxadiazol-2-yl]methyl}(2-furylmethyl)amino]methyl}phenyl)-thio]-2-methylpropionic acid

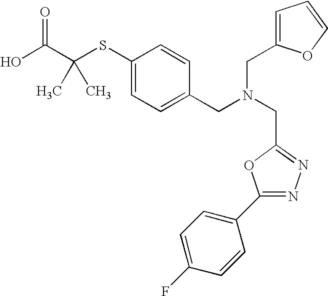

The title compound is prepared analogously to Example 9.

¹H-NMR (300 MHz, DMSO-d₆): δ=1.35 (s, 6H), 3.77 (s, 2H), 3.79 (s, 2H), 3.96 (s, 2H), 6.34-6.37 (m, 1H), 6.39-6.42 (m, 1H), 7.34-7.41 (m, 4H), 7.41-7.50 (m, 2H), 7.60-7.62 (m, 1H), 7.99-8.06 (m, 2H), 12.52 (br. s, 1H).

MS (ESIpos): m/z=482 [M+H]⁺

HPLC (method 1): Rₜ=4.53 min

### Example 13

### 2-[(4-{[{[3-(4-Fluorobenzyl)-1,2,4-oxadiazol-5-yl]methyl}(2-furylmethyl)amino]methyl}phenyl)-thio]-2-methylpropionic acid

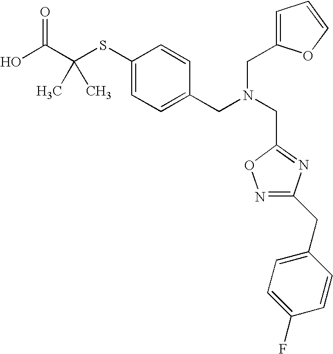

134 mg of the compound from Example 17A (0.242 mmol) are dissolved in 3 ml of a 4 M solution of hydrogen chloride gas in dioxane and stirred at room temperature overnight. The solvent is removed under reduced pressure and the residue is purified by preparative HPLC (mobile phase: acetonitrile/water with 0.1% formic acid, gradient 20:80→95:5). This gives 108 mg (89% of theory) of the title compound.

¹H-NMR (400 MHz, DMSO-d₆): δ=1.36 (s, 6H), 3.71 (s, 2H), 3.91 (s, 2H), 4.09 (s, 2H), 6.27-6.29 (m, 1H), 6.37-6.49 (m, 1H), 7.16 (m, 2H), 7.29-7.37 (m, 4H), 7.38-7.42 (m, 2H), 7.58-7.60 (m, 1H), 12.60 (br. s, 1H).

MS (ESIpos): m/z=496 [M+H]⁺

HPLC (method 7): Rₜ=4.82 min

### Example 14

### 2-({4-[((2-Furylmethyl){[3-(4-methylbenzyl)-1,2,4-oxadiazol-5-yl]methyl}amino)methyl]phenyl}-thio)-2-methylpropionic acid

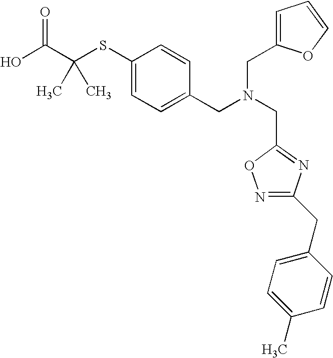

The title compound is prepared analogously to Example 13.

¹H-NMR (400 MHz, DMSO-d₆): δ=1.37 (s, 6H), 2.70 (s, 3H), 3.696 (s, 2H), 3.702 (s, 2H), 3.90 (s, 2H), 4.02 (s, 2H), 6.27-6.29 (m, 1H), 6.37-6.39 (m, 1H), 7.13 (d, 2H), 7.18 (d, 2H), 7.31 (d, 2H), 7.40 (d, 2H), 7.58-7.60 (m, 1H), 12.59 (br. s, 1H).

MS (ESIpos): m/z=492 [M+H]⁺

HPLC (method 7): Rₜ=4.95 min

### Example 15

### 2-({4-[((2-Furylmethyl) {[3-(3-methylbenzyl)-1,2,4-oxadiazol-5-yl]methyl}amino)methyl]phenyl}-thio)-2-methylpropionic acid

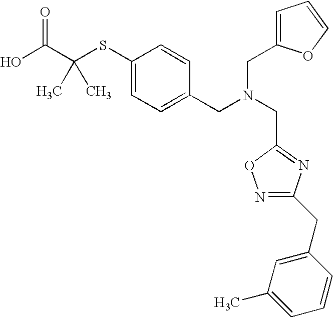

The title compound is prepared analogously to Example 13.

¹H-NMR (400 MHz, DMSO-d₆): δ=1.37 (s, 6H), 2.27 (s, 3H), 3.70 (s, 4H), 3.91 (s, 2H), 4.03 (s, 2H), 6.27-6.29 (m, 1H), 6.37-6.39 (m, 1H), 7.05-7.10 (m, 2H), 7.11 (s, 1H), 7.22 (dd, 1H), 7.31 (d, 2H), 7.40 (d, 2H), 7.58-7.60 (m, 1H) 12.60 (br. s, 1H).

MS (ESIpos): m/z=492 [M+H]⁺

HPLC (method 7): Rₜ=4.94 min

### Example 16

### 2-[(4-{[{[3-(2,4-Difluorobenzyl)-1,2,4-oxadiazol-5-yl]methyl}(2-furylmethyl)amino]methyl}-phenyl)thio]-2-methylpropionic acid

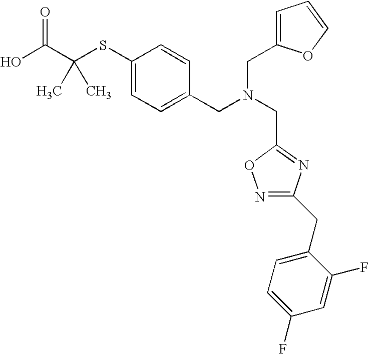

The title compound is prepared analogously to Example 13.

¹H-NMR (300 MHz, DMSO-d₆): δ=1.36 (s, 6H), 3.70 (s, 4H), 3.91 (s, 2H), 4.11 (s, 2H), 6.26-6.29 (m, 1H), 6.36-6.39 (m, 1H), 7.05-7.10 (m, 1H), 7.22-7.34 (m, 3H), 7.37-7.50 (m, 3H), 7.58-7.60 (m, 1H), 12.60 (br. s, 1H).

MS (ESIpos): m/z=514 [M+H]⁺

HPLC (method 1): Rₜ=4.95 min

### Example 17

### 2-[(4-{[{[3-(2,4-Dimethylbenzyl)-1,2,4-oxadiazol-5-yl]methyl}(2-furylmethyl)amino]methyl}-phenyl)thio]-2-methylpropionic acid

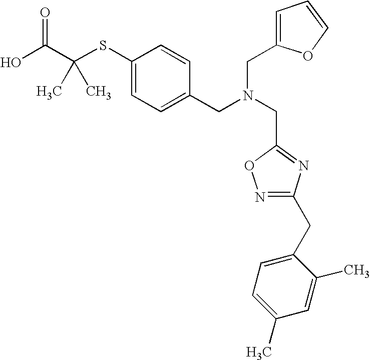

The title compound is prepared analogously to Example 13.

¹H-NMR (400 MHz, DMSO-d₆): δ=1.37 (s, 6H), 2.24 (s, 3H), 2.25 (s, 3H), 3.689 (s, 2H), 3.698 (s, 2H), 3.89 (s, 2H), 4.00 (s, 2H), 6.26-6.29 (m, 1H), 6.37-6.39 (m, 1H), 6.95 (d, 1H), 7.00 (s, 1H), 7.06 (d, 1H), 7.30 (d, 2H), 7.39 (d, 2H), 7.58 (s, 1H).

MS (ESIpos): m/z=506 [M+H]⁺

HPLC (method 1): Rₜ=5.16 min

### Example 18

### 2-({4-[((2-Furylmethyl) {[3-(2-methylbenzyl)-1,2,4-oxadiazol-5-yl]methyl}amino)methyl]phenyl}-thio)-2-methylpropionic acid

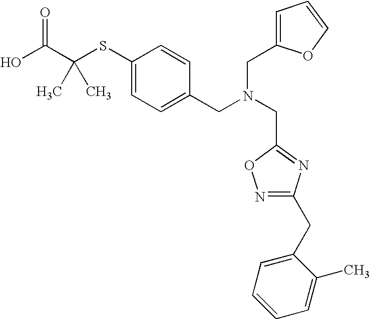

The title compound is prepared analogously to Example 13.

¹H-NMR (400 MHz, DMSO-d₆): δ=1.37 (s, 6H), 2.30 (s, 3H), 3.70 (s, 4H), 3.90 (s, 2H), 4.06 (s, 2H), 6.26-6.28 (m, 1H), 6.36-6.39 (m, 1H), 7.13-7.21 (m, 4H), 7.30 (d, 2H), 7.40 (d, 2H), 7.58 (s, 1H), 12.59 (br. s, 1H).

MS (ESIpos): m/z=492 [M+H]⁺

HPLC (method 1): Rₜ=4.99 min

### Example 19

### 2-({4-[((2-Furylmethyl) {[5-(4-methoxyphenyl)-1,2,4-oxadiazol-3-yl]methyl}amino)methyl]-phenyl}thio)-2-methylpropionic acid

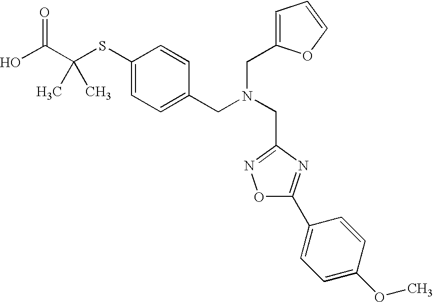

33 mg of the compound from Example 18A (0.061 mmol) are dissolved in 1 ml of a 4 M solution of hydrogen chloride gas in dioxane and stirred at room temperature overnight. The solvent is removed under reduced pressure and the residue is purified by preparative HPLC (mobile phase: acetonitrile/water with 0.1% formic acid, gradient 20:80→95:5). This gives 24 mg (81% of theory) of the title compound.

¹H-NMR (400 MHz, DMSO-d₆): δ=1.36 (s, 6H), 3.73 (s, 2H), 3.77 (s, 2H), 3.80 (s, 2H), 3.88 (s, 3H), 6.36-6.38 (m, 1H), 6.42-6.44 (m, 1H), 7.18 (d, 2H), 7.40 (m, 4H), 7.64 (s, 1H), 8.07 (d, 2H), 12.50 (br. s, 1H).

MS (ESIpos): m/z=494 [M+H]⁺

HPLC (method 1): Rₜ=4.52 min

### Example 20

### 2-[(4-{[{[5-(3,4-Dichlorophenyl)-1,2,4-oxadiazol-3-yl]methyl}(2-furylmethyl)amino]methyl}-phenyl)thio]-2-methylpropionic acid

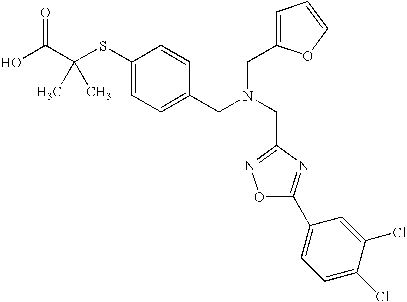

The title compound is prepared analogously to Example 19.

¹H-NMR (400 MHz, DMSO-d₆): δ=1.35 (s, 6H), 3.75 (s, 2H), 3.77 (s, 2H), 3.84 (s, 2H), 6.36-6.38 (m, 1H), 6.41-6.43 (m, 1H), 7.39 (m, 4H), 7.62-7.64 (m, 1H), 7.93 (d, 1H), 8.09 (dd, 1H), 8.30 (d, 1H), 12.59 (br. s, 1H).

MS (ESIpos): m/z=532 [M+H]⁺

HPLC (method 1): Rₜ=4.87 min

### Example 21

### 2-{[4-({(2-Furylmethyl) [(5-phenyl-1,2,4-oxadiazol-3-yl)methyl]amino}methyl)phenyl]thio}-2-methylpropionic acid

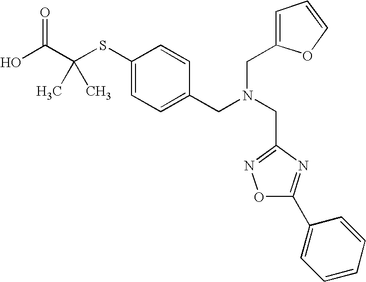

The title compound is prepared analogously to Example 19.

¹H-NMR (400 MHz, DMSO-d₆): δ=1.36 (s, 6H), 3.56 (s, 2H), 3.58 (s, 2H), 3.85 (s, 2H), 6.38-6.40 (m, 1H), 6.42-6.46 (m, 1H), 7.41 (m, 4H), 7.63-7.69 (m, 3H), 7.70-7.77 (m, 1H), 8.14 (d, 2H), 12.49 (br. s, 1H).

MS (ESIpos): m/z=464 [M+H]⁺

HPLC (method 1): Rₜ=4.49 min

### Example 22

### 2-({4-[((2-Furylmethyl) {[5-(2-methoxyphenyl)-1,2,4-oxadiazol-3-yl]methyl}amino)methyl]-phenyl}thio)-2-methylpropionic acid

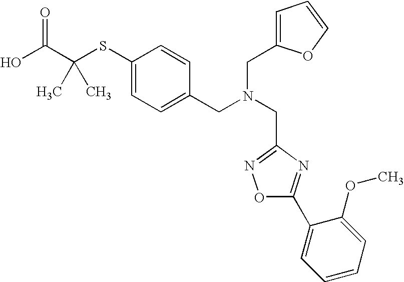

The title compound is prepared analogously to Example 19.

¹H-NMR (400 MHz, DMSO-d₆): δ=1.36 (s, 6H), 3.72 (s, 2H), 3.76 (s, 2H), 3.83 (s, 2H), 3.85 (s, 3H), 6.37-6.39 (m, 1H), 6.41-6.44 (m, 1H), 7.17 (dd, 1H), 7.30 (d, 1H), 7.41 (m, 4H), 7.65 (s, 1H), 7.68 (dd, 1H), 8.00 (dd, 1H), 12.60 (br. s, 1H).

MS (ESIpos): m/z=494 [M+H]⁺

HPLC (method 1): Rₜ=4.44 min

### Example 23

### 2-[(4-{[(2-Furylmethyl)({5-[4-(trifluoromethyl)phenyl]-1,2,4-oxadiazol-3-yl}methyl)amino]-methyl}phenyl)thio]-2-methylpropionic acid

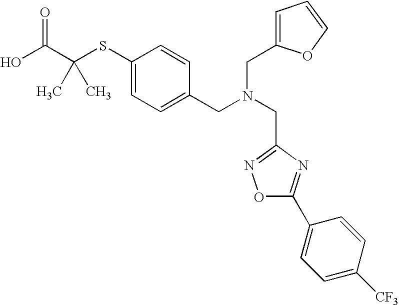

The title compound is prepared analogously to Example 19.

¹H-NMR (400 MHz, DMSO-d₆): δ=1.37 (s, 6H), 3.74 (s, 2H), 3.76 (s, 2H), 3.87 (s, 2H), 6.47-6.49 (m, 1H), 6.40-6.45 (m, 1H), 7.38 (m, 4H), 7.64 (s, 1H), 8.02 (d, 2H), 8.34 (d, 2H), 12.58 (br. s, 1H).

MS (ESIpos): m/z=532 [M+H]⁺

HPLC (method 1): Rₜ=4.78 min

### Example 24

### 2-[(4-{[(2-Furylmethyl)({5-[3-(trifluoromethyl)phenyl]-1,2,4-oxadiazol-3-yl}methyl)amino]-methyl}phenyl)thio]-2-methylpropionic acid

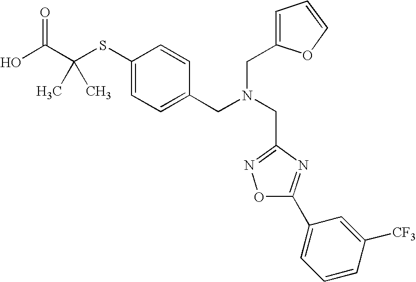

The title compound is prepared analogously to Example 19.

¹H-NMR (400 MHz, DMSO-d₆): δ=1.34 (s, 6H), 3.75 (s, 2H), 3.77 (s, 2H), 3.86 (s, 2H), 6.36-6.38 (m, 1H), 6.40-6.43 (m, 1H), 7.37 (m, 4H), 7.62 (s, 1H), 7.89 (dd, 1H), 8.10 (d, 1H), 8.32 (s, 1H), 8.41 (d, 1H), 12.57 (br. s, 1H).

MS (ESIpos): m/z=532 [M+H]⁺

HPLC (method 1): Rₜ=4.74 min

### Example 25

### 2-[(4-{[{[1-(3,5-Dichlorophenyl)-5-methyl-1H-pyrazol-3-yl]methyl}(2-furylmethyl)amino]-methyl}phenyl)thio]-2-methylpropionic acid

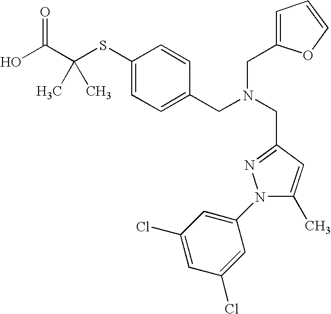

The title compound is prepared analogously to Example 19.

¹H-NMR (400 MHz, DMSO-d₆): δ=1.37 (s, 6H), 2.41 (s, 3H), 3.57 (s, 2H), 3.59 (s, 2H), 3.62 (s, 2H), 6.32-6.35 (m, 2H), 6.40-6.43 (m, 1H), 7.39 (m, 4H), 7.63-7.66 (m, 4H), 12.58 (br. s, 1H).

MS (ESIpos): m/z=544 [M+H]⁺

HPLC (method 1): Rₜ=4.85 min

### Example 26

### 2-{[4-({(2-Furylmethyl)[(3-methyl-1-phenyl-1H-pyrazol-4-yl)methyl]amino}methyl)phenyl]thio}-2-methylpropionic acid

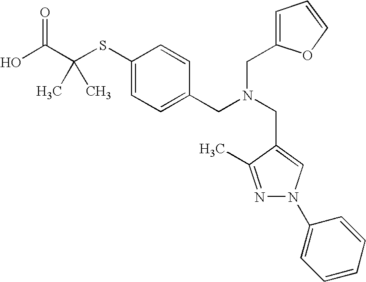

The title compound is prepared analogously to Example 19 starting with 4-chloromethyl-3-methyl-1-phenyl-1H-pyrazole [preparation, for example, according to Grandberg et al., J. Gen. Chem. USSR (Engl. Transl.) 30, 3292 (1960); Perez et al., Heterocycles 60 (1), 167-176 (2003)].

¹H-NMR (400 MHz, CDCl₃): δ=1.50 (s, 6H), 2.26 (s, 3H), 3.52 (s, 2H), 3.61 (s, 2H), 3.66 (s, 2H), 6.17-6.19 (m, 1H), 6.32-6.36 (m, 1H), 7.23 (dd, 1H), 7.34 (d, 2H), 7.37-7.44 (m, 3H), 7.47 (d, 2H), 7.64 (d, 2H), 7.84 (s, 1H).

MS (ESIpos): m/z=476 [M+H]⁺

HPLC (method 1): Rₜ=4.44 min

### Example 27

### 2-{[4-({(2-Furylmethyl)[(1-phenyl-1H-pyrazol-4-yl)methyl]amino}methyl)phenyl]thio}-2-methylpropionic acid

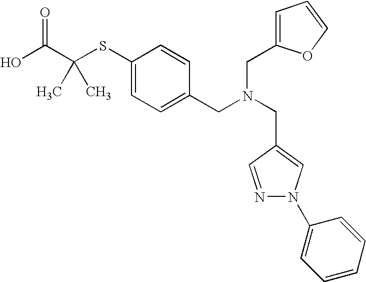

The title compound is prepared analogously to Example 19 starting with 4-chloromethyl-1-phenyl-1H-pyrazole [preparation, for example, according to Finar et al., J. Chem. Soc., 2293-2295 (1954)].

¹H-NMR (400 MHz, DMSO-d₆): δ=1.49 (s, 6H), 4.19-4.43 (m, 6H), 6.55-6.60 (m, 1H), 6.76-6.80 (m, 1H), 7.32-7.39 (m, 1H), 7.47-7.62 (m, 6H), 7.80-7.86 (m, 3H), 7.92 (s, 1H), 8.66 (s, 1H), 12.69 (br. s, 1H).

MS (ESIpos): m/z=462 [M+H]⁺

HPLC (method 1): Rₜ=4.39 min

### Example 28

### 2-[(4-{[(2-Furylmethyl)({4-methyl-2-[4-(trifluoromethyl)phenyl]-1,3-thiazol-5-yl}methyl)amino]-methyl}phenyl)thio]-2-methylpropionic acid

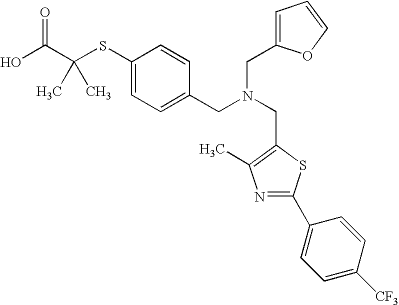

The title compound is prepared analogously to Example 19 starting with 5-chloromethyl-4-methyl-2-(4-trifluoromethylphenyl)thiazole [preparation, for example, according to Sznaidman et al., Bioorg. Med. Chem. Lett. 13 (9), 1517-1522 (2003)].

¹H-NMR (400 MHz, DMSO-d₆): δ=1.36 (s, 6H), 2.34 (s, 3H), 3.68 (s, 4H), 3.76 (s, 2H), 6.34-6.36 (m, 1H), 6.43-6.45 (m, 1H), 7.42 (m, 4H), 7.67 (s, 1H), 7.84 (d, 2H), 8.11 (d, 2H).

MS (ESIpos): m/z=561 [M+H]⁺

HPLC (method 1): Rₜ=4.85 min

### Example 29

### 2-{[4-({(2-Furylmethyl)[(1-phenyl-1H-1,2,4-triazol-3-yl)methyl]amino}methyl)phenyl]thio}-2-methylpropionic acid

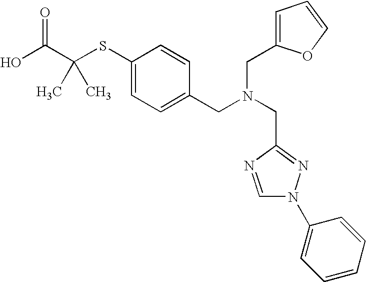

The title compound is prepared analogously to Example 19 starting with Example 19A.

¹H-NMR (400 MHz, DMSO-d₆): δ=1.35 (s, 6H), 3.70 (s, 2H), 3.721 (s, 2H), 3.733 (s, 2H), 6.37-6.39 (m, 1H), 6.40-6.43 (m, 1H), 7.37-7.45 (m, 5H), 7.55 (m, 2H), 7.62 (s, 1H), 7.85 (d, 2H), 12.57 (br. s, 1H).

MS (ESIpos): m/z=463 [M+H]⁺

HPLC (method 1): Rₜ=4.32 min

### Example 30

### 2-{[4-({(2-Furylmethyl)[(4-phenyl-1H-imidazol-2-yl)methyl]amino}methyl)phenyl]thio}-2-methylpropionic acid

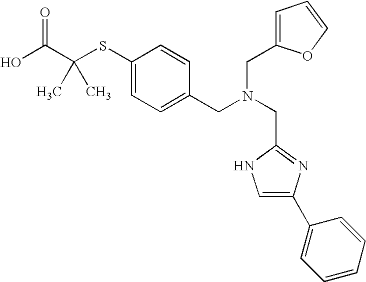

The title compound is prepared analogously to Example 19 starting with Example 21A.

¹H-NMR (400 MHz, DMSO-d₆): δ=1.30 (s, 6H), 3.71 (s, 2H), 3.76 (s, 2H), 4.06 (s, 2H), 6.38-6.43 (m, 2H), 7.36-7.46 (m, 5H), 7.50 (m, 2H), 7.53 (s, 1H), 7.78 (d, 2H), 7.99 (s, 1H), 12.62 (br. s, 1H), 14.32 (br. s, 1H).

MS (ESIpos): m/z=462 [M+H]⁺

HPLC (method 1): Rₜ=4.33 min

### Example 31

### 2-({4-[((2-Furylmethyl) {[3-(4-nitrophenyl)-1,2,4-oxadiazol-5-yl]methyl}amino)methyl]phenyl}-thio)-2-methylpropionic acid

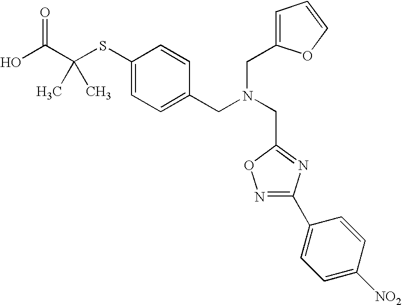

The title compound is prepared analogously to Example 2.

¹H-NMR (400 MHz, DMSO-d₆): δ=1.37 (s, 6H), 3.81 (s, 4H), 4.07 (s, 2H), 6.36-6.38 (m, 1H), 6.38-6.41 (m, 1H), 7.40 (m, 4H), 7.62 (s, 1H), 8.28 (d, 2H), 8.43 (d, 2H), 12.60 (br. s, 1H).

MS (ESIpos): m/z=509.5 [M+H]⁺

HPLC (method 1): Rₜ=5.04 min

### Example 32

### 2-({4-[({[3-(2,4-Dimethylphenyl)-1,2,4-oxadiazol-5-yl]methyl}amino)methyl]phenyl}thio)-2-methylpropionic acid

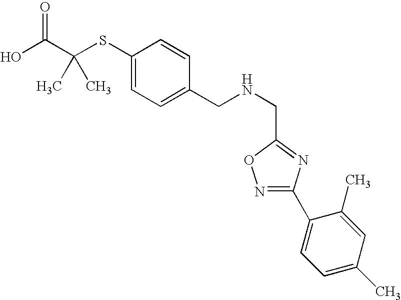

The title compound is prepared analogously to Example 2 starting with tert-butyl 2-{[4-(amino-methyl)phenyl]thio}-2-methylpropionate (Example 23A).

¹H-NMR (400 MHz, DMSO-d₆): δ=1.38 (s, 6H), 2.36 (s, 3H), 2.55 (s, 3H), 2.69 (s, 1H), 4.07 (s, 2H), 4.83 (s, 2H), 7.20 (d, 1H), 7.24 (s, 1H), 7.44 (s, 4H), 7.85 (d, 1H).

MS (ESIpos): m/z=412 [M+H]⁺

HPLC (method 1): Rₜ=4.41 min

### Example 33

### 2-[(4-{[{[3-(4-Chlorophenyl)isoxazol-5-yl]methyl}(2-furylmethyl)amino]methyl}phenyl)thio]-2-methylpropionic acid

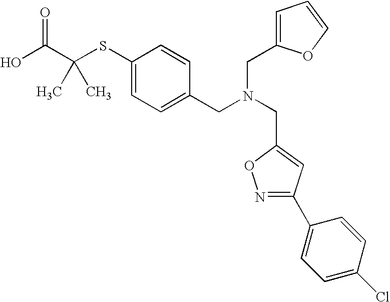

The title compound is prepared analogously to Example 3.

¹H-NMR (400 MHz, DMSO-d₆): δ=1.37 (s, 6H), 3.68 (s, 4H), 3.84 (s, 2H), 6.38-6.40 (m, 1H), 6.51-6.54 (m, 1H), 7.02 (s, 1H), 7.40 (m, 4H), 7.59 (d, 2H), 7.65 (s, 1H), 7.93 (d, 2H), 12.61 (br. s, 1H).

MS (ESIpos): m/z=497.5 [M+H]⁺

HPLC (method 1): Rₜ=4.71 min

### Example 34

### 2-[(4-{[{[3-(2,4-Difluorophenyl)isoxazol-5-yl]methyl}(2-furylmethyl)amino]methyl}phenyl)thio]-2-methylpropionic acid

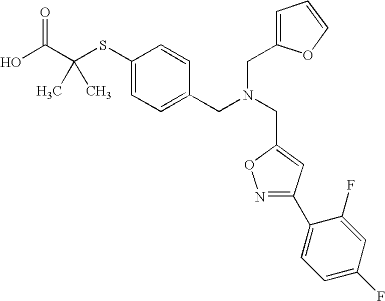

The title compound is prepared analogously to Example 3.

¹H-NMR (300 MHz, DMSO-d₆): δ=1.37 (s, 6H), 3.68 (s, 4H), 3.34 (s, 2H), 6.37-6.39 (m, 1H), 6.42-6.45 (m, 1H), 6.81 (d, 1H), 7.27 (ddd, 1H), 7.39 (m, 4H), 7.49 (ddd, 1H), 7.64 (d, 1H), 7.93-8.02 (m, 1H), 12.60 (br. s, 1H).

MS (ESIpos): m/z=499 [M+H]⁺

HPLC (method 1): Rₜ=4.58 min

### Example 35

### 2-[(4-{[{[3-(4-methoxyphenyl)isoxazol-5-yl]methyl}(2-furylmethyl)amino]methyl}phenyl)thio]-2-methylpropionic acid

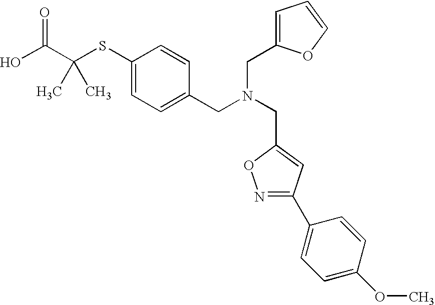

The title compound is prepared analogously to Example 3.

¹H-NMR (300 MHz, CDCl₃): δ=1.51 (s, 6H), 3.71 (s, 2H), 3.74 (s, 2H), 3.83 (s, 2H), 3.87 (s, 3H), 6.25-6.28 (m, 1H), 6.32-6.36 (m, 1H), 6.45 (s, 1H), 6.94-7.01 (m, 2H), 7.37-7.43 (m, 3H), 7.49 (d, 2H), 7.72-7.78 (m, 2H).

MS (ESIpos): m/z=493 [M+H]⁺

HPLC (method 1): Rₜ=4.50 min

The following compounds are prepared analogously to the examples described above from the corresponding starting materials:

### Example 36

### 2-[(4-{[{[3-(2,4-Dimethylphenyl)-1,2,4-oxadiazol-5-yl]methyl}(3-thienylmethyl)amino]methyl}-phenyl)thio]-2-methylpropionic acid

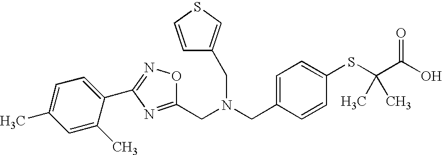

### Example 37

### 2-{[4-({(2-Furylmethyl)[(2-phenoxy-1,3-thiazol-5-yl)methyl]amino}methyl)phenyl]thio}-2-methylpropionic acid

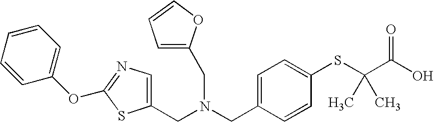

### Example 38

### 2-[(4-{[{[2-(2,4-Dimethylphenoxy)-1,3-thiazol-5-yl]methyl}(2-furylmethyl)amino]methyl}-phenyl)thio]-2-methylpropionic acid

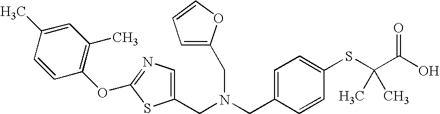

### Example 39

### 2-[(4-{[[(3-Benzoyl-1,2,4-oxadiazol-5-yl)methyl](2-furylmethyl)amino]methyl}phenyl)thio]-2-methylpropionic acid

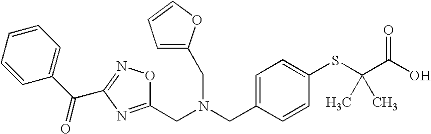

### Example 40

### 2-[(4-{[{[3-(2,4-Dimethylbenzoyl)-1,2,4-oxadiazol-5-yl]methyl}(2-furylmethyl)amino]methyl}-phenyl)thio]-2-methylpropionic acid

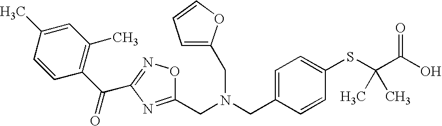

### Example 41

### 2-{[4-({{[3-(2,4-Dimethylphenyl)-1,2,4-oxadiazol-5-yl]methyl}[(4-methyl-1,3-oxazol-2-yl)-methyl]amino}methyl)phenyl]thio}-2-methylpropionic acid

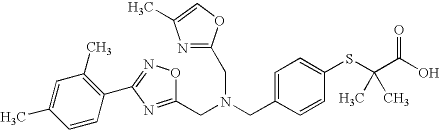

### Example 42

### 2-{[4-({(2-Furylmethyl)[[3-(pyridin-2-yl)-1,2,4-oxadiazol-5-yl]methyl]amino}methyl)phenyl]-thio}-2-methylpropionic acid

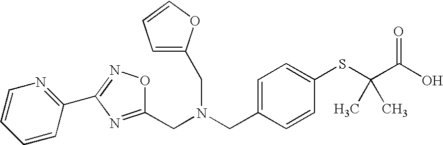

### Example 43

### 2-{[4-({(2-Furylmethyl)[[3-(pyridin-3-yl)-1,2,4-oxadiazol-5-yl]methyl]amino}methyl)phenyl]-thio}-2-methylpropionic acid

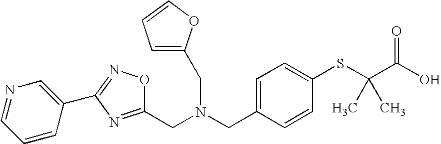

### Example 44

### 2-{[4-({(2-Furylmethyl)[[3-(pyridin-4-yl)-1,2,4-oxadiazol-5-yl]methyl]amino}methyl)phenyl]-thio}-2-methylpropionic acid

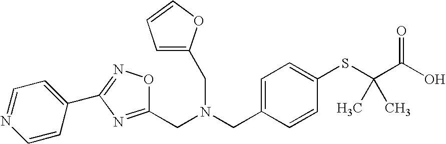

### Example 45

### 2-({4-[((2-methoxyethyl){[5-methyl-2-(3-methylbenzyl)-1,3-oxazol-4-yl]methyl}amino)methyl]-phenyl}thio)-2-methylpropionic acid

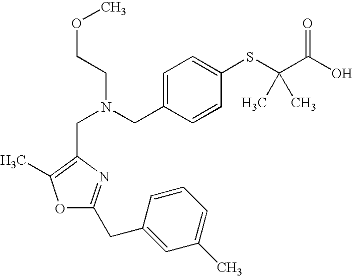

2.5 ml of trifluoroacetic acid are added to a solution of 0.45 g (0.83 mmol) of the compound from Example 93A in 5.0 ml of dichloromethane, and the mixture is stirred at room temperature for two hours. The reaction mixture is concentrated under reduced pressure and the residue is taken up in ethyl acetate. The organic phase is washed twice with water, once with 20% strength sodium acetate solution and once with saturated sodium chloride solution and dried over anhydrous magnesium sulphate. The solvent is removed under reduced pressure and the residue is purified over a Biotage cartridge 40S (mobile phase: dichloromethane/methanol 20:1). This gives 0.36 g (89% of theory) of the title compound as a yellowish resin.

LC/MS (method 5): Rₜ=1.91 min; MS (ESIpos): m/z=483 [M+H]⁺

¹H-NMR (400 MHz, CDCl₃): δ [ppm]=1.50 (s, 6H), 2.13 (s, 3H), 2.32 (s, 3H), 2.83 (t, 2H, J=5.9 Hz), 3.28 (s, 3H), 3.56 (t, 2H, J=5.9 Hz), 3.64 (s, 2H), 3.81 (s, 2H), 4.00 (s, 2H), 7.04-7.09 (m, 3H), 7.19 (m, 1H), 7.33 (d, 2H, J=8.5 Hz), 7.47 (d, 2H, J=8.5 Hz).

The following compounds are prepared analogously to Example 45 from the starting materials stated:

| | | | Yield | |
|---|---|---|---|---|
| | | Starting | [% of | |
| Example | Structure | material | theory] | LC/MS |
| 46 | 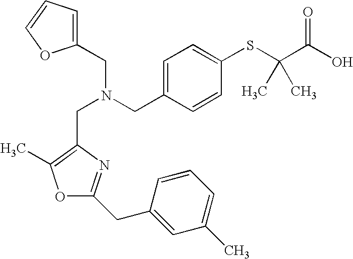 | Example 79A | 65% | Rₜ = 2.08 min; MS (ESIpos):m/z = 505 [M + H]⁺ (method 2) |
| | ¹H-NMR (400 MHz, CDCl₃): δ [ppm] = 1.50 (s, 6H), 2.15 (s, 3H), 2.31 (s, 3H), 3.50 (s, 2H), 3.65 (s, 2H), 3.71 (s, 2H), | | | |
| | 4.01 (s, 2H), 6.22 (m, 1H), 6.31 (m, 1H), 7.03-7.09 (m, 3H), 7.19 (m, 1H), 7.33 (d, 2H), 7.37 (m, 1H), 7.46 (d, 2H) | | | |
| 47 | 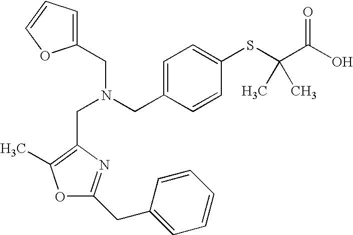 | Example 80A | 60% | Rₜ = 1.16 min; MS (ESIpos):m/z = 491 [M + H]⁺ (method 2) |
| | ¹H-NMR (400 MHz, CDCl₃): δ [ppm] = 1.49 (s, 6H), 2.15 (s, 3H), 3.50 (s, 2H), 3.64 (s, 2H), 3.70 (s, 2H), 4.05 (s, 2H), | | | |
| | 6.22 (m, 1H), 6.31 (m, 1H), 7.23-7.37 (m, 8H), 7.45 (d, 2H). | | | |
| 48 | 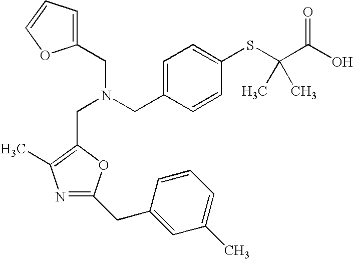 | Example 81A | 97% | Rₜ = 2.62 min; MS (ESIpos):m/z = 505 [M + H]⁺ (method 5) |
| | ¹H-NMR (400 MHz, CDCl₃): δ [ppm] = 1.52 (s, 6H), 1.96 (s, 3H), 2.31 (s, 3H), 3.57 (s, 2H), 3.58 (s, 2H), 3.67 (s, 2H), | | | |
| | 4.04 (s, 2H), 6.17 (m, 1H), 6.31 (m, 1H), 7.04-7.11 (m, 3H), 7.18-7.22 (m, 3H), 7.38 (m, 1H), 7.41 (d, 2H). | | | |
| 49 | 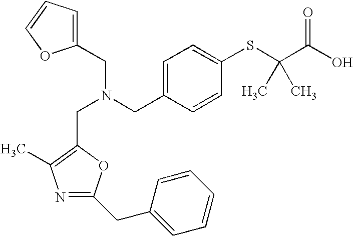 | Example 82A | 76% | Rₜ = 2.46 min; MS (ESIpos):m/z = 491 [M + H]⁺ (method 3) |
| | ¹H-NMR (400 MHz, CDCl₃): δ [ppm] = 1.53 (s, 6H), 1.94 (s, 3H), 3.56 (s, 2H), 3.58 (s, 2H), 3.68 (s, 2H), 4.08 (s, 2H), | | | |
| | 6.17 (m, 1H), 6.32 (m, 1H), 7.19 (d, 2H), 7.30 (m, 5H), 7.38 (m, 1H), 7.40 (d, 2H). | | | |
| 50 | 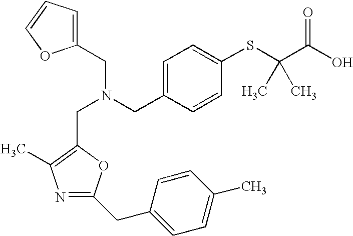 | Example 83A | 79% | Rₜ = 2.46 min; MS (ESIpos):m/z = 505 [M + H]⁺ (method 2) |
| | ¹H-NMR (400 MHz, CDCl₃): δ [ppm] = 1.53 (s, 6H), 1.94 (s, 3H), 2.31 (s, 3H), 3.57 (s, 2 × 2H), 3.68 (s, 2H), 4.04 (s, | | | |
| | 2H), 6.18 (m, 1H), 6.32 (m, 1H), 7.11 (d, 2H), 7.18-7.21 (m, 4H), 7.38 (m, 1H), 7.40 (d, 2H). | | | |
| 51 | 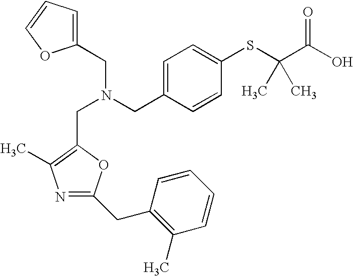 | Example 84A | 79% | Rₜ = 2.59 min; MS (ESIpos):m/z = 505 [M + H]⁺ (method 5) |
| | ¹H-NMR (400 MHz, CDCl₃): δ [ppm] = 1.53 (s, 6H), 1.94 (s, 3H), 2.34 (s, 3H), 3.56 (s, 2 × 2H), 3.67 (s, 2H), 4.08 (s, | | | |
| | 2H), 6.16 (m, 1H), 6.31 (m, 1H), 7.15-7.22 (m, 6H), 7.38 (m, 1H), 7.41 (d, 2H). | | | |
| 52 | 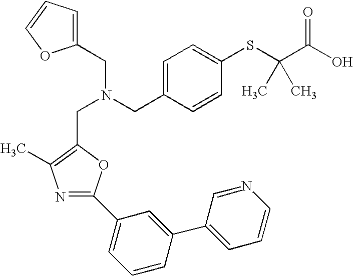 | Example 85A | 72% | Rₜ = 2.62 min; MS (ESIpos):m/z = 554 [M + H]⁺ (method 3) |
| | ¹H-NMR (400 MHz, CDCl₃): δ [ppm] = 1.56 (s, 6H), 2.27 (s, 3H), 3.69 (s, 2H), 3.73 (s, 2H), 3.74 (s, 2H), 6.24 (m, | | | |
| | 1H), 6.34 (m, 1H), 7.40 (m, 1H), 7.52-7.60 (m, 6H), 7.66 (d, 1H), 8.12 (d, 1H), 8.17 (d, 1H), 8.28 (s, 1H), 8.61 (m, 1H), | | | |
| | 9.22 (d, 1H). | | | |
| 53 | 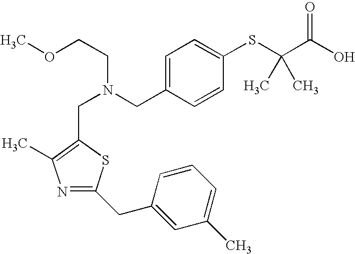 | Example 94A | 94% | Rₜ = 2.26 min; MS (ESIpos):m/z = 499 [M + H]⁺ (method 5) |
| | ¹H-NMR (400 MHz, CDCl₃): δ [ppm] = 1.50 (s, 6H), 2.26 (s, 3H), 2.33 (s, 3H), 2.66 (t, 2H), 3.26 (s, 3H), 3.44 (t, 2H), | | | |
| | 3.62 (s, 2H), 3.69 (s, 2H), 4.20 (s, 2H), 7.05-7.12 (m, 3H), 7.18-7.25 (m, 3H), 7.43 (d, 2H). | | | |
| 54 | 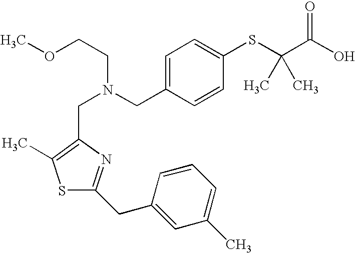 | Example 95A | 51% | Rₜ = 2.08 min; MS (ESIpos):m/z = 499 [M + H]⁺ (method 5) |
| | ¹H-NMR (400 MHz, CDCl₃): δ [ppm] = 1.51 (s, 6H), 2.26 (s, 3H), 2.32 (s, 3H), 2.89 (br. s, 2H), 3.30 (s, 3H), 3.61 (br. | | | |
| | s, 2H), 3.83 (br. s, 2 × 2H), 4.19 (s, 2H), 7.06-7.10 (m, 3H), 7.20 (m, 1H), 7.34-7.37 (m, 2H), 7.46 (d, 2H). | | | |
| 55 | 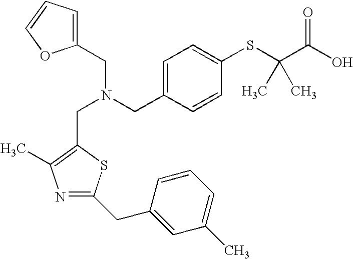 | Example 86A | 78% | Rₜ = 2.78 min; MS (ESIpos):m/z = 521 [M + H]⁺ (method 2) |
| | ¹H-NMR (400 MHz, CDCl₃): δ [ppm] = 1.51 (s, 6H), 2.25 (s, 3H), 2.33 (s, 3H), 3.58 (s, 2H), 3.63 (s, 2H), 3.64 (s, 2H), | | | |
| | 4.21 (s, 2H), 6.15 (d, 1H), 6.31 (m, 1H), 7.05-7.12 (m, 3H), 7.20 (d, 1H), 7.27 (d, 2H), 7.37 (d, 1H), 7.44 (d, 2H). | | | |
| 56 | 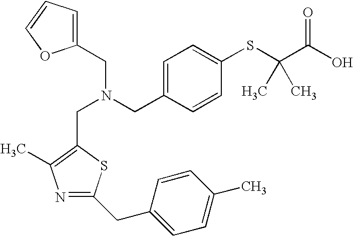 | Example 87A | 35% | Rₜ = 2.95 min; MS (ESIpos):m/z = 521 [M + H]⁺ (method 3) |
| | ¹H-NMR (400 MHz, CDCl₃): δ [ppm] = 1.51 (s, 6H), 2.25 (s, 3H), 2.33 (s, 3H), 3.57 (s, 2H), 3.61 (s, 2H), 3.62 (s, 2H), | | | |
| | 4.20 (s, 2H), 6.13 (d, 1H), 6.31 (m, 1H), 7.12 (d, 2H), 7.19 (d, 2H), 7.27 (d, 2H), 7.37 (s, 1H), 7.44 (d, 2H). | | | |
| 57 | 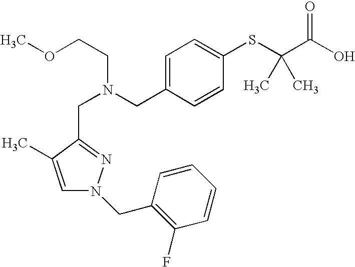 | Example 96A | 87% | Rₜ = 1.84 min; MS (ESIpos):m/z = 486 [M + H]⁺ (method 5) |
| | ¹H-NMR (400 MHz, CDCl₃): δ [ppm] = 1.50 (s, 6H), 2.00 (s, 3H), 2.79 (t, 2H), 3.26 (s, 3H), 3.52 (t, 2H), 3.72 (s, 2H), | | | |
| | 3.76 (s, 2H), 5.27 (s, 2H), 7.00-7.07 (m, 3H), 7.13 (s, 1H), 7.27 (m, 1H), 7.29 (d, 2H), 7.43 (d, 2H). | | | |
| 58 | 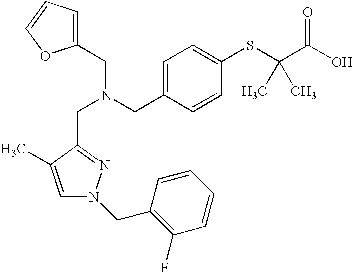 | Example 88A | 57% | Rₜ = 1.91 min; MS (ESIpos):m/z = 508 [M + H]⁺ (method 2) |
| | ¹H-NMR (400 MHz, CDCl₃): δ [ppm] = 1.50 (s, 6H), 2.00 (s, 3H), 3.62 (br. s, 3 × 2H), 5.27 (s, 2H), 6.24 (m, 1H), 6.32 | | | |
| | (m, 1H), 7.01-7.08 (m, 3H), 7.13 (s, 1H), 7.25 (m, 1H), 7.32-7.38 (m, 3H), 7.44 (d, 2H). | | | |
| 59 | 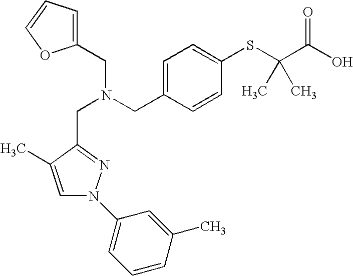 | Example 89A | 92% | Rₜ = 2.36 min; MS (ESIpos):m/z = 490 [M + H]⁺ (method 3) |
| | ¹H-NMR (400 MHz, CDCl₃): δ [ppm] = 1.48 (s, 6H), 2.04 (s, 3H), 2.39 (s, 3H), 3.65 (s, 2H), 3.69 (br. s, 2 × 2H), 6.26 | | | |
| | (m, 1H), 6.32 (m, 1H), 7.03 (d, 1H), 7.28 (m, 1H), 7.33-7.39 (m, 3H), 7.43-7.46 (m, 4H), 7.60 (s, 1H). | | | |
| 60 | 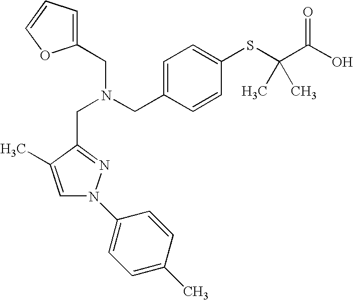 | Example 90A | 81% | Rₜ = 2.37 min; MS (ESIpos):m/z = 490 [M + H]⁺ (method 5) |
| | ¹H-NMR (400 MHz, CDCl₃): δ [ppm] = 1.49 (s, 6H), 2.04 (s, 3H), 2.36 (s, 3H), 3.66 (s, 2H), 3.69 (br. s, 2 × 2H), 6.27 | | | |
| | (m, 1H), 6.33 (m, 1H), 7.20 (d, 2H), 7.35-7.39 (m, 3H), 7.43 (s, 1H), 7.46-7.50 (m, 3H), 7.57 (s, 1H). | | | |
| 61 | 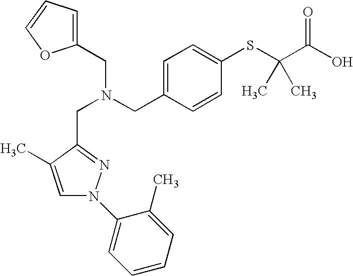 | Example 91A | 78% | Rₜ = 2.07 min; MS (ESIpos):m/z = 490 [M + H]⁺ (method 2) |

¹H-NMR (400 MHz, CDCl₃): δ [ppm] = 1.49 (s, 6H), 2.05 (s, 3H), 2.24 (s, 3H), 3.68 (s, 2H), 3.70 (br. s, 2 × 2H), 6.28
(m, 1H), 6.32 (m, 1H), 7.26-7.30 (m, 5H), 7.36-7.39 (m, 3H), 7.46 (d, 2H).

### Example 62

### 2-[(4-{[{[3-(4-Fluorobenzyl)-1,2,4-oxadiazol-5-yl]methyl}(2-furylmethyl)amino]methyl}phenyl)-thio]-2-methylpropanoic acid hydrochloride

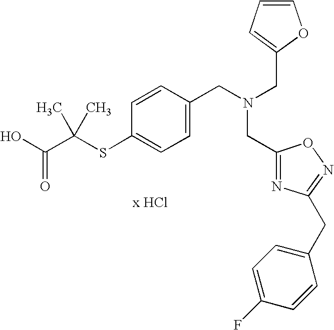

10 ml of 4 M hydrogen chloride in dioxane are added to 254 mg (0.46 mmol) of the compound from Example 101A, and the mixture is stirred at RT overnight. The solvent is distilled off under reduced pressure. This gives 240 mg (98% of theory) of the title compound.

LC/MS (method 5): Rₜ=2.84 min; MS (ESIpos): m/z=496 [M+H]⁺

¹H-NMR (400 MHz, DMSO-d₆): δ [ppm]=1.37 (s, 6H), 3.74 (s, 4H), 3.94 (s, 2H), 4.09 (s, 2H), 6.30 (d, 1H), 6.39 (m, 1H), 7.16 (t, 2H), 7.30-7.42 (m, 6H), 7.60 (m, 1H), 12.60 (br. s, 1H).

### Example 63

### 2-{[4-({(2-Furylmethyl)[(2-{[3-(trifluoromethyl)phenyl]amino}-1,3-thiazol-4-yl)methyl]amino}-methyl)phenyl]thio}-2-methylpropanoic acid hydrochloride

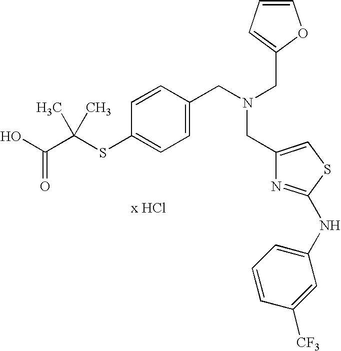

10 ml of a 4 M solution of hydrogen chloride in dioxane are added to 120 mg (0.19 mmol) of the compound from Example 103A, and the mixture is stirred at RT overnight. The solvent is distilled off under reduced pressure. This gives 100 mg (82% of theory) of the title compound.

LC/MS (method 2): Rₜ=2.08 min; MS (ESIpos): m/z=562 [M+H]⁺.

The free base is obtained by purification by preparative HPLC in a yield of 60% (mobile phase: acetonitrile/water with 0.1% formic acid, gradient 20:80→95:5):

¹H-NMR (400 MHz, DMSO-d₆): δ [ppm]=1.36 (s, 6H), 3.58 (s, 2H), 3.69 (s, 2H), 3.70 (s, 2H), 6.35 (d, 1H), 6.41 (dd, 1H), 6.77 (s, 1H), 7.26 (d, 1H), 7.40 (s, 4H), 7.53 (t, 1H), 7.61 (d, 1H), 7.75 (d, 1H), 8.29 (s, 1H), 10.52 (s, 1H), 12.57 (br. s, 1H).

### Example 64

### 2-(4-{[[2-(3-Chlorophenoxy)thiazol-5-yl-methyl](2-methoxyethyl)amino]methyl}phenylthio)-2-methylpropionic acid hydrochloride

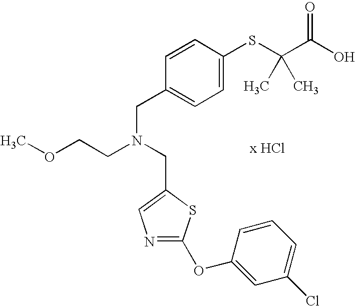

47 mg (0.08 mmol) of the compound from Example 107A and 5 ml of 4 M hydrogen chloride in dioxane are stirred at room temperature for eight hours. All volatile components are removed under reduced pressure. This gives 43 mg (99% of theory) of the title compound.

LC/MS (method 3): Rₜ=2.36 min; MS (ESIpos): m/z=507 [M+H]⁺

¹H-NMR (400 MHz, DMSO-d₆): δ [ppm]=1.4 (s, 6H), 2.6 (t, 2H), 3.2 (s, 3H), 3.4 (t, 2H), 3.65 (s, 2H), 3.75 (s, 2H), 7.2 (s, 1H), 7.35 (d, 2H), 7.4 (m, 3H), 7.5 (m, 3H), 12.6 (s, 1H).

### Example 65

### 2-({4-[((2-methoxyethyl) {[1-(3-methylbenzyl)-1H-1,2,3-triazol-4-yl]methyl}amino)methyl]-phenyl}thio)-2-methylpropionic acid

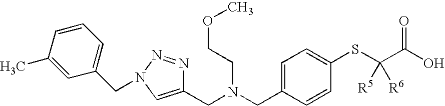

3.0 ml of trifluoroacetic acid are added to a solution of 0.48 g (0.92 mmol) of the compound from Example 98A in 6.0 ml of dichloromethane, and the mixture is stirred at room temperature for two hours. The reaction mixture is concentrated under reduced pressure and the residue is taken up in ethyl acetate. The organic phase is washed twice with water, once with 20% strength sodium acetate solution and once with saturated sodium chloride solution and dried over anhydrous magnesium sulphate. The solvent is removed under reduced pressure and the residue is purified by preparative HPLC. This gives 0.35 g (80% of theory) of the title compound as a colourless resin.

LC/MS (method 3): Rₜ=1.70 min; MS (ESIpos): m/z=469 [M+H]⁺

¹H-NMR (400 MHz, DMSO-d₆): δ [ppm]=1.47 (s, 6H), 2.32 (s, 3H), 2.78 (t, 2H), 3.26 (s, 3H), 3.55 (t, 2H), 3.81 (s, 2H), 3.96 (s, 2H), 5.46 (s, 2H), 7.05 (d, 2H), 7.14 (d, 1H), 7.23 (d, 1H), 7.31 (d, 2H), 7.44 (d, 2H), 7.50 (s, 1H).

### B. ASSESSMENT OF THE PHARMACOLOGICAL ACTIVITY

The pharmacological activity of the compounds according to the invention can be demonstrated by the following assays:

### 1. Cellular Transactivation Assay:

### a) Test Principle:

A cellular assay is used to identify activators of the peroxisome proliferator-activated receptor alpha (PPAR-alpha).

Since mammalian cells contain different endogenous nuclear receptors which may complicate an unambiguous interpretation of the results, an established chimera system is used in which the ligand binding domain of the human PPARα receptor is fused to the DNA binding domain of the yeast transcription factor GAL4. The resulting GAL4-PPARα chimera is co-transfected and stably expressed in CHO cells having a reporter construct.

### b) Cloning:

The GAL4-PPARα expression construct contains the ligand binding domain of PPARα (amino acids 167-468) which is PCR-amplified and cloned into the vector pcDNA3.1. This vector already contains the GAL4 DNA binding domain (amino acids 1-147) of the vector pFC2-dbd (Stratagene). The reporter construct, which contains five copies of the GAL4 binding site upstream of a thymidine kinase promoter, expresses firefly luciferase (Photinus pyralis) following activation and binding of GAL4-PPARα.

### c) Transactivation Assay (Luciferase Reporter):

CHO (Chinese hamster ovary) cells are sown in DMEM/F12 medium (BioWhittaker) supplemented by 10% foetal calf serum and 1% penicillin/streptomycin (GIBCO), at a cell density of 2×10³ cells per well in a 384-well plate (Greiner). The cells are cultivated at 37° C. for 48 h and then stimulated. To this end, the substances to be tested are taken up in CHO-A-SFM medium (GIBCO) supplemented by 10% foetal calf serum and 1% penicillin/streptomycin (GIBCO) and added to the cells. After a stimulation period of 24 hours, the luciferase activity is measured using a video camera. The relative light units measured give, as a function of the substance concentration, a sigmoidal stimulation curve. The EC₅₀ values are calculated using the computer programme GraphPad PRISM (Version 3.02).

In this test, the compounds according to the invention show EC₅₀ values of from 5 μM to 1 nM.

### 2. Fibrinogen Determination:

To determine the effect on the plasma fibrinogen concentration, male Wistar rats or NMRI mice are treated with the substance to be examined by stomach tube administration or by addition to the feed for a period of 4-9 days. Under terminal anaesthesia, citrate blood is then obtained by heart puncture. The plasma fibrinogen concentrations are determined according to the Clauss method [A. Clauss, Acta Haematol. 17, 237-46 (1957)] by measuring the thrombin time using human fibrinogen as standard.

3. Description of a Test for Finding Pharmacologically Active Substances which Increase Apoprotein A1 (ApoA1) and HDL Cholesterol (HDL-C) Concentrations in the Serum of Transgenic Mice Transfected with the Human ApoA1 Gene (Hapoa1) and/or Lower Serum Triglycerides (TG):

The substances to be examined in vivo for their HDL-C-increasing activity are administered orally to male transgenic hApoA1 mice. One day prior to the start of the experiment, the animals are randomized into groups with the same number of animals, generally n=7-10. Throughout the experiment, the animals have drinking water and feed ad libitum. The substances are administered orally once a day for 7 days. To this end, the test substances are dissolved in a solution of Solutol HS 15+ethanol+saline (0.9%) in a ratio of 1+1+8 or in a solution of Solutol HS 15+saline (0.9%) in a ratio of 2+8. The dissolved substances are administered in a volume of 10 ml/kg of body weight using a stomach tube. Animals which have been treated in exactly the same manner but have only been given the solvent (10 ml/kg of body weight), without test substance, serve as control group.

Prior to the first administration of substance, a blood sample from each of the mice is taken by puncture of the retroorbital venous plexus, to determine ApoA1, serum cholesterol, HDL-C and serum triglycerides (TG) (zero value). Subsequently, using a stomach tube, the test substance is administered for the first time to the animals. 24 hours after the final administration of substance (on the 8^{th} day after the beginning of treatment), a blood sample from each of the animals is again taken by puncture of the retroorbital venous plexus, to determine the same parameters. The blood samples are centrifuged and, after the serum has been obtained, TG, cholesterol, HDL-C and human ApoA1 are determined using a Cobas Integra 400 plus instrument (Cobas Integra, Roche Diagnostics GmbH, Mannheim, Germany) using the respective cassettes (TRIGL, CHOL2, HDL-C and APOAT). HDL-C is determined by gel filtration and post-column derivatization with MEGA cholesterol reagent (Merck KGaA) analogously to the method of Garber et al. [J. Lipid Res. 41, 1020-1026 (2000)].

The effect of the test substances on HDL-C, hApoA1 and TG concentrations is determined by subtracting the value measured for the first blood sample (zero value) from the value measured for the second blood sample (after the treatment). The means of the differences of all HDL-C, hApoA1 and TG values of a group are determined and compared with the mean of the differences of the control group. Statistical evaluation is carried out using Student's t-Test, after the variances have been checked for homogeneity.

Substances which increase the HDL-C of the treated animals, compared to that of the control group, in a statistically significant manner (p<0.05) by at least 20% or which lower TG in a statistically significant manner (p<0.05) by at least 25% are considered to be pharmacologically effective.

### C. WORKING EXAMPLES OF PHARMACEUTICAL COMPOSITIONS

The compounds according to the invention can be converted into pharmaceutical preparations in the following ways:

### Tablet:

### Composition:

100 mg of the compound according to the invention, 50 mg of lactose (monohydrate), 50 mg of maize starch (native), 10 mg of polyvinylpyrrolidone (PVP 25) (from BASF, Ludwigshafen, Germany) and 2 mg of magnesium stearate.

Tablet weight 212 mg. Diameter 8 mm, radius of curvature 12 mm.

### Production:

The mixture of compound according to the invention, lactose and starch is granulated with a 5% strength solution (n/m) of the PVP in water. The granules are dried and then mixed with the magnesium stearate for 5 minutes. This mixture is compressed using a conventional tablet press (see above for the dimensions of the tablet). A compressive force of 15 kN is used as a guideline for the compression.

Suspension which can be Administered Orally:

### Composition:

1000 mg of the compound according to the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound according to the invention.

### Production:

The Rhodigel is suspended in ethanol, and the compound according to the invention is added to the suspension. The water is added while stirring. The mixture is stirred for about 6 h until the swelling of the Rhodigel is complete.

Solution which can be Administered Orally:

### Composition:

500 mg of the compound according to the invention, 2.5 g of polysorbate and 97 g of polyethylene glycol 400.20 g of oral solution correspond to a single dose of 100 mg of the compound according to the invention.

### Production:

The compound according to the invention is suspended in the mixture of polyethylene glycol and polysorbate with stirring. Stirring is continued until the compound according to the invention has dissolved completely.

i.v. Solution:

The compound according to the invention is, at a concentration below saturation solubility, dissolved in a physiologically acceptable solvent (for example isotonic saline, glucose solution 5% and/or PEG 400 solution 30%). The solution is subjected to sterile filtration and filled into sterile and pyrogen-free injection containers.

1. Compound of the formula (I) 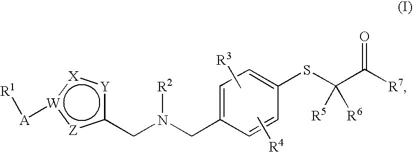
in which
W, X, Y and Z together with the carbon atom to which Y and Z are attached form a 5-membered heteroaryl ring which may optionally be mono- or disubstituted by identical or different substituents from the group consisting of (C₁-C₆)-alkyl and trifluoromethyl and in which
W represents C or N
and
X, Y and Z each represent C, N, O or S,
where at least one of the ring members W, X, Y and Z represents a heteroatom from the group consisting of N, O and S,
A, in the case that W represents C, represents a bond or represents CH₂, C(CH₃)₂, C(═O), O, S or NR⁸, in which
R⁸ represents hydrogen or (C₁-C₆)-alkyl,
and,
in the case that W represents N, represents a bond or represents CH₂ or C(═O),
R¹ represents (C₆-C₁₀)-aryl or 5- to 10-membered heteroaryl which may each be substituted up to four times by identical or different substituents selected from the group consisting of halogen, nitro, cyano, (C₁-C₆)-alkyl, (C₃-C₈)-cycloalkyl, phenyl, pyridyl, hydroxyl, (C₁-C₆)-alkoxy, trifluoromethyl, trifluoromethoxy, amino, mono- and di-(C₁-C₆)-alkylamino, R⁹—C(O)—NH—, R¹⁰—C(O)—, R¹¹R¹²N—C(O)—NH— and R¹³R¹⁴N—C(O)—, in which
R⁹ represents hydrogen, (C₁-C₆)-alkyl, (C₃-C₈)-cycloalkyl, phenyl or (C₁-C₆)-alkoxy,
R¹⁰ represents hydrogen, (C₁-C₆)-alkyl, (C₃-C₈)-cycloalkyl, phenyl, hydroxyl or (C₁-C₆)-alkoxy
and
R¹¹, R¹², R¹³ and R¹⁴ are identical or different and independently of one another represent hydrogen, (C₁-C₆)-alkyl, (C₃-C₈)-cycloalkyl or phenyl,
R² represents hydrogen, (C₆-C₁₀)-aryl, (C₁-C₆)-alkyl, (C₂-C₆)-alkenyl or (C₂-C₆)-alkynyl, in which alkyl, alkenyl and alkynyl may each be substituted by trifluoromethyl, fluorine, cyano, (C₁-C₆)-alkoxy, trifluoromethoxy, (C₆-C₁₀)-aryl or 5- or 6-membered heteroaryl, where all aryl and heteroaryl groups mentioned for their part may in each case be substituted up to three times by identical or different substituents selected from the group consisting of halogen, nitro, cyano, (C₁-C₆)-alkyl, hydroxyl, (C₁-C₆)-alkoxy, trifluoromethyl and trifluoromethoxy,
R³ and R⁴ are identical or different and independently of one another represent hydrogen, (C₁-C₆)-alkyl, (C₂-C₆)-alkenyl, (C₁-C₆)-alkoxy, trifluoromethyl, trifluoromethoxy or halogen,
R⁵ and R⁶ are identical or different and independently of one another represent hydrogen, (C₁-C₆)-alkyl, (C₁-C₆)-alkoxy or phenoxy or together with the carbon atom to which they are attached form a (C₃-C₈)-cycloalkyl ring,
and
R⁷ represents a group of the formula —NHR¹⁵ or —OR¹⁶, in which
R¹⁵ represents hydrogen, (C₁-C₆)-alkyl or (C₁-C₆)-alkylsulphonyl
and
R¹⁶ represents hydrogen or represents a hydrolyzable group which can be converted into the corresponding carboxylic acid,
and its salts, solvates and solvates of the salts.

2. Compound of the formula (I) according to claim 1, in which
W, X, Y and Z together with the carbon atom to which Y and Z are attached form a 5-membered heteroaryl ring which may optionally be mono- or disubstituted by identical or different substituents from the group consisting of (C₁-C₆)-alkyl and trifluoromethyl and in which
W represents C or N
and
X, Y and Z each represent C, N, O or S,
where at least one of the ring members W, X, Y and Z represents a heteroatom from the group consisting of N, O and S,
A, in the case that W represents C, represents a bond or represents CH₂, C(═O), O, S or NR⁸, in which
R⁸ represents hydrogen or (C₁-C₆)-alkyl,
and
in the case that W represents N, represents a bond or represents CH₂ or C(═O),
R¹ represents (C₆-C₁₀)-aryl or 5- to 10-membered heteroaryl which may in each case be substituted up to four times by identical or different substituents selected from the group consisting of halogen, nitro, cyano, (C₁-C₆)-alkyl, (C₃-C₈)-cycloalkyl, phenyl, hydroxyl, (C₁-C₆)-alkoxy, trifluoromethyl, trifluoromethoxy, amino, mono- and di-(C₁-C₆)-alkylamino, R⁹—C(O)—NH—, R¹⁰—C(O)—, R¹¹R¹²N—C(O)—NH— and R¹³R¹⁴N—C(O)—, in which
R⁹ represents hydrogen, (C₁-C₆)-alkyl, (C₃-C₈)-cycloalkyl, phenyl or (C₁-C₆)-alkoxy,
R¹⁰ represents hydrogen, (C₁-C₆)-alkyl, (C₃-C₈)-cycloalkyl, phenyl, hydroxyl or (C₁-C₆)-alkoxy
and
R¹¹, R¹², R¹³ and R¹⁴ are identical or different and independently of one another represent hydrogen, (C₁-C₆)-alkyl, (C₃-C₈)-cycloalkyl or phenyl,
R² represents hydrogen, (C₆-C₁₀)-aryl, (C₁-C₆)-alkyl, (C₂-C₆)-alkenyl or (C₂-C₆)-alkynyl, in which alkyl, alkenyl and alkynyl may in each case be substituted by trifluoromethyl, fluorine, cyano, (C₁-C₆)-alkoxy, trifluoromethoxy, (C₆-C₁₀)-aryl or 5- or 6-membered heteroaryl, where all aryl and heteroaryl groups mentioned for their part may in each case be substituted up to three times by identical or different substituents selected from the group consisting of halogen, nitro, cyano, (C₁-C₆)-alkyl, hydroxyl, (C₁-C₆)-alkoxy, trifluoromethyl and trifluoromethoxy,
R³ and R⁴ are identical or different and independently of one another represent hydrogen, (C₁-C₆)-alkyl, (C₂-C₆)-alkenyl, (C₁-C₆)-alkoxy, trifluoromethyl, trifluoromethoxy or halogen,
R⁵ and R⁶ are identical or different and independently of one another represent hydrogen, (C₁-C₆)-alkyl, (C₁-C₆)-alkoxy or phenoxy or together with the carbon atom to which they are attached form a (C₃-C₈)-cycloalkyl ring,
and
R⁷ represents a group of the formula —NHR¹⁵ or —OR⁶, in which
R¹⁵ represents hydrogen or (C₁-C₆)-alkyl
and
R¹⁶ represents hydrogen or represents a hydrolyzable group which can be converted into the corresponding carboxylic acid,
and its salts, solvates and solvates of the salts.

3. Compound of the formula (I) according to claim 1 or 2, in which
W, X, Y and Z together with the carbon atom to which Y and Z are attached form a 5-membered heteroaryl ring which may optionally be mono- or disubstituted by identical or different substituents from the group consisting of (C₁-C₄)-alkyl and trifluoromethyl and in which
W represents C or N
and
X, Y and Z each represent C, N, O or S,
where at least one of the ring members W, X, Y and Z represents N and at least one further of the ring members W, X, Y and Z represents a heteroatom from the group consisting of N, O and S,
A, in the case that W represents C, represents a bond or represents CH₂, C(═O), O, S or NR⁸, in which
R⁸ represents hydrogen or (C₁-C₄)-alkyl,
and,
in the case that W represents N, represents a bond or represents CH₂ or C(═O),
R¹ represents phenyl or 5- or 6-membered heteroaryl which may in each case be substituted up to four times by identical or different substituents selected from the group consisting of halogen, nitro, cyano, (C₁-C₄)-alkyl, (C₃-C₆)-cycloalkyl, phenyl, hydroxyl, (C₁-C₄)-alkoxy, trifluoromethyl, trifluoromethoxy, amino, mono- and di-(C₁-C₄)-alkylamino, R⁹—C(O)—NH—, R¹⁰—C(O)—, R¹¹R¹²N—C(O)—NH— and R¹³R¹⁴N—C(O)—, in which
R⁹ represents hydrogen, (C₁-C₄)-alkyl, (C₃-C₆)-cycloalkyl, phenyl or (C₁-C₄)-alkoxy,
R¹⁰ represents hydrogen, (C₁-C₄)-alkyl, (C₃-C₆)-cycloalkyl, phenyl, hydroxyl or (C₁-C₄)-alkoxy
and
R¹¹, R¹², R¹³ and R¹⁴ are identical or different and independently of one another represent hydrogen, (C₁-C₄)-alkyl, (C₃-C₆)-cycloalkyl or phenyl,
R² represents hydrogen, phenyl, (C₁-C₄)-alkyl, (C₂-C₄)-alkenyl or (C₂-C₄)-alkynyl, in which alkyl, alkenyl and alkynyl may in each case be substituted by trifluoromethyl, fluorine, cyano, (C₁-C₄)-alkoxy, phenyl or 5- or 6-membered heteroaryl, where all phenyl and heteroaryl groups mentioned for their part may in each case be substituted up to three times by identical or different substituents selected from the group consisting of halogen, nitro, cyano, (C₁-C₄)-alkyl, hydroxyl, (C₁-C₄)-alkoxy, trifluoromethyl and trifluoromethoxy,
R³ and R⁴ are identical or different and independently of one another represent hydrogen, (C₁-C₄)-alkyl, (C₁-C₄)-alkoxy, trifluoromethyl, trifluoromethoxy or halogen,
R⁵ and R⁶ are identical or different and independently of one another represent hydrogen, methyl, ethyl, methoxy, ethoxy or phenoxy or together with the carbon atom to which they are attached form a (C₃-C₆)-cycloalkyl ring,
and
R⁷ represents a group of the formula —NHR¹⁵ or —OR¹⁶, in which
R¹⁵ represents hydrogen or (C₁-C₄)-alkyl
and
R¹⁶ represents hydrogen or represents a hydrolyzable group which can be converted into the corresponding carboxylic acid,
and its salts, solvates and solvates of the salts.

4. Compound of the formula (I) according to claim 1, 2 or 3, in which
W, X, Y and Z together with the carbon atom to which Y and Z are attached form a 5-membered heteroaryl ring of the formula 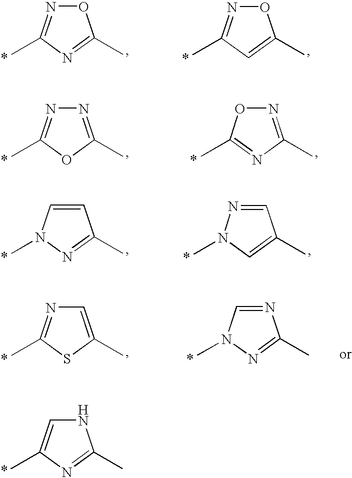
which may optionally be mono- or disubstituted by identical or different substituents from the group consisting of methyl and trifluoromethyl and in which * denotes the point of attachment to the group R¹-A-,
A, in the case that W represents C, represents a bond or represents CH₂, C(═O) or O
and,
in the case that W represents N, represents a bond or represents CH₂,
R¹ represents phenyl or pyridyl which may in each case be mono- or disubstituted by identical or different substituents selected from the group consisting of fluorine, chlorine, nitro, methyl, methoxy, trifluoromethyl and trifluoromethoxy,
R² represents hydrogen, propargyl or represents (C₁-C₄)-alkyl which may be substituted by fluorine, cyano, (C₁-C₄)-alkoxy, phenyl, furyl, thienyl, oxazolyl or thiazolyl, where phenyl and all heteroaromatic rings mentioned for their part may in each case be mono- or disubstituted by identical or different substituents selected from the group consisting of fluorine, chlorine, methyl, methoxy, trifluoromethyl and trifluoromethoxy,
R³ and R⁴ are identical or different and independently of one another represent hydrogen, methyl, methoxy, fluorine or chlorine,
R⁵ and R⁶ are identical or different and represent hydrogen or methyl,
and
R⁷ represents —OH, —NH₂ or —NHCH₃,
and its salts, solvates and solvates of the salts.

5. Compound of the formula (I) according to claim 1, 2 or 3, in which
W, X, Y and Z together with the carbon atom to which Y and Z are attached form a 5-membered heteroaryl ring of the formula 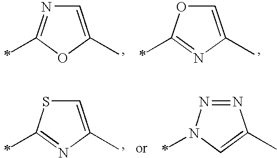
which may optionally be mono- or disubstituted by identical or different substituents from the group consisting of methyl and trifluoromethyl and in which * denotes the point of attachment to the group R¹-A-,
A, in the case that W represents C, represents a bond, CH₂ or O
and,
in the case that W represents N, represents a bond or represents CH₂,
R¹ represents phenyl which may be mono- or disubstituted by identical or different substituents selected from the group consisting of fluorine, chlorine, nitro, methyl, methoxy, trifluoromethyl and trifluoromethoxy,
R² represents (C₁-C₄)-alkyl, (C₂-C₄)-alkenyl or (C₂-C₄)-alkynyl which may in each case be substituted by fluorine, cyano, (C₁-C₄)-alkoxy, phenyl, furyl, thienyl, oxazolyl or thiazolyl, where phenyl and all heteroaromatic rings mentioned for their part may in each case be mono- or disubstituted by identical or different substituents selected from the group consisting of fluorine, chlorine, methyl, methoxy, trifluoromethyl and trifluoromethoxy,
R³ and R⁴ are identical or different and independently of one another represent hydrogen, methyl, methoxy, fluorine or chlorine,
R⁵ and R⁶ are identical or different and represent hydrogen or methyl, and
R⁷ represents —OH, —NH₂ or —NHCH₃,
and its salts, solvates and solvates of the salts.

6. Compound of the formula (I-A) 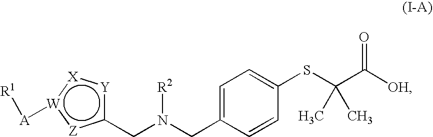
in which
A, W, X, Y, Z, R¹ and R² are in each case as defined in claims 1 to 5.

7. Process for preparing a compound of the formula (I) or (I-A) as defined in claims 1 to 6, characterized in that compounds of the formula (II) 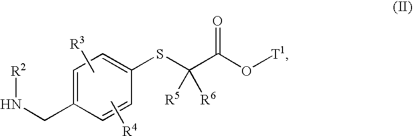
in which R², R³, R⁴, R⁵ and R⁶ are each as defined in claims 1 to 6
and
T¹ represents (C₁-C₄)-alkyl, preferably tert-butyl, or represents benzyl,
are initially reacted, in an inert solvent in the presence of a base, with a compound of the formula (III) 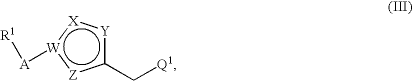
in which A, W, X, Y, Z and R¹ are each as defined in claims 1 to 6
and
Q¹ represents a suitable leaving group, such as, for example, halogen, mesylate, tosylate or triflate,
to give compounds of the formula (I-B) 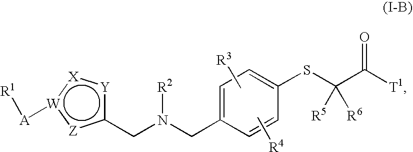
in which A, W, X, Y, Z, T¹, R¹, R², R³, R⁴, R⁵ and R⁶ are each as defined above, these are then converted, by basic or acidic hydrolysis or, in the case that T¹ represents benzyl, also hydrogenolytically, into carboxylic acids of the formula (I-C) 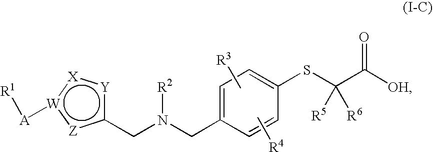
in which A, W, X, Y, Z, R¹, R², R³, R⁴, R⁵ and R⁶ are each as defined above,
and, if appropriate, subsequently converted by esterification or amidation methods known from the literature into the compounds of the formula (I)
and the compounds of the formula (I) are, if appropriate, using the appropriate (i) solvents and/or (ii) bases or acids, converted into their solvates, salts and/or solvates of the salts.

8. Process for preparing a compound of the formula (I-D) 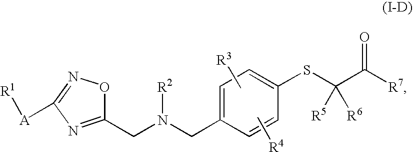
in which A, R¹, R², R³, R⁴, R⁵, R⁶ and R⁷ are each as defined in claims 1 to 6, characterized in that compounds of the formula (II) 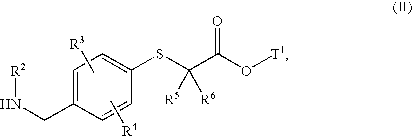
in which R², R³, R⁴, R⁵ and R⁶ are each as defined in claims 1 to 6
and
T¹ represents (C₁-C₄)-alkyl, preferably tert-butyl, or represents benzyl,
are initially, in an inert solvent, in the presence of a base, converted with a compound of the formula (IV) 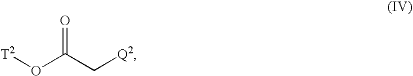
in which
T² represents (C₁-C₄)-alkyl, preferably methyl or ethyl,
and
Q² represents a suitable leaving group, such as, for example, halogen, mesylate, tosylate or triflate,
into compounds of the formula (V) 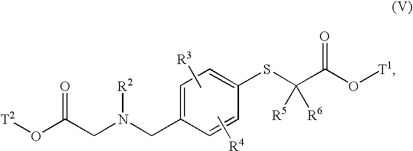
in which T¹, T², R², R³, R⁴, R⁵ and R⁶ are each as defined above,
subsequently, under suitable reaction conditions, hydrolyzed selectively to carboxylic acids of the formula (VI) 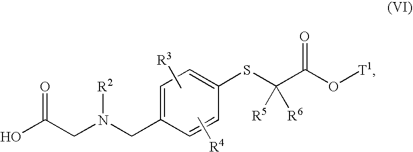
in which T¹, R², R³, R⁴, R⁵ and R⁶ are each as defined above,
then, in an inert solvent in the presence of a condensing agent, converted with a compound of the formula (VII) 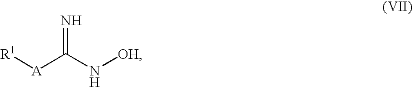
in which A and R¹ are each as defined in claims 1 to 6,
into compounds of the formula (VII) 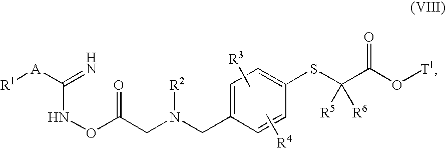
in which A, T¹, R¹, R², R³, R⁴, R⁵ and R⁶ are each as defined above,
then, with or without intermediate isolation, these are cyclized in the presence of a base to compounds of the formula (I-E) 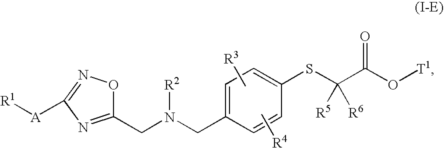
in which A, T¹, R¹, R², R³, R⁴, R⁵ and R⁶ are each as defined above,
then, by basic or acidic hydrolysis or, in the case that T¹ represents benzyl, also hydrogenolytically, converted into carboxylic acids of the formula (I-F) 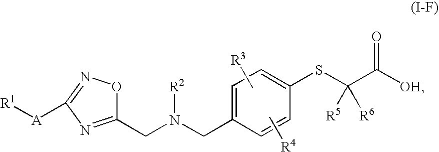
in which A, R¹, R², R³, R⁴, R⁵ and R⁶ are each as defined above,
and, if appropriate, finally converted by esterification or amidation methods known from the literature into the compounds of the formula (I-D).

9. Process for preparing a compound of the formula (I-G) 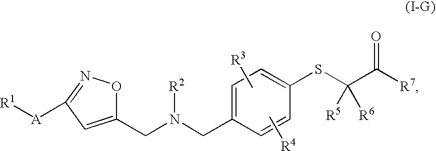
in which A represents a bond and R¹, R², R³, R⁴, R⁵, R⁶ and R⁷ are each as defined in claims 1 to 6, characterized in that compounds of the formula (II) 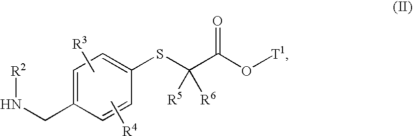
in which R², R³, R⁴, R⁵ and R⁶ are each as defined in claims 1 to 6
and
T¹ represents (C₁-C₄)-alkyl, preferably tert-butyl, or represents benzyl,
are initially, in an inert solvent, in the presence of a base, converted with a compound of the formula (IX) 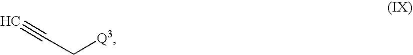
in which
Q³ represents a suitable leaving group, such as, for example, chlorine, bromine or iodine,
into compounds of the formula (X) 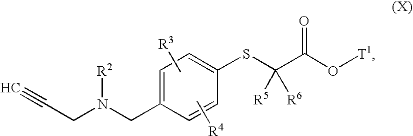
in which T¹, R², R³, R⁴, R⁵ and R⁶ are each as defined above,
these are then, in an inert solvent in the presence of N-chlorosuccinimide and a base, converted with a compound of the formula (XI) 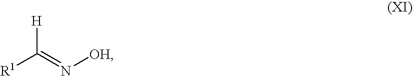
in which R¹ is as defined in claims 1 to 6,
via a 1,3-dipolar cycloaddition in compounds of the formula (I-H) 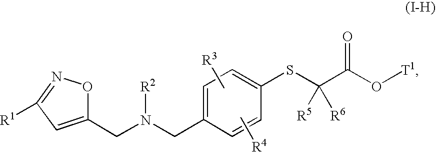
in which T¹, R¹, R², R³, R⁴, R⁵ and R⁶ are each as defined above,
then, by basic or acidic hydrolysis or, in the case that T¹ represents benzyl, also hydrogenolytically, converted into carboxylic acids of the formula (I-K) 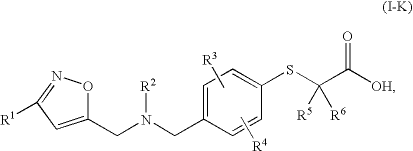
in which R¹, R², R³, R⁴, R⁵ and R⁶ are each as defined above,
and, if appropriate, finally converted by esterification or amidation methods known from the literature into the compounds of the formula (I-G).

10. Process for preparing a compound of the formula (I-L) 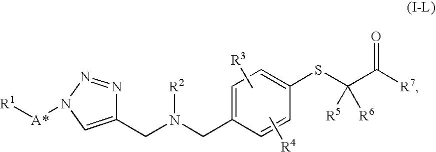
in which A* represents a CH₂ group or represents a bond and R¹, R², R³, R⁴, R⁵, R⁶ and R⁷ are each as defined in claims 1 to 6, characterized in that compounds of the formula (X) 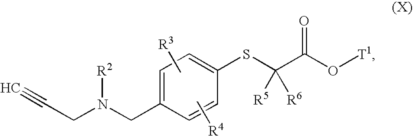
in which T¹, R², R³, R⁴, R⁵ and R⁶ are each as defined in claims 1 to 6
are converted, in an inert solvent in the presence of a copper(I) catalyst with an azide of the formula (XVI)
R¹-A*—N₃ (XVI),
in which R¹ is as defined in claims 1 to 6
and
A* represents a bond or represents a CH₂ group
via a 1,3-dipolar cycloaddition into compounds of the formula (I-M) 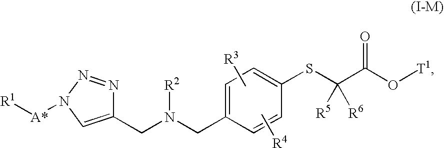
in which A*, T¹, R¹, R², R³, R⁴, R⁵ and R⁶ are each as defined above,
subsequently, by basic or acidic hydrolysis, converted into carboxylic acids of the formula (I-N) 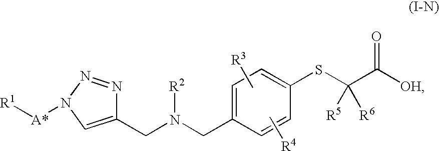
in which A*, R¹, R², R³, R⁴, R⁵ and R⁶ are each as defined above,
and, if appropriage, finally converted by esterification or amidation methods known from the literature into the compounds of the formula (I-L).

11. Compound as defined in any of claims 1 to 6 for the treatment and/or prophylaxis of diseases.

12. Use of a compound as defined in any of claims 1 to 6 for preparing a medicament for the treatment and/or prevention of dyslipidaemias and arteriosclerosis.

13. Medicament, comprising a compound as defined in any of claims 1 to 6 in combination with an inert non-toxic pharmaceutically suitable auxiliary.

14. Medicament, comprising a compound as defined in any of claims 1 to 6 in combination with a further active compound selected from the group consisting of CETP inhibitors, HMG-CoA reductase inhibitors, squalene synthesis inhibitors, ACAT inhibitors, cholesterol absorption inhibitors, MTP inhibitors, fibrates, niacin, lipase inhibitors, PPAR-γ- and/or PPAR-δ agonists, thyroid hormones and/or thyroid mimetics, polymeric bile acid adsorbers, bile acid reabsorption inhibitors, antioxidants, cannabinoid receptor 1 antagonists, insulin and insulin derivatives, antidiabetics, calcium antagonists, angiotensin AII antagonists, ACE inhibitors, beta-receptor blockers, alpha-receptor blockers, diuretics, platelet aggregation inhibitors and anticoagulants.

15. Medicament according to claim 13 or 14 for the treatment and/or prevention of dyslipidaemias and arteriosclerosis.

16. Method for the treatment and/or prevention of dyslipidaemias and arteriosclerosis in humans and animals by administering an effective amount of at least one compound as defined in any of claims 1 to 6 or of a medicament as defined in any of claims 13 to 15.
